(19) United States
(12) Patent Application Publication
(10) Pub. No.: US 2026/0226567 A1
(43) Pub. Date: Aug. 6, 2026

(54) METHOD OF DETECTING ADENO-ASSOCIATED VIRUS (AAV)

(71) Applicant: ASCEND GENE AND CELL THERAPIES LTD, Altrincham (GB)

(72) Inventors: Felicia Thoennissen, Planegg (DE); Sonya Schermann, Planegg (DE); Renée Kober, Planegg (DE)

(73) Assignee: ASCEND GENE AND CELL THERAPIES LTD, Altrincham (GB)

(21) Appl. No.: 19/117,044

(22) PCT Filed: Sep. 28, 2023
(86) PCT No.: PCT/GB2023/052514

§ 371 Date: Mar. 29, 2025

(30) Foreign Application Priority Data
Sep. 29, 2022 (GB) 2214328.3

(51) Int. Cl. C12Q 1/70; C12Q 1/6806; C12Q 1/6825; C12Q 1/686

(52) U.S. Cl. CPC C12Q 1/701; C12Q 1/6806; C12Q 1/6825; C12Q 1/686

(57) ABSTRACT

The present invention relates to methods for assaying viral nucleic acid impurities that might be present in adeno-associated virus (AAV) in a sample comprising recombinant AAV (rAAV) particles. More particularly, the present invention relates to methods for detecting rAAV particles which comprise both rep and cap sequences, as well as kits therefor and related pharmaceutical compositions.

### CROSS-REFERENCE

This application claim the benefit of International Patent Application Serial No. PCT/GB2023/052514, filed on Sep. 28, 2023, which claims priority to UK Patent Application Serial No. GB2214328.3, filed on Sep. 29, 2022, which are each incorporated herein by reference in its entirety.

### INCORPORATION BY REFERENCE OF SEQUENCE LISTING

The present application is being filed along with a Sequence Listing in electronic format. The Sequence Listing is provided as a file entitled ASCEND-P003_SEQLIST.xml, created on Mar. 26, 2025, which is 25.2 kilobytes in size. The information in the electronic format of the Sequence Listing is incorporated by reference in its entirety.

### FIELD OF THE INVENTION

The present invention relates to methods for assaying viral nucleic acid impurities that might be present in adeno-associated virus (AAV) in a sample comprising recombinant AAV (rAAV) particles. More particularly, the present invention relates to methods for detecting rAAV particles which comprise both rep and cap sequences, as well as kits therefor and related pharmaceutical compositions.

### BACKGROUND TO THE INVENTION

Recombinant adeno-associated virus (rAAV) vectors have considerable potential for gene therapy due to their promising safety profile and their ability to transduce many tissues in vivo. However, it remains difficult to obtain high quality rAAV at high yield, and production of adeno-associated virus (AAV) is still quite difficult and scale-up of production to an industrial scale has been accomplished only to a limited degree.

rAAV for gene therapy are generally produced by providing cells with genetic material (e.g. plasmids) containing the AAV Rep and Cap functions and an rAAV “vector genome” which comprises the “payload” of the rAAV flanked by inverted terminal repeat (ITR) sequences which ensure packaging of this heterologous nucleic acid within the produced viral particles. rAAV production additionally requires the presence of various helper functions, which may be provided by co-infection with a helper virus (e.g. an adenovirus or herpes simplex virus—1) or by providing the helper functions separately to the cells. The cells are cultured under conditions suitable for the AAV to propagate within the cells, and rAAV are harvested from the cells.

The packaging of the heterologous nucleic acid, e.g. recombinant DNA, into AAV vectors may inadvertently result in the packaging (encapsidation) of rep and cap nucleic acid sequences in an AAV particle. If the rep and cap nucleic acid sequences provide functional rep and cap genes, the resulting rAAV may be replication competent under conditions where the helper functions were also present. An AAV particle which comprises both rep and cap genes (such AAV particles are referred to as “pseudo-wild type AAV”, or “pseudo-wild type rcAAV”) may be able to replicate in a host cell without coinfection by another AAV particle if it also includes ITRs (in the presence of the necessary helper virus functions, but without any requirement for AAV functions provided in trans). The production of rAAV thus incurs the risk of generating replication-competent AAV (rcAAV).

For safety reasons, rAAV for use in gene therapy are typically required to be replication defective (or in other words, not to comprise functional AAV rep and cap genes). This is to reduce the risk that the rAAV might be able to undergo replication should a patient receiving AAV-based gene therapy be infected with a helper virus (e.g. an adenovirus).

Replication-competent AAV (rcAAV) may result from recombination events of the packaging plasmids. Split two plasmid systems (designated here as “split systems”) such as those described in WO2020/208379 and WO2022/079429 were designed to significantly reduce the risk of rcAAV generation by separating the sequences of the cap and the rep genes on two different plasmids (by contrast, conventionally used packaging systems harbour both genes on one plasmid (designated here as “non-split systems”)). FIG. 1 illustrates the production of rAAV by two different plasmid systems (a “non-split system” and a “split plasmid system”), and shows how unwanted side-products which could potentially provide rcAAV might potentially be produced from each system.

The current ‘standard’ test for detecting rcAAV in a sample comprising rAAV comprises transducing cells, such as HEK293 or HeLa cells, with the rAAV in the presence of a helper virus (e.g. adenovirus), and detecting the presence of a rep or cap nucleic acid molecule (also referred to as a rep sequence or a cap sequence, respectively) by qPCR after two or three rounds of viral amplification. This test is herein referred to as an “rcAAV assay”. More refined methods, in which the level of a rep or cap sequence is measured after each round of amplification have also been developed, as reported in WO2020/208379, whereby an increase in the detected level of the rep or cap sequence copy number per cell between successive amplification cycles indicates the presence of rAAV. However, these methods may not be sensitive enough to measure very low (<100 rcAAV in 1×IO¹⁰ AAV vector genomes) levels of rcAAV, and may not allow the user to visualise differences in the levels of rcAAV that might arise from using different rAAV production systems, for example, different plasmid systems for providing the various components required for rAAV production. This test may, therefore, potentially fail to detect low levels of rcAAV present in an rAAV sample, and may also not be suitable for measuring rcAAV levels where rAAV production systems are being optimised e.g. to minimise rcAAV. Furthermore, this test may incorrectly indicate the presence of rcAAV if a single cell is co-transduced separately with a first AAV comprising a functional rep gene, and a second AAV comprising a functional cap gene (as the second AAV may supply the cap function for the first AAV).

A number of additional methods for detecting rcAAV have been developed in order to attempt to overcome these shortcomings. For example, in WO2020/208379, qPCR and Southern blot assays were developed to quantify the levels of rep and cap nucleic acid molecules in an rAAV sample, and to visualise the rep and cap nucleic acid molecules that are present. However, these methods only report the relative levels of the rep and cap nucleic acid molecules present in the sample, without giving any indication of whether the rep and cap nucleic acid molecules are provided together (i.e. whether they are linked) in a single rAAV particle. Furthermore, these methods indicate the presence of highly heterogeneous rep and cap nucleic acid molecules. Additional PCR methods have also been developed to visualise rep and cap nucleic acid molecules present in an rAAV sample that might be linked, but quantifying the level of linked sequences is difficult, and these methods also depend on the respective species being present in specific orientations relative to one-another. The methods in WO2020/208379 are also performed following amplification of rcAAV in HEK293T cells to enrich samples for rcAAV.

There is therefore a need for a highly sensitive assay for detecting and accurately quantifying AAV particles in a rAAV sample which comprise both (linked) rep and cap nucleic acid molecules.

### SUMMARY OF THE INVENTION

The present invention provides methods for assaying for rAAV particles in a sample which comprise nucleotide sequences corresponding to at least a portion of a rep gene and at least a portion of a cap gene, as well as kits therefor and related pharmaceutical compositions. The assaying methods may comprise detecting and/or quantifying such rAAV particles. Detection of rAAV particles which comprise at least a portion of a rep gene and at least a portion of a cap gene may indicate the presence of rAAV particles in the sample which are pseudo-wild type rcAAV.

The methods described herein can therefore be used to determine the frequency of recombination events between rep gene sequences and cap gene sequences for all manufacturing platforms delivering rep and cap gene sequences on separate nucleic acid delivery entities (e.g. some insect cell/baculovirus platforms, or split plasmid systems as described in WO2020/208379 and WO2022/079429). Furthermore, the described methods provide an improved and more specific readout use in existing assays (such as an rcAAV assay described above) for detecting pseudo-wild type replication competent rAAV particles (i.e. a vector genome carrying a minimum of a functional rep gene sequence and a functional cap gene sequence in either orientation and which are replicating in transcomplementing cell lines infected with adenovirus). Presently, in the standard rcAAV assay final detection of amplified genomes is performed based on detection of rep or cap by qPCR. Therefore, any genomes that contain rep only or cap only flanked by ITRs. and which can replicate if the other functions are delivered in trans are detected as rcAAV. For example, any cell that is co-transduced by individual rep and cap containing particles in the presence of a helper virus can replicate (because all necessary genes for genome amplification and packaging into progeny virus are delivered into the same cell) and these can be detected by rep only or cap only qPCR, producing false positive signals.

The methods described herein can be used to support product development and/or process optimization by rapidly assessing the presence and/or quantity of rAAV particles comprising (linked) rep and cap gene sequences packed in the same rAAV particle and which result, for example, from modified processes or starting materials.

Thus, in a first aspect, the present invention provides a method of detecting adeno-associated virus (AAV) in a sample comprising recombinant adeno-associated virus (rAAV) particles which may be pseudo-wild type rAAV particles, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. detecting any compartments, within the plurality of compartments, that comprise, as indicated by the assays of step (c):
   i) a nucleotide sequence corresponding to the at least a portion of the rep gene but do not comprise a nucleotide sequence corresponding to the at least a portion of the cap gene;
   ii) a nucleotide sequence corresponding to the at least a portion of the cap gene but do not comprise the nucleotide sequence corresponding to the at least a portion of the rep gene; and
   iii) a nucleotide sequence corresponding to the at least a portion of the rep gene and a nucleotide sequence corresponding to the at least a portion of the cap gene;
      wherein the detection of compartments that comprise both a nucleotide sequence corresponding to the at least a portion of the rep gene and a nucleotide sequence corresponding to the at least a portion of the cap gene indicates that the sample comprises rAAV particles which may be pseudo-wild type rAAV particles.

In a second aspect, the invention provides a method of quantifying adeno-associated virus (AAV) in a sample comprising recombinant adeno-associated virus (rAAV) particles which may be pseudo-wild type rAAV particles, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. determining the number of compartments, within the plurality of compartments, that comprise, as indicated by the assays of step (c):
   i) a nucleotide sequence corresponding to the at least a portion of the rep gene but not a nucleotide sequence corresponding to the at least a portion of the cap gene;
   ii) a nucleotide sequence corresponding to the at least a portion of the cap gene but not a nucleotide sequence corresponding to the at least a portion of the rep gene; and
   iii) a nucleotide sequence corresponding to the at least a portion of the rep gene and a nucleotide sequence corresponding to the at least a portion of the cap gene;
      wherein the number of compartments that comprise both a nucleotide sequence corresponding to the at least a portion of the rep gene and a nucleotide sequence corresponding to the at least a portion of the cap gene indicates the quantity of rAAV in the sample which may be pseudo-wild type rAAV particles.

In a third aspect, the present invention provides a kit comprising:
(a) forward and reverse PCR primers capable of hybridising to an AAV rep gene; and
(b) forward and reverse PCR primers capable of hybridising to an AAV cap gene.

In a fourth aspect, the invention provides pharmaceutical composition comprising rAAV particles, wherein the proportion of AAV particles in the pharmaceutical composition which may be pseudo-wild type rAAV particles was determined by a method of the invention.

### DESCRIPTION OF THE FIGURES

FIG. 1: While AAV vectors are engineered to be replication defective, generation of replication-competent AAV (rcAAV) can still occur during vector manufacturing by means of recombination events within the producer cells. (A) In a conventional non-split plasmid system sequences encoding rep and cap are comprised on one plasmid (helper plasmid), with a gene of interest encoding a therapeutic protein comprised between a pair of ITR sequences on a second plasmid (vector plasmid). (B) Only one recombination event between the two plasmids is required to form a vector genome comprising rep and cap sequences, which may give rise to replication-competent AAV (rcAAV). (C) In a transsplit two-plasmid system, rep and cap sequences are comprised on separate plasmids. (D) A single recombination event could result in linked rep-cap species (E). These would not be flanked by ITR sequences, however, and therefore would not be packaged into rAAV particles and thus rAAV which are considered “replication-competent” would not be produced (as rcAAV require ITRs). (F) One recombination event results in a cap-only AAV, which is not replication competent. Similarly, rep could recombine in a rep-only AAV (not shown). (G) A second recombination event is required to generate an rAAV particle comprising both rep and cap nucleic acid sequences, which may be replication-competent AAV (rcAAV) (H). ITR=inverted terminal repeat. GOI=gene of interest.

FIG. 2 provides a schematic of the Rep78neg helper plasmid constructed as described in the Examples, with inset showing rep genes including p5, pl9 and p40 promoters. The crosses through “p40” indicate that these promoters have been rendered non-functional. Hatched area in the rep 52/40 gene indicates the presence of intron sequence which is spliced into rep 52 transcript but spliced out of rep 40 transcript. Ori=bacterial origin of replication. KanR=kanamycin resistance gene. Note the respective plasmid features are not shown to scale.

FIG. 3 provides a schematic of Rep78pos helper plasmid (non-optimised) constructed as described in the Examples, with inset showing rep genes including p5, pl9 and p40 promoters. The cross through “p40” indicates that this promoter has been rendered nonfunctional. Hatched area indicates the presence of intron sequence which is spliced into rep 52 and rep 78 transcripts but spliced out of rep 40 and rep 68 transcripts. The * indicates the presence of sequence CT AG (a cloning artefact) between the nucleotides which correspond to AAV2 positions 2329 and 4171. Ori=bacterial origin of replication. KanR=kanamycin resistance gene. Note the respective plasmid features are not shown to scale.

FIG. 4 provides a schematic of Rep78pos helper plasmid (optimised) constructed as described in the Examples, with inset showing rep genes including p5, pl9 and p40 promoters. The cross through “p40” indicates that this promoter has been rendered nonfunctional. Hatched area indicates the presence of intron sequence which is spliced into rep 52 and rep 78 transcripts but spliced out of rep 40 and rep 68 transcripts. Ori=bacterial origin of replication. KanR=kanamycin resistance gene. Note the respective plasmid features are not shown to scale.

FIG. 5 provides a schematic of vector plasmid, with inset showing cap gene and upstream promoter region including p5, pl9 and p40 promoters and functional cap genes encoding VP1, VP2 and VP3 Cap proteins, constructed as described in the Examples. The crosses through “ATG” and “GTG” indicate that these potential translation initiation codons have been deleted. Ori=bacterial origin of replication. KanR=kanamycin resistance gene. ITR=inverted terminal repeat. Note the respective plasmid features are not shown to scale.

FIG. 6 provides a schematic of the P-143 plasmid encoding complete Rep and Cap. The promoters (p5, pl9 and p40) are indicated The crosses through “p40” indicate that these promoters have been rendered non-functional. Note the respective plasmid features are not shown to scale.

FIG. 7 provides a schematic of how exemplary primer and probe combinations used herein may allow the detection of rep and cap nucleic acid sequences. Forward primer Q-008 and reverse primer Q-007 are used to amplify a sequence in the rep68 AAV gene. This amplification product is detected with the FAM-labelled probe QP-048. Similarly, forward primer Q-010 and reverse primer Q-009 are used to amplify a sequence in the cap AAV gene. This amplification product is detected with the HEX-labelled probe QP-049. (A & B) Linked rep and cap sequences can be detected, independent of the orientation of rep and cap on the molecule. (C) Unlinked rep and cap sequences can also be detected where one or both sequences are packaged in one rAAV particle. Unlinked sequences are less likely to both be packaged in a single rAAV particle than linked rep-cap species. rAAV particles comprising only rep nucleic acid sequence or only a cap nucleic acid sequence can be detected and ratios of rep-only or cap-only rAAV particles to linked rep and cap sequences can be calculated. ITR=inverted terminal repeat.

FIG. 8 provides the sequence of SEQ ID NOs: 1 and 11.

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a method comprising” particular steps should be interpreted to mean that the method includes those steps, but the method may comprise further steps. In some embodiments, “comprising” may be replaced by “consisting”.

The terms “protein” and “polypeptide” are used interchangeably herein, and are intended to refer to a polymeric chain of amino acids of any length.

The terms “nucleic acid molecule”, “nucleic acid sequence”, “polynucleotide” and “nucleotide sequence” are used interchangeably herein, and are intended to refer to a polymeric chain of nucleotides of any length e.g. deoxyribonucleotides, ribonucleotides, or analogs thereof. For example, the polynucleotide may comprise DNA (deoxyribonucleotides) or RNA (ribonucleotides). The polynucleotide may consist of DNA. The polynucleotide may be mRNA. Since the polynucleotide may comprise RNA or DNA, all references to T (thymine) nucleotides may be replaced with U (uracil).

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotides or amino acids at each position are then compared. When a position in the first sequence is occupied by the same amino acid or nucleotide as the corresponding position in the second sequence, then the amino acids or nucleotides are identical at that position. The percent identity between the two sequences is a function of the number of identical positions shared by the sequences (i.e., % identity=number of identical positions/total number of positions in the reference sequence×100).

Typically, the sequence comparison is carried out over the length of the reference sequence. For example, if the user wished to determine whether a given (“test”) sequence is 95% identical to SEQ ID NO: 1, SEQ ID NO: 1 would be the reference sequence. To assess whether a sequence is at least 95% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 95% of the positions are identical, the test sequence is at least 95% identical to SEQ ID NO: 1 (i.e., has at least 95% identity to SEQ ID NO: 1). If the sequence is shorter than SEQ ID NO: 1, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the EMBOSS Needle Pairwise Sequence Alignment tool.

Herein, the term “primer” is intended to refer to an oligonucleotide that hybridises to a nucleic acid molecule and can initiate elongation at its 3′ end. A “primer pair” (e.g. a primer pair for use in a PCR reaction) refers to an oligonucleotide set consisting of: (a) a forward primer that hybridises to a first location of a nucleic acid sequence; and (b) a reverse primer that hybridises to a second location of the nucleic acid sequence downstream of the first location. Therefore, the forward primer typically hybridises to one strand of a double stranded nucleotide sequence, and the reverse primer typically hybridises to the other strand of the double stranded nucleotide sequence.

A nucleic acid molecule “capable of hybridising” to a particular nucleic acid target comprises ribonucleotides or deoxyribonucleotides that are capable of participating in Watson-Crick or analogous base pair interactions with the target resulting in the formation of a duplex. Hybridisation may occur under suitable conditions, e.g. at a suitable temperature and/or in a buffer having a suitable pH and/or ionic strength.

Herein, the term “amplification product” is intended to refer to a nucleic acid fragment (i.e., an “amplicon”) formed as a product of natural or artificial amplification events or techniques. For example, an amplification product can be produced by PCR, such as ddPCR, ligase chain reaction, or gene duplication.

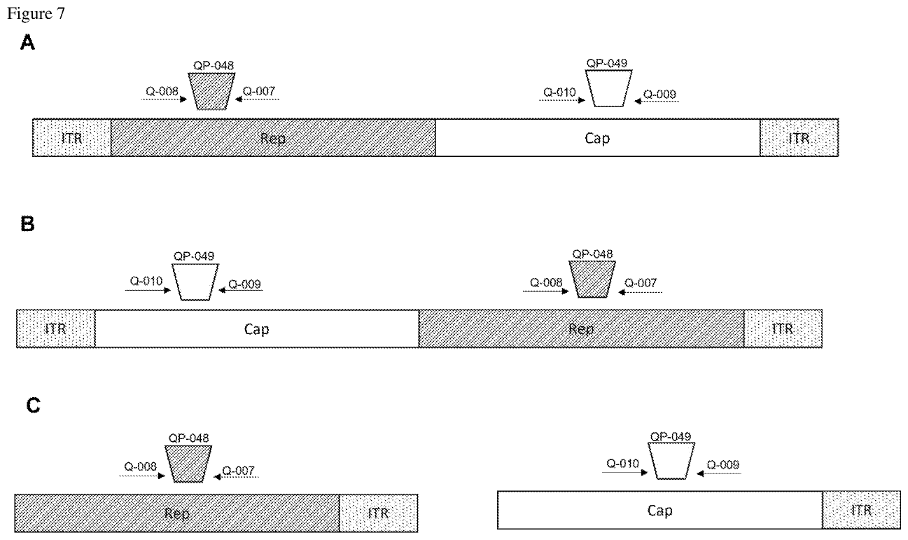

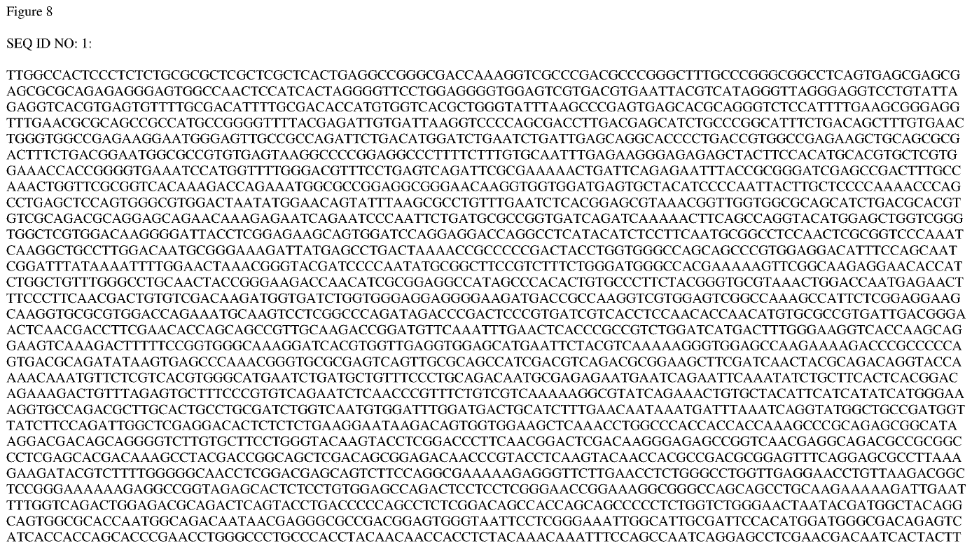

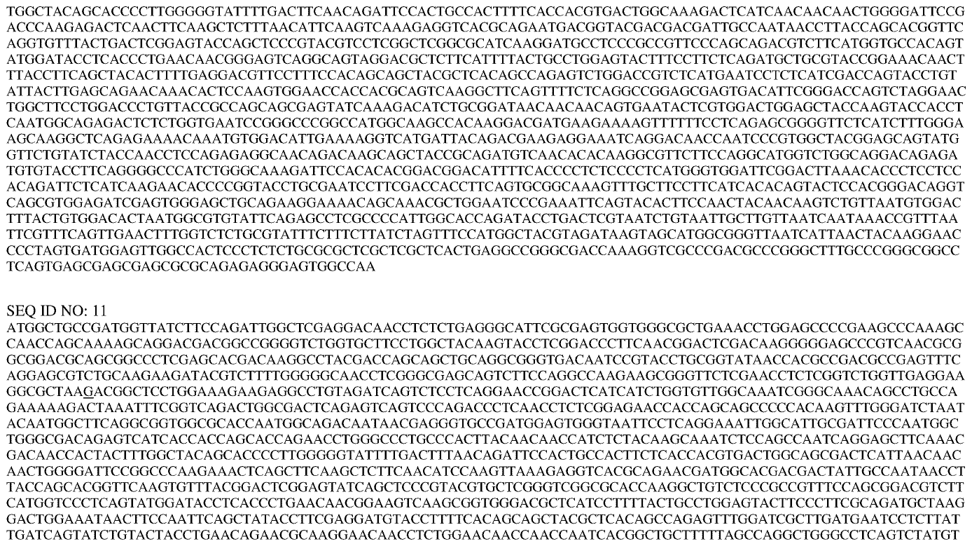

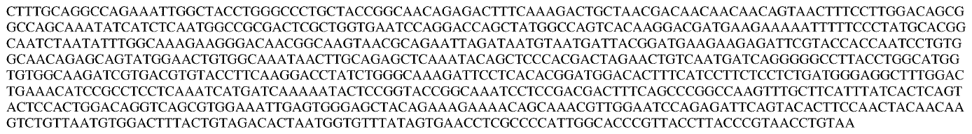

The term “at least a portion of a rep gene” is intended to refer to a nucleotide sequence that would align to a region of a rep gene that is being assayed for. Correspondingly, “o/ least a portion of a cap gene” is intended to refer to a nucleotide sequence that would align to a region of a cap gene that is being assayed for. Typically, the “at least a portion of a rep gene” and the “at least a portion of a cap gene” may be detected by amplification using suitable primers, for example, as shown in FIG. 7.

The terms “replication-competent AAV”, “recombinant AAV particle which is replication competent” and “rcAAV” are used interchangeably herein, and refer to an AAV particle which comprises a sequence flanked by inverted terminal repeats (ITRs) which is capable of replicating under conditions where the helper functions are also present.

The terms “pseudo-wild type AAV” and “pseudo-wild type rAAV” are used interchangeably herein, and refer to an AAV particle which comprises both Rep and Cap functions (i.e. which comprises a rep gene encoding a functional set of Rep proteins, and a cap gene encoding functional set of Cap proteins). A pseudo-wild type rAAV particle may provide rep and cap functions that can allow rAAV production if a cell is co-infected with an rcAAV particle in the presence of helper virus functions. Optionally, a pseudo-wild type rAAV may comprise a rep gene and a cap gene flanked by ITR sequences, i.e. the pseudo-wild type rAAV may be pseudo-wild type rcAAV. Pseudo-wild type rcAAV may (in the presence of helper virus functions) be able to replicate in a host cell without coinfection by another AAV particle, and produce progeny virus.

Analogously, the term “rAA V particle which may be pseudo-wild type” refers to an AAV particle which comprises both a nucleotide sequence corresponding to at least a portion of a rep gene and a nucleotide sequence corresponding to at least a portion of a cap gene i.e. which comprises linked rep and cap sequences). This term does not imply that the AAV particle is necessarily replication competent; for example, the AAV particle may comprise only a portion of a rep gene that is not sufficient to provide Rep functions, and/or only a portion of a cap gene that is not sufficient to provide Cap functions. Rather, the term “may be pseudo-wild type AA” implies that the AAV particle comprises nucleotide sequences that would provide Rep and Cap functions (i.e. that the AAV particle may potentially be a pseudo-wild type AAV particle), should the AAV particle comprise sufficient portions of the rep and cap genes to provide these functions.

Detecting rAAV particles which may be pseudo-wild type is a measure of rAAV particles in a sample which are pseudo-wild type (and indeed pseudo-wild type rcAAV), and may, for example, be useful to determine whether different production methods generate AAV particles with different levels of pseudo-wild type AAV. Furthermore, in the methods of the invention for detecting AAV in a sample which may be pseudo-wild type, the sample may comprise both rAAV which may be pseudo-wild type, and rAAV which are not pseudo-wild type, and typically only a low proportion of the rAAV in the sample (e.g. 1 in 10⁷ or 1 in 10⁸ rAAV may be pseudowild type).

Herein, the term “detecting” in the context of detecting rAAV in a sample which may be pseudo-wild type is intended to refer to determining the presence or absence, and optionally, the quantity of, rAAV particles in the sample which may be pseudo-wild type. The presence of compartments in which a nucleotide sequence corresponding to at least a portion of a rep gene and a nucleotide sequence corresponding to at least a portion of a cap gene are present indicates whether or not the sample contains rAAV particles comprising at least a portion of a rep gene and at least a portion of a cap gene, which may be linked or which may be co-packaged. Such particles may be pseudo-wild type.

The term “quantifying” in the context of quantifying AAV in a sample which may be pseudo-wild type is intended to refer to indicating the quantity of rAAV particles in the sample (or optionally, the proportion of rAAV particles in the sample) which may be pseudo-wild type.

The frequency of compartments in which a nucleotide sequence corresponding to at least a portion of a rep gene and a nucleotide sequence corresponding to at least a portion of a cap gene are present provides a measure of the quantity of rAAV particles in the sample (or optionally, the proportion of rAAV particles in the sample) comprising at least a portion of a rep gene and at least a portion of a cap gene, which may be linked or which may be co-packaged. Such particles may be pseudo-wild type.

Herein, the term “plasmid” is intended to refer to a nucleic acid molecule that can replicate independently of a cell chromosome. The term “plasmid” is intended to cover circular nucleic acid molecules and linear nucleic acid molecules. Furthermore, the term “plasmid” is intended to cover bacterial plasmids, but also cosmids, minicircles (Nehlsen, K., Broil S., Bode, J. (2006), Gene Ther. Mol. Biol., 10: 233-244; Kay, M. A., He, C.-Y, Chen, Z.-H. (2010), Nature Biotechnology, 28: 1287-1289) and ministrings (Nafissi N, Alqawlaq S, Lee E A, Foldvari M, Spagnuolo P A, Slavcev R A. (2014), Mol Ther Nucleic 15 Acids, 3:el65). Optionally, the plasmid is a circular nucleic acid molecule. Optionally, the plasmid is a nucleic acid molecule that is of bacterial origin. The plasmid may comprise at least one inverted terminal repeat (ITR). Thus, optionally, the one or more plasmids may comprise at least one ITR, but, more typically, two ITRs (generally with one either end of the expression cassette comprising the gene of interest, i.e. one at the 5′ end and one at the 3′ end). Optionally, the at least one ITR is an AAV ITR.

Optionally, the at least one ITR is an AAV-derived ITR. There may be intervening sequences between the expression cassette and one or more of the ITRs. The expression cassette may be incorporated into a viral particle located between two regular ITRs or located on either side of an ITR engineered with two D regions. Optionally, the ITR sequences are derived from AAV1, AAV2, AAV4 and/or AAV6.

The term “around” or “about” in relation to a reference numerical value and its grammatical equivalents as used herein can include the numerical value itself and a range of values plus or minus 10% from that numerical value. For example, the term “around” or “about” in relation to a reference numerical value can also include a range of values plus or minus 10%, 9%, 8%, 7%, 6%, 5%, 4%, 3%, 2%, or 1% from that value. For example, reference to a wavelength of “around” 520 nm may refer to a wavelength of 515-525 nm.

It is to be understood, although not always explicitly stated that in some embodiments, all numerical designations are preceded by the term “about”.

The term “between” and its grammatical equivalents in relation to a pair of reference numerical values and its grammatical equivalents as used herein can include the numerical values themselves and the range of values between the reference numerical values. For example, the term “between 1 and 3 substitutions” may refer to 1, 2 or 3 substitutions.

The singular forms “a”, “an”, and “the” include plural referents unless the content clearly dictates otherwise. Thus, for example, reference to “a nucleotide sequence corresponding to at least a portion of the rep gene” includes two or more instances or versions of nucleotides corresponding to at least portions of the rep gene.

All publications, patents and patent applications cited herein, whether supra or infra, are hereby incorporated by reference in their entirety.

Assaying for rAAV which Comprise Impurities which are Derived from AAV Rep and Cap Genes

The present invention provides methods related to the detection of nucleotide impurities in rAAV particles which are derived from AAV rep and cap genes.

In a first aspect, the present invention provides a method of detecting adeno-associated virus (AAV) in a sample comprising recombinant adeno-associated virus (rAAV) particles which may be pseudo-wild type rAAV particles, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. detecting any compartments, within the plurality of compartments, that comprise, as indicated by the assays of step (c):
   i) a nucleotide sequence corresponding to the at least a portion of the rep gene but do not comprise a nucleotide sequence corresponding to the at least a portion of the cap gene;
   ii) a nucleotide sequence corresponding to the at least a portion of the cap gene but do not comprise the nucleotide sequence corresponding to the at least a portion of the rep gene; and
   iii) a nucleotide sequence corresponding to the at least a portion of the rep gene and a nucleotide sequence corresponding to the at least a portion of the cap gene;
      wherein the detection of compartments that comprise both a nucleotide sequence corresponding to the at least a portion of the rep gene and a nucleotide sequence corresponding to the at least a portion of the cap gene indicates that the sample comprises rAAV particles which may be pseudo-wild type rAAV particles.

Optionally, step (d) comprises determining the number of compartments that comprise a nucleotide sequence corresponding to the at least a portion of the rep gene and a nucleotide sequence corresponding to the at least a portion of the cap gene, wherein the number of compartments that comprise both a nucleotide sequence corresponding to the at least a portion of the rep gene and a nucleotide sequence corresponding to the at least a portion of the cap gene indicates the quantity of rAAV in the sample which may be pseudo-wild type rAAV particles.

Thus, in another aspect, the present invention provides a method of quantifying adeno-associated virus (AAV) in a sample comprising recombinant adeno-associated virus (rAAV) particles which may be pseudo-wild type rAAV particles, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. determining the number of compartments, within the plurality of compartments, that comprise, as indicated by the assays of step (c):
   i) a nucleotide sequence corresponding to the at least a portion of the rep gene but not a nucleotide sequence corresponding to the at least a portion of the cap gene;
   ii) a nucleotide sequence corresponding to the at least a portion of the cap gene but not a nucleotide sequence corresponding to the at least a portion of the rep gene; and
   iii) a nucleotide sequence corresponding to the at least a portion of the rep gene and a nucleotide sequence corresponding to the at least a portion of the cap gene;
      wherein the number of compartments that comprise both a nucleotide sequence corresponding to the at least a portion of the rep gene and a nucleotide sequence corresponding to the at least a portion of the cap gene indicates the quantity of rAAV in the sample which may be pseudo-wild type rAAV particles.

As discussed above, in order for an AAV particle to be pseudo-wild type, the AAV particle comprises both Rep and Cap functions.

AAV comprises a rep gene region which encodes four Rep proteins: Rep78, Rep68, Rep52 and Rep40. The gene region is under the control of the p5 and pl9 promoters. When the p5 promoter is used, a gene that encodes Rep78 and Rep68 is transcribed. Rep78 and Rep68 are two alternative splice variants (Rep78 comprises an intron that is excised in Rep68). Similarly, when the pl9 promoter is used, a gene that encodes Rep52 and Rep40 is transcribed. Rep52 and Rep40 are alternative splice variants (Rep52 comprises an intron that is excised in Rep40). The portions of the AAV rep gene that encode the Rep52 and Rep40 proteins also encode, respectively, part of the Rep78 and Rep68 proteins. There are also portions of the rep gene region which encode portions of all four of the Rep proteins, i.e. there are portions common to all four rep genes. Thus, for example, a nucleic acid probe capable of hybridising to a Rep52 protein may also be capable of hybridising to a sequence encoding a Rep78 protein, and a nucleic acid probe capable of hybridising to a sequence encoding a Rep40 protein may also be capable of hybridising to a sequence encoding a Rep68 protein.

The four Rep proteins are known to be involved in replication and packaging of the viral genome, and are, therefore, useful for AAV replication. An AAV particle which comprises Rep function comprises a rep gene which encodes for a “functional” set of Rep proteins. A “functional” set of Rep proteins is one which is sufficient for AAV replication in the presence of the other functions required therefor i.e. a functional set of Cap proteins and the required helper functions). It is within the abilities of the skilled person to determine whether the products of a rep gene are functional, e.g. by determining whether the encoded Rep proteins support AAV production using an AAV production assay, for example as described in WO 2020/208379.

AAV comprises a cap gene region which encodes three Cap proteins: VP1, VP2 and VP3. All of VP1, VP2 and VP3 are encoded in native AAV by a single gene, the cap gene. The amino acid sequence of VP1 comprises the sequence of VP2 and VP3. The portion of VP1 which does not form part of VP2 and VP3 is referred to as VPlunique, or VP1U. The amino acid sequence of VP2 (and of VP1) comprises the sequence of VP3. The portion of VP2 which does not form part of VP3 is referred to as VP2unique, or VP2U. Thus, a nucleic acid probe capable of hybridising to a sequence encoding a VP3 protein may also be capable of hybridising to sequences encoding the VP1 and VP2 proteins which comprise the sequence of the VP3 protein. Similarly, a nucleic acid probe capable of hybridising to a sequence encoding the VP2U portion of a VP2 protein may also be capable of hybridising to a sequence encoding the VP1 protein which comprises the sequence of that VP2 protein. However, a probe capable of hybridising to a sequence encoding a VP1U portion of a VP1 protein may not be capable of hybridising to a sequence encoding a VP2 or a VP3 protein, and a probe capable of hybridising to a sequence encoding a VP2U portion of a VP2 protein may not be capable of hybridising to a sequence encoding a VP3 protein.

An AAV particle which comprises Cap function comprises a cap gene which encodes for a “functional” set of Cap proteins. A “functional” set of Cap proteins is one which is sufficient for encapsidation of AAV. It is within the abilities of the skilled person to determine whether the product of a cap gene is functional, e.g. by determining whether the encoded Cap protein(s) support AAV production using an AAV production assay, for example as described in WO 2020/208379.

Thus, according to the present invention, a pseudo-wild type rAAV particle comprises a rep gene encoding a functional set of Rep proteins, and a cap gene encoding a functional set of Cap proteins.

The methods of the invention simply require assays for indicating the presence of a nucleotide sequence corresponding to at least a portion of a rep gene and the presence of a nucleotide sequence corresponding to at least a portion of a cap gene within each of the plurality of compartments to be performed. Whilst the presence of a nucleotide sequence corresponding to at least a portion of a rep gene may indicate the presence of a rep gene encoding a functional set of Rep proteins, and the presence of a nucleotide sequence corresponding to at least a portion of a cap gene may indicate the presence of a cap gene encoding a functional set of Cap proteins, the methods of the invention do not require any determination to be made as to whether the at least a portion of the rep gene encodes a functional set of Rep proteins, or whether the at least a portion of the cap gene encodes a functional set of Cap proteins. Rather, the presence of a nucleotide sequence corresponding to at least a portion of a rep gene may potentially, but not necessarily, be indicative of the presence of a rep gene encoding a functional set of Rep proteins, and the presence of a nucleotide sequence corresponding to at least a portion of a cap gene may potentially, but not necessarily, be indicative of the presence of a cap gene encoding a functional set of Cap proteins. Thus, together, the presence of both a nucleotide sequence corresponding to at least a portion of a rep gene and the presence of a nucleotide sequence corresponding to at least a portion of a cap gene in a compartment may indicate that the compartment comprises an rAAV that comprises both a rep gene encoding a functional set of Rep proteins, and a cap gene encoding a functional set of Cap proteins, and thus which is a pseudo-wild type rAAV particle. By contrast, if a nucleotide sequence corresponding to at least a portion of a rep gene and if a nucleotide sequence corresponding to at least a portion of a cap gene are not both detected (i.e. if only one or the other nucleotide sequences are detected, or neither nucleotide sequence is detected), an AAV particle that is a pseudo-wild type rAAV particle is not present. Thus, detecting compartments that comprise both a nucleotide sequence corresponding to the at least a portion of the rep gene and a nucleotide sequence corresponding to the at least a portion of the cap gene indicates that the sample comprises rAAV particles which may be pseudo-wild type rAAV particles.

Pseudo-wild type rAAV particles which comprise rep and cap genes flanked by inverted terminal repeat (ITR) sequences may be considered to be replication competent (and in particular, as discussed elsewhere herein, may be able to replicate in a host cell in the presence of helper virus functions without co-infection by another AAV particle). In order for a pseudo-wild type rAAV particle to be replication competent, the rAAV particle comprises ITR sequences. As outlined in greater detail below, the present invention provides methods for detecting and quantifying AAV in a sample comprising recombinant adeno-associated virus (rAAV) particles which may be pseudo-wild type rcAAV particles, by further detecting the presence of at least a portion of an ITR.

The ITR sequences are comparatively short (typically around 145-155 bp) sequences found at the 5′ and 3′ ends of an AAV genome. Each ITR sequence comprises self-complementary portions capable of forming a hairpin structure. According to the present invention, a rcAAV particle comprises a 5′ and a 3′ ITR sequence.

Some embodiments of the present invention may further require assays for indicating the presence of a nucleotide sequence corresponding to at least a portion of an ITR and a nucleotide sequence corresponding to at least a portion of a rep gene within each of the plurality of compartments to be performed. Whilst the presence of a nucleotide sequence corresponding to at least a portion of an ITR may indicate the presence of the 5′ or 3′ ITR, the methods of the invention do not require any determination to be made as to whether the complete ITR is present. Rather, the presence of a nucleotide sequence corresponding to at least a portion of an ITR may potentially, but not necessarily, be indicative of the presence of the complete ITR. Thus, the presence of a nucleotide sequence corresponding to at least a portion of an ITR in a compartment may indicate that the compartment comprises an rAAV that comprises 5′ and 3′ ITR sequences, and thus if it also comprises Rep and Cap functions is replication competent. By contrast, if a nucleotide sequence corresponding to at least a portion of an ITR is not detected in combination with a nucleotide sequence corresponding to at least a portion of a rep gene, a pseudo-wild type rcAAV particle is not present. Thus, detecting compartments that comprise both a nucleotide sequence corresponding to the at least a portion of the rep gene and a nucleotide sequence corresponding to the at least a portion of the cap gene indicates that the sample comprises rAAV particles which may be pseudo-wild type AAV particles, and detecting both a nucleotide sequence corresponding to the at least a portion of the rep gene and a nucleotide sequence corresponding to the at least a portion of an ITR indicates that the sample comprises rAAV particles which may be pseudo-wild type rcAAV particles.

The methods of the invention comprise detecting compartments which comprise a nucleotide sequence corresponding to at least a portion of a rep gene and a nucleotide sequence corresponding to at least a portion of a cap gene. Such compartment may be considered to represent compartments which comprise an rAAV that may be pseudo-wild type rAAV particles.

The nucleotide sequence corresponding to at least a portion of a rep gene and the nucleotide sequence corresponding to at least a portion of a cap gene may be encapsidated in a single rAAV particle. The present invention may, therefore, be considered to provide methods for detecting a nucleotide sequence corresponding to at least a portion of a rep gene and a nucleotide sequence corresponding to at least a portion of a cap gene which may be encapsidated in the same rAAV particle. Put another way, the present invention provides methods for the detection of rAAV particles which may comprise a nucleotide sequence corresponding to at least a portion of a rep gene and a nucleotide sequence corresponding to at least a portion of a cap gene (which may be linked or which may be copackaged). As discussed above, such rAAV particles may be pseudo-wild type rAAV particles, and/or may be replication competent.

In one aspect, the present invention therefore provides a method of detecting adeno-associated virus (AAV) in a sample comprising recombinant adeno-associated virus (rAAV) particles which may comprise a nucleotide sequence corresponding to at least a portion of a rep gene and a nucleotide sequence corresponding to at least a portion of a cap gene, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. detecting any compartments, within the plurality of compartments, that comprise, as indicated by the assays of step (c):
   i) a nucleotide sequence corresponding to the at least a portion of the rep gene but do not comprise a nucleotide sequence corresponding to the at least a portion of the cap gene;
   ii) a nucleotide sequence corresponding to the at least a portion of the cap gene but do not comprise the nucleotide sequence corresponding to the at least a portion of the rep gene; and
   iii) a nucleotide sequence corresponding to the at least a portion of the rep gene and a nucleotide sequence corresponding to the at least a portion of the cap gene;
      wherein the detection of compartments that comprise both a nucleotide sequence corresponding to the at least a portion of the rep gene and a nucleotide sequence corresponding to the at least a portion of the cap gene indicates that the sample comprises rAAV particles that may comprise a nucleotide sequence corresponding to at least a portion of a rep gene and a nucleotide sequence corresponding to at least a portion of a cap gene.

Optionally, step (d) comprises determining the number of compartments that comprise a nucleotide sequence corresponding to the at least a portion of the rep gene and a nucleotide sequence corresponding to the at least a portion of the cap gene, wherein the number of compartments that comprise both a nucleotide sequence corresponding to the at least a portion of the rep gene and a nucleotide sequence corresponding to the at least a portion of the cap gene indicates the quantity of rAAV in the sample comprising the rAAV particles which comprise a nucleotide sequence corresponding to at least a portion of a rep gene and a nucleotide sequence corresponding to at least a portion of a cap gene.

Thus, in another aspect the present invention provides a method of quantifying adeno-associated virus (AAV) in a sample comprising recombinant adeno-associated virus (rAAV) particles which may comprise a nucleotide sequence corresponding to at least a portion of a rep gene and a nucleotide sequence corresponding to at least a portion of a cap gene, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. determining the number of compartments, within the plurality of compartments, that comprise, as indicated by the assays of step (c):
   i) a nucleotide sequence corresponding to the at least a portion of the rep gene but not a nucleotide sequence corresponding to the at least a portion of the cap gene;
   ii) a nucleotide sequence corresponding to the at least a portion of the cap gene but not a nucleotide sequence corresponding to the at least a portion of the rep gene; and
   iii) a nucleotide sequence corresponding to the at least a portion of the rep gene and a nucleotide sequence corresponding to the at least a portion of the cap gene;
      wherein the number of compartments that comprise both a nucleotide sequence corresponding to the at least a portion of the rep gene and a nucleotide sequence corresponding to the at least a portion of the cap gene indicates the quantity of AAV in the sample comprising the rAAV particles which may comprise a nucleotide sequence corresponding to at least a portion of a rep gene and a nucleotide sequence corresponding to at least a portion of a cap gene.

Detecting both a nucleotide sequence corresponding to at least a portion of a rep gene and a nucleotide sequence corresponding to at least a portion of a cap gene within a compartment may indicate that the rep and cap nucleotide sequences are encapsidated within a single rAAV particle, particularly where partitioning step (b) is carried out in such a way as to minimise the number of compartments comprising two or more AAV particles which comprise at least one of a portion of the rep gene or the cap gene. Detecting both a nucleotide sequence corresponding to at least a portion of a rep gene and a nucleotide sequence corresponding to at least a portion of a cap gene within a compartment may indicate that the compartment (and thus the sample) comprises an rAAV particle which comprises both the rep and cap nucleotide sequences (i.e. that both the rep and cap nucleotide sequences are encapsidated within a single rAAV particle).

Certainly, detecting compartments which comprise a nucleotide sequence corresponding to at least a portion of a rep gene and a nucleotide sequence corresponding to at least a portion of a cap gene may still be indicative of the presence (or in quantitative methods, the quantity) of rAAV particles in the sample which comprise a nucleotide sequence corresponding to at least a portion of a rep gene and a nucleotide sequence corresponding to at least a portion of a cap gene, and thus have utility as methods for assessing the quality of a sample comprising rAAV.

Nucleotide sequences corresponding to at least a portion of a rep gene and at least a portion of a cap gene which are present within a compartment may be linked, or may be unlinked. “Linked” nucleotide sequences are nucleotide sequences which are covalently attached, i.e. which are provided as a single nucleic acid molecule. By way of example, the AAV genome (a single-stranded DNA genome) comprises both a rep gene and a cap gene, which genes are linked. Linked nucleotide sequences may be the result of a recombination event during the production of the rAAV. By contrast, “unlinked” nucleotide sequences are nucleotide sequences which are provided as separate nucleic acid molecules. Unlinked nucleotide sequences are likely to result from the rare co-packing of separate nucleotide sequences into a single rAAV particle or the co-partitioning of an rAAV particle comprising a nucleotide sequence corresponding to at least a portion of a rep gene and an rAAV particle comprising a nucleotide sequence corresponding to at least a portion of a cap gene within a single compartment.

The present invention may therefore be considered to provide methods for detecting nucleotide sequences corresponding to at least a portion of a rep gene and at least a portion of a cap gene which may be linked (i.e. linked rep-cap species) in a sample comprising rAAV particles. As discussed above, such linked sequences may be encapsidated in a single rAAV particle, and such rAAV particles may be pseudo-wild type rAAV particles and/or may be replication competent.

The present invention therefore provides a method for detecting AAV particles comprising nucleotide sequences corresponding to at least a portion of a rep gene and at least a portion of a cap gene which may be linked in a sample comprising rAAV particles, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. detecting any compartments, within the plurality of compartments, that comprise, as indicated by the assays of step (c):
   i) a nucleotide sequence corresponding to the at least a portion of the rep gene but do not comprise a nucleotide sequence corresponding to the at least a portion of the cap gene;
   ii) a nucleotide sequence corresponding to the at least a portion of the cap gene but do not comprise the nucleotide sequence corresponding to the at least a portion of the rep gene; and
   iii) a nucleotide sequence corresponding to the at least a portion of the rep gene and a nucleotide sequence corresponding to the at least a portion of the cap gene;
      wherein the detection of compartments that comprise both a nucleotide sequence corresponding to the at least a portion of the rep gene and a nucleotide sequence corresponding to the at least a portion of the cap gene indicates that the sample comprises AAV particles comprising nucleotide sequences corresponding to at least a portion of a rep gene and at least a portion of a cap gene which may be linked.

Optionally, step (d) comprises determining the number of compartments that comprise a nucleotide sequence corresponding to the at least a portion of the rep gene and a nucleotide sequence corresponding to the at least a portion of the cap gene, wherein the number of compartments that comprise both a nucleotide sequence corresponding to the at least a portion of the rep gene and a nucleotide sequence corresponding to the at least a portion of the cap gene indicates the quantity of nucleotide sequences corresponding to at least a portion of a rep gene and at least a portion of a cap gene which may be linked.

The present invention thus provides a method for quantifying AAV particles comprising linked rep-cap species in a sample comprising rAAV particles, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. determining the number of compartments, within the plurality of compartments, that comprise, as indicated by the assays of step (c):
   i) a nucleotide sequence corresponding to the at least a portion of the rep gene but not a nucleotide sequence corresponding to the at least a portion of the cap gene;
   ii) a nucleotide sequence corresponding to the at least a portion of the cap gene but not a nucleotide sequence corresponding to the at least a portion of the rep gene; and
   iii) a nucleotide sequence corresponding to the at least a portion of the rep gene and a nucleotide sequence corresponding to the at least a portion of the cap gene;
      wherein the number of compartments that comprise both a nucleotide sequence corresponding to the at least a portion of the rep gene and a nucleotide sequence corresponding to the at least a portion of the cap gene indicates the quantity of AAV particles comprising nucleotide sequences corresponding to at least a portion of a rep gene and at least a portion of a cap gene which may be linked.

Detecting both a nucleotide sequence corresponding to at least a portion of a rep gene and a nucleotide sequence corresponding to at least a portion of a cap gene within a compartment may indicate that the rep and cap sequences are linked. Detecting both a nucleotide sequence corresponding to at least a portion of a rep gene and a nucleotide sequence corresponding to at least a portion of a cap gene within a compartment may indicate that the compartment (and thus the sample) comprises linked rep and cap sequences. However, the nucleotide sequences corresponding to the at least a portion of the rep gene and the at least a portion of the cap gene may potentially be detected within the compartment if the nucleotide sequence corresponding to the at least a portion of the rep gene and the nucleotide sequence corresponding to the at least a portion of the cap are unlinked. Nevertheless, detecting compartments which comprise a nucleotide sequence corresponding to at least a portion of a rep gene and a nucleotide sequence corresponding to at least a portion of a cap gene may still be indicative of the presence (or in quantitative methods, the quantity) of linked rep and cap sequences in the sample, and thus have utility as methods for assessing the quality of a sample comprising rAAV.

According to certain alternative embodiments of the invention, step (d) of the methods for detecting AAV in a sample which may be pseudo-wild type AAV particles, AAV particles which may comprise a nucleotide sequence corresponding to at least a portion of a rep gene and a nucleotide sequence corresponding to at least a portion of a cap gene or AAV particles comprising nucleotide sequences corresponding to at least a portion of a rep gene and at least a portion of a cap gene which may be linked, may comprise detecting only the compartments, within the plurality of compartments, that comprise as indicated by the assays of step (c) a nucleotide sequence corresponding to the at least a portion of the rep gene and a nucleotide sequence corresponding to the at least a portion of the cap gene. In other words, optionally the present invention provides further methods for detecting AAV in a sample which may be pseudo-wild type AAV particles, AAV particles which may comprise a nucleotide sequence corresponding to at least a portion of a rep gene and a nucleotide sequence corresponding to at least a portion of a cap gene or AAV particles comprising nucleotide sequences corresponding to at least a portion of a rep gene and at least a portion of a cap gene which may be linked as described above, with the exception that step (d) does not comprise detecting compartments that comprise a nucleotide sequence corresponding to the at least a portion of the rep gene but do not comprise a nucleotide sequence corresponding to the at least a portion of the cap gene or compartments that comprise a nucleotide sequence corresponding to the at least a portion of the cap gene but do not comprise the nucleotide sequence corresponding to the at least a portion of the rep gene.

The Distribution of rAAV Particles within the Plurality of Compartments

The methods of the present invention comprise partitioning a sample into a plurality of compartments and detecting compartments which comprise i) a nucleotide sequence corresponding to at a least a portion of the rep gene but do not comprise a nucleotide sequence corresponding to at least a portion of the cap gene; ii) a nucleotide sequence corresponding to at least a portion of the cap gene but do not comprise the nucleotide sequence corresponding to at least a portion of the rep gene; and iii) a nucleotide sequence corresponding to at least a portion of the rep gene and a nucleotide sequence corresponding to at least a portion of the cap gene.

Detecting a nucleotide sequence corresponding to at least a portion of the rep gene and a nucleotide sequence corresponding to at least a portion of the cap gene can indicate the presence of rAAV particles which may be pseudo-wild type, which comprise both a rep and a cap sequence and/or which comprise linked rep and cap sequences, or simply linked rep and cap sequences.

If the compartments are of uniform volume, the number of rAAV particles which may be pseudo-wild type may be calculated by determining λ (the average number of rAAV particles which comprise a nucleotide sequence corresponding to at least a portion of a rep gene and a nucleotide sequence corresponding to at least a portion of a cap gene from the fraction of empty compartments and single and double positive end-point compartments using Poisson statistics, according to the equations set out in the supplementary information describing the mathematical relationships underlying linkage and droplet populations in Regan et al. 2015. PLoS One 10(3): e0l18270, the contents of which are hereby incorporated by reference in its entirety). From λ, together with the volume of each compartment and the total number of compartments analysed, an estimate of the absolute concentration of rAAV particles which may be pseudo-wild type can be obtained.

The distribution of rAAV particles (and nucleotide sequences corresponding to the at least a portion of the rep gene and to the at least a portion of the cap gene) within the sample is random and follows a Poisson distribution. Thus, some of the compartments may comprise more than one rAAV particle which comprises a nucleotide sequence corresponding to the at least a portion of the rep gene and/or a nucleotide sequence corresponding to the at least a portion of the cap gene. This may distort the results of the methods, and provide false-positive signals for rAAV particles which may be replication competent, which comprise both a rep and a cap sequence and/or which comprise linked rep and cap sequences, or linked rep and cap sequences. The methods of the invention may therefore be performed in such a way to minimise the probability that a compartment comprises more than one rep and/or cap sequence, for example, by diluting the rAAV sample.

Optionally, step (d) comprises detecting any compartments or determining the number of compartments, within the plurality of compartments, that do not comprise, as indicated by the assays of step (c), either a nucleotide sequence corresponding to a portion of the rep gene or a nucleotide sequence corresponding to a portion of the cap gene. Step (d) may, therefore, comprise detecting compartments or determining the number of compartments which comprise one of four possible combinations of nucleotide sequences which correspond to at least a portion of a rep gene and nucleotide sequences which correspond to at least a portion of a cap gene: compartments that do not comprise either a nucleotide sequence corresponding to at least a portion of a rep gene or a nucleotide sequence corresponding to at least a portion of a cap gene (rep⁻/cap⁻); compartments that comprise a nucleotide sequence corresponding to at least a portion of a rep gene but not a nucleotide sequence corresponding to at least a portion of a cap gene (rep⁺/cap⁻); compartments that comprise a nucleotide sequence corresponding to at least a portion of a cap gene but not a nucleotide sequence corresponding to at least a portion of a rep gene (rep⁻/cap⁺); and compartments that comprise a nucleotide sequence corresponding to at least a portion of a rep gene and a nucleotide sequence corresponding to at least a portion of a cap gene (rep⁺/cap⁺).

The skilled person may carry out the methods of the invention in such a manner so as to reduce the likelihood that a nucleotide sequence corresponding to at least a portion of a rep gene and a nucleotide sequence corresponding to at least a portion of a cap gene might co-localise in a compartment by chance, as opposed to being e.g. linked to one another or encapsidated in a single rAAV particle.

Optionally, fewer compartments comprise at least one of a nucleotide sequence corresponding to the at least a portion of the rep gene and a nucleotide sequence corresponding to the at least a portion of the cap gene (i.e. compartments which are rep⁺/cap⁻, rep⁻/cap⁺ or rep⁺/cap⁺) than the total number of compartments in the plurality of compartments i.e. optionally, not all of the compartments comprise at least one of a nucleotide sequence corresponding to the at least a portion of the rep gene and a nucleotide sequence corresponding to the at least a portion of the cap gene, and at least some of the compartments are rep-/cap-). Optionally, fewer than half of the compartments comprise at least one of a nucleotide sequence corresponding to the at least a portion of the rep gene and a nucleotide sequence corresponding to the at least a portion of the cap gene. Optionally, the ratio of the number of compartments comprising at least one of a nucleotide sequence corresponding to the at least a portion of the rep gene and a nucleotide sequence corresponding to the at least a portion of the cap gene to the total number of compartments in the plurality of compartments detected is 1:4 or less, 1:5 or less, 1:6 or less, 1:7 or less, 1:8 or less, 1:9 or less, 1:10 or less, 1:15 or less, 1:20 or less, or 1:25 or less.

As mentioned above, the distribution of rAAV particles within the plurality of compartments is random and the number of rAAV particles which are partitioned into each of the plurality of compartments follows a Poisson distribution. The skilled person may, therefore, be able to determine the modal number of rAAV particles which comprise a nucleotide sequence corresponding to the at least a portion of the rep gene and/or a nucleotide sequence corresponding to the at least a portion of the cap gene in each of the plurality of compartments (i.e. the most common number of rAAV particles which comprise a nucleotide sequence corresponding to the at least a portion of the rep gene and/or a nucleotide sequence corresponding to the at least a portion of the cap gene in each of the plurality of compartments). Optionally, the modal number of rAAV particles which comprise a nucleotide sequence corresponding to the at least a portion of the rep gene and/or a nucleotide sequence corresponding to the at least a portion of the cap gene in each of the plurality of compartments is 0. Optionally, at least 75%, at least 80%, at least 85%, at least 90% or at least 95% of the plurality of compartments does not comprise an AAV particle comprising a nucleotide sequence corresponding to the at least a portion of the rep gene and/or a nucleotide sequence corresponding to the at least a portion of the cap gene.

Optionally, the modal non-zero number of rAAV particles comprising a nucleotide sequence corresponding to the at least a portion of the rep gene and/or a nucleotide sequence corresponding to the at least a portion of the cap gene in each of the plurality of compartments is 1. In this context, the term “modal non-zero number” refers to the most common number of rAAV particles comprising a nucleotide sequence corresponding to the at least a portion of the rep gene and/or a nucleotide sequence corresponding to the at least a portion of the cap gene in each of the plurality of compartments that is not zero. By way of an illustrative example, if the number of rAAV particles comprising a nucleotide sequence corresponding to the at least a portion of the rep gene and/or a nucleotide sequence corresponding to the at least a portion of the cap gene in each of 10 compartments is 0, 1, 0, 0, 0, 2, 1, 0, 1, 0, the modal non-zero number would be 1.

The skilled person may optionally carry out the methods of the invention such that a low proportion of the plurality of compartments comprises two or more rAAV particles comprising a nucleotide sequence corresponding to the at least a portion of the rep gene and/or a nucleotide sequence corresponding to the at least a portion of the cap gene. Optionally, less than 5%, less than 2.5%, less than 2%, less than 1.5%, less than 1%, less than 0.5%, less than 0.4%, less than 0.3%, less than 0.2% or less than 0.1% of the plurality of compartments comprises two or more rAAV particles comprising a nucleotide sequence corresponding to the at least a portion of the rep gene and/or a nucleotide sequence corresponding to the at least a portion of the cap gene.

In the context of the present application, the proportion of compartments which comprise two or more rAAV particles comprising a nucleotide sequence corresponding to the at least a portion of the rep gene and/or a nucleotide sequence corresponding to the at least a portion of the cap gene can be optimised, for example, as mentioned above. However, the skilled person will understand that typically in a sample comprising rAAV, only a minority of the rAAV particles may comprise a nucleotide sequence corresponding to the at least a portion of the rep gene and/or a nucleotide sequence corresponding to the at least a portion of the cap gene (or in other words, the majority of the rAAV particles do not comprise a nucleotide sequence corresponding to the at least a portion of the rep gene and/or a nucleotide sequence corresponding to the at least a portion of the cap gene). Thus, although the methods may be optimised such that the number or proportion of compartments which comprise two or more rAAV particles which comprise a nucleotide sequence corresponding to the at least a portion of the rep gene and/or a nucleotide sequence corresponding to the at least a portion of the cap gene is low, this does restrict how many rAAV particles which do not comprise a nucleotide sequence corresponding to the at least a portion of the rep gene or a nucleotide sequence corresponding to the at least a portion of the cap gene may be present within the compartments.

The desired level of rAAV particles comprising a nucleotide sequence corresponding to the at least a portion of the rep gene and/or a nucleotide sequence corresponding to the at least a portion of the cap gene in each of the components may be achieved in a number of ways. Optionally, the compartments may be of a suitable volume to achieve the desired level of the rAAV particles. Optionally, the concentration of rAAV particles in the sample may be adjusted prior to partitioning the sample into a plurality of compartments.

Optionally, the sample is diluted before a step of partitioning takes place. Thus, optionally, the sample is diluted prior to step (b). Thus, a diluted sample may be obtained. Optionally, the diluted sample is partitioned into a plurality of compartments. Optionally, steps (b)-(d) are performed on the diluted sample. Optionally, the sample may be diluted by about 1.5× (i.e. 1.5 times), 2×, 5×, lO×, 25×, 50×, lOO×, 500×, lOOO×, 5000×, 10,000×, 50,000×, 100,000×, 500,000× or 1,000,000×.

Optionally, the sample is diluted by two or more dilution factors prior to step (b). Optionally at least a part of a sample is diluted by a first dilution factor, and at least a part of the sample is diluted by a second, different, dilution factor. Optionally, the sample is diluted by a serial dilution (a serial dilution may be particularly useful where higher dilution factors are used)—a serial dilution may be performed by diluting at least a part of the sample to provide a diluted sample, and further diluting at least a part of the diluted sample. Diluting the sample by two or more different dilution factors provides multiple (two or more) diluted samples having different dilutions. Steps (b)-(d) may then be performed on each of the multiple diluted samples.

Optionally, if, following performing steps (b)-(d) on the diluted sample or each of the multiple diluted samples, the ratio of the number of compartments comprising a nucleotide sequence corresponding to the at least a portion of the rep gene and/or a nucleotide sequence corresponding to the at least a portion of the cap gene to the total number of compartments in the plurality of compartments detected is greater than 1:4, greater than 1:5, greater than 1:6, greater than 1:7, greater than 1:8, greater than 1:9, greater than 1:10, greater than 1:15, greater than 1:20 or greater than 1:25 the user identifies that the dilution factor is too low and the sample is diluted by a further greater dilution factor and steps (b)-(d) are performed on the further diluted sample, optionally wherein the step of diluting the sample by a further dilution factor is repeated one or more times if, following performing steps (b)-(d) on a further diluted sample, the ratio of the number of compartments comprising a nucleotide sequence corresponding to the at least a portion of the rep gene and/or a nucleotide sequence corresponding to the at least a portion of the cap gene to the total number of compartments in the plurality of compartments detected is greater than 1:4, greater than 1:5, greater than 1:6, greater than 1:7, greater than 1:8, greater than 1:9, greater than 1:10, greater than 1:15, greater than 1:20, or greater than 1:25.

Optionally, not all of the compartments generated in step (b) are detected in step (d). Optionally, the compartments detected in the methods of the invention represent a sample of the compartments generated in step (b). Typically, a suitable number of compartments are detected in the methods of the invention to allow the detection of rare events, for example, the co-localisation of a rep gene and a cap gene in a single compartment. Optionally, the number of compartments detected in step (d) comprises at least 5000, at least 6000, at least 7000, at least 8000, at least 9000, at least 10,000, at least 12,000, at least 14,000, at least 16,000, at least 18,000, or at least 20,000 compartments. Optionally, the number of compartments detected in step (d) comprises fewer than 100,000, fewer than 75,000, fewer than 50,000, fewer than 40,000, fewer than 30,000 or fewer than 25,000 compartments. Optionally, the number of compartments detected in step (d) comprises 10,000-30,000 compartments or 15,000-25,000 compartments. Optionally, the number of compartments detected in step (d) comprises around 20,000 compartments. Optionally, the number of compartments detected in step (d) comprises 20,000 compartments.

### Assays for Indicating the Presence of Nucleotide Sequences

Any assay for indicating the presence of a nucleotide sequence in a sample may be employed in the methods of the invention to indicate the presence of a nucleotide sequence corresponding to at least a portion of a rep gene and the presence of a nucleotide sequence corresponding to at least a portion of a cap gene within each of the plurality of compartments.

Optionally, the assay comprises a step of amplifying a nucleotide sequence corresponding to at least a portion of a rep gene and/or a nucleotide sequence corresponding to at least a portion of a cap gene. The term “amplify” or “amplifying” is used to refer to a step of increasing the number of copies of a nucleotide sequence as a means to improve the sensitivity of a detection assay. Any amplification means known in the art may be used in the methods of the invention, for example, polymerase chain reaction (PCR), ligase chain reaction (LCR), rolling circle amplification (RCA), loop-mediated isothermal amplification (LAMP) or multiple displacement amplification (MDA). Optionally, the assay comprises the production of double-stranded DNA.

The methods of the invention may comprise performing assays for indicating the presence of any suitable portion(s) of a rep gene and/or a cap gene.

Optionally, the assay for indicating the presence of a nucleotide sequence corresponding to at least a portion of the rep gene comprises detecting at least a portion of a sequence encoding a Rep78 and/or a Rep68 protein or the complement thereof.

Optionally, the assay for indicating the presence of a nucleotide sequence corresponding to at least a portion of the rep gene comprises detecting at least a portion of a sequence corresponding to nucleotides 321-2252 of the AAV2 genome set forth in SEQ ID NO: 1 or the complement thereof. Optionally, the assay for indicating the presence of a nucleotide sequence corresponding to at least a portion of the rep gene comprises detecting at least a portion of a sequence at least 90%, at least 95%, at least 98%, at least 99% or 100% identical to nucleotides 321-2252 of SEQ ID NO: 1, or the complement thereof.

Optionally, the assay for indicating the presence of a nucleotide sequence corresponding to at least a portion of the rep gene does not comprise detecting sequences that encode a Rep52 and/or a Rep40 protein or the complement thereof. Thus, optionally, the assay for indicating the presence of a nucleotide sequence corresponding to at least a portion of the rep gene comprises detecting at least a portion of the Rep78/Rep68 unique portion of the AAV rep gene or the complement thereof. Optionally, the assay for indicating the presence of a nucleotide sequence corresponding to at least a portion of the rep gene comprises detecting at least a portion of a sequence corresponding to positions 321-992 of the AAV2 genome set forth in SEQ ID NO: 1, or the complement thereof. Optionally, the assay for indicating the presence of a nucleotide sequence corresponding to at least a portion of the rep gene comprises detecting at least a portion of a sequence at least 90%, at least 95%, at least 98%, at least 99% or 100% identical to positions 321-992 of SEQ ID NO: 1 or the complement thereof.

Optionally, the assay for indicating the presence of a nucleotide sequence corresponding to at least a portion of the rep gene comprises detecting at least a portion of a sequence encoding a Rep52 and/or a Rep40 protein or the complement thereof. Optionally, the assay for indicating the presence of a nucleotide sequence corresponding to at least a portion of the rep gene comprises detecting at least a portion of a sequence corresponding to positions 993-2252 of the AAV2 genome set forth in SEQ ID NO: 1, or the complement thereof. Optionally, the assay for indicating the presence of a nucleotide sequence corresponding to at least a portion of the rep gene comprises detecting at least a portion of a sequence at least 90%, at least 95%, at least 98%, at least 99% or 100% identical to positions 993-2252 of SEQ ID NO: 1 or the complement thereof.

Different serotypes of AAV have Rep proteins having different amino acid sequences. A gene encoding any (set of) Rep protein(s) may be detected in the methods of the present invention—typically the skilled person will be aware of which Rep genes were used in the production of the AAV particles which are to be detected, and may adapt the methods of the present invention to detect at least a portion of a rep gene which encodes a Rep protein of the appropriate serotype. The rep gene may encode a Rep protein of a native Rep protein expressed in AAV of a certain serotype. Alternatively, the rep gene may be a non-natural rep gene, for example, a rep gene engineered to alter the relative levels of expression of the four Rep proteins relative to a natural rep gene. Engineered rep genes are particularly advantageous in the context of producing AAV particles for use in gene therapy applications, as engineering the rep gene that is used may improve the yield of the AAV particles and/or may result in the AAV particles having fewer impurities and/or empty viral particles. In this context, the skilled person would be able to identify regions of a rep gene of an AAV serotype other than AAV2 which correspond to the exemplary rep gene sequence of AAV2 presented herein within the AAV2 genome represented by SEQ ID NO: 1 by performing a suitable alignment as described elsewhere herein, and optionally design suitable primers for detecting the rep gene accordingly.

Optionally, the rep gene encodes a Rep protein from a serotype selected from AAV 1, 2, 3A, 3B, 4, 5, 6, 7, 8, 9, 10, 11, 12, or 13 or any hybrid thereof. Optionally, the rep gene is an engineered Rep gene. Optionally, the rep gene is an engineered rep gene as disclosed in WO 2020/208379 or WO 2022/079429.

Optionally, the assay for indicating the presence of a nucleotide sequence corresponding to at least a portion of the cap gene comprises detecting at least a portion of a sequence encoding a VP1 protein, or the complement thereof. Optionally the assay for indicating the presence of a nucleotide sequence corresponding to at least a portion of the cap gene comprises detecting at least a portion of a sequence corresponding to nucleotides 2203-4407 of the AAV2 genome set forth in SEQ ID NO: 1, or the complement thereof. Optionally, the assay for indicating the presence of a nucleotide sequence corresponding to at least a portion of the cap gene comprises detecting at least a portion of a sequence at least 80%, at least 85%, at least 90%, at least 95%, at least 98%, at least 99% or 100% identical to nucleotides 2203-4407 of SEQ ID NO: 1, or the complement thereof. Optionally, the assay for indicating the presence of a nucleotide sequence corresponding to at least a portion of the cap gene comprises detecting at least a portion of a sequence encoding the portion of the cap gene unique to the VP1 protein, or the complement thereof. Optionally the assay for indicating the presence of a nucleotide sequence corresponding to at least a portion of the cap gene comprises detecting at least a portion of a sequence corresponding to nucleotides 2203-2613 of the AAV2 genome set forth in SEQ ID NO: 1, or the complement thereof. Optionally, the assay for indicating the presence of a nucleotide sequence corresponding to at least a portion of the cap gene comprises detecting at least a portion of a sequence at least 80%, at least 85%, at least 90%, at least 95%, at least 98%, at least 99% or 100% identical to nucleotides 2203-2613 of SEQ ID NO: 1, or the complement thereof.

Optionally, the assay for indicating the presence of a nucleotide sequence corresponding to at least a portion of the cap gene comprises detecting at least a portion of a sequence encoding a VP2 protein, or the complement thereof. Optionally, the assay for indicating the presence of a nucleotide sequence corresponding to at least a portion of the cap gene comprises detecting at least a portion of a sequence corresponding to positions 2614-4407 of the AAV2 genome set forth in SEQ ID NO: 1, or the complement thereof. Optionally, the assay for indicating the presence of a nucleotide sequence corresponding to at least a portion of the cap gene comprises detecting at least a portion of a sequence at least 80%, at least 85%, at least 90%, at least 95%, at least 98%, at least 99% or 100% identical to nucleotides 2614-4407 of SEQ ID NO: 1, or the complement thereof.

Optionally, the assay for indicating the presence of a nucleotide sequence corresponding to at least a portion of the cap gene comprises detecting at least a portion of a sequence encoding a VP3 protein, or the complement thereof. Optionally, the assay for indicating the presence of a nucleotide sequence corresponding to at least a portion of the cap gene comprises detecting at least a portion of a sequence corresponding to positions 2809-4407 of the AAV2 genome set forth in SEQ ID NO: 1, or the complement thereof. Optionally, the assay for indicating the presence of a nucleotide sequence corresponding to at least a portion of the cap gene comprises detecting at least a portion of a sequence at least 80%, at least 85%, at least 90%, at least 95%, at least 98%, at least 99% or 100% identical to nucleotides 2809-4407 of SEQ ID NO: 1, or the complement thereof.

Different serotypes of AAV have Cap proteins having different amino acid sequences. A cap gene encoding any (set of) Cap protein(s) may be detected in the methods of the present invention—typically the skilled person will be aware of the serotype of the AAV particles which are to be detected, and may adapt the methods of the present invention to detect at least a portion of a cap gene which encodes a Cap protein of the appropriate serotype. The cap gene may encode a Cap protein of a native Cap protein expressed in AAV of a certain serotype. Alternatively, the cap gene may encode a Cap protein which is non-natural, for example, an engineered Cap protein, which is designed to comprise a sequence different to that of a native AAV Cap protein (non-natural Cap proteins are particularly advantageous in the context of gene therapy applications, as it is possible that fewer potential patients have levels of antibodies that prevent transduction by AAV comprising non-natural Cap proteins, relative to native capsids). In this context, the skilled person would be able to identify regions of a cap gene of an AAV serotype other than AAV2 which correspond to the exemplary cap gene sequence of AAV2 presented herein within the AAV2 genome represented by SEQ ID NO: 1 by performing a suitable alignment as described elsewhere herein, and optionally design suitable primers for detecting the cap gene accordingly.

Optionally, the cap gene encodes a Cap protein with AAV serotype 1, 2, 3A, 3B, 4, 5, 6, 7, 8, 9, 10, 11, 12, or 13 or any hybrid thereof. Optionally, the cap gene encodes a Cap protein with AAV serotype 2, 5, 8, and 9. Optionally, the cap gene encodes the Cap proteinLK03, rh74, rhl0 or Mut C (WO2016/181123; WO2013/029030; WO2017/096164). Optionally, the cap gene encodes a Cap protein with AAV serotype 2, 5, 8, 9, or the Cap protein Mut C (SEQ ID NO: 3 from WO2016/181123). Optionally, the cap gene encodes the Cap protein Mut C (SEQ ID NO: 3 from WO2016/181123).

It is within the abilities of the person skilled in the art to determine whether a particular (test) stretch of nucleotides “corresponds to” a particular portion of a rep gene or a cap gene stretch of nucleotides in a different serotype of AAV. All that is required is that the person skilled in the art align the test stretch of nucleotides with the genome of the reference serotype (e.g. the AAV2 genome having the sequence SEQ ID NO: 1) using a suitable alignment algorithm such as the EMBOSS Needle Pairwise Sequence Alignment tool described above and determine which region of the alternative amino acid sequence aligns to the specified positions in the genome of the reference serotype. For example, if the test stretch of nucleotides has at least 80% identity with a contiguous stretch of nucleotides of the same length in e.g. SEQ ID NO: 1, the test stretch corresponds to that contiguous stretch of nucleotides in the genome of the reference serotype.

By way of representative example, the nucleotide sequence set forth in SEQ ID NO: 11 encodes the MutC capsid protein (SEQ ID NO: 3 from WO2016/181123). Primers for the detection of nucleotide sequences encoding MutC capsid proteins are used in the Examples below. Nucleotides 2203-4407 of SEQ ID NO: 1 encoding the AAV2 VP1 capsid protein correspond to nucleotides 1-2208 of SEQ ID NO: 11. Nucleotides 2614-4407 of SEQ ID NO: 1 encoding the AAV2 VP2 capsid protein correspond to nucleotides 412-2208 of SEQ ID NO: 11. Nucleotides 2809-4407 of SEQ ID NO: 1 encoding the AAV2 VP3 capsid protein correspond to nucleotides 607-2208 of SEQ ID NO: 11. The person skilled in the art would be able to design corresponding primers and probes suitable for the detection of portions of the AAV2 cap gene of SEQ ID NO: 1 to the exemplary primers and probes used in the Examples herein.

Optionally, two or more nucleotide sequences corresponding to at least portions of a rep gene (or nucleotide sequences corresponding to at least a portion of each of two or more rep genes) and/or two or more nucleotide sequences corresponding to at least portions of a cap gene (or nucleotide sequences corresponding to at least a portion of each of two or more cap genes) may be detected simultaneously.

Optionally, an assay may be performed for indicating the presence of one or more additional nucleotide sequences within each of the plurality of compartments. For example, one or more encapsidated nucleic acid impurities may be detected, quantified and/or sized in addition to assaying for the presence of a nucleotide sequence corresponding to at least a portion of a rep gene and a nucleotide sequence corresponding to at least a portion of a cap gene. The methods of the invention may be extended to look at multiple targets in a sample (i.e. multiplexed methods), such as at least 3, at least 4, at least 5, at least 6, at least 7, at least 8, at least 9, at least 10, at least 11 or at least 12 different targets in a sample.

Optionally, the methods of the invention may further comprise performing an assay for indicating the presence of a nucleotide sequence corresponding to a portion of an ITR within each of the plurality of compartments.

Optionally, at least a portion of an inverted terminal repeat (ITR) may be detected and/or quantified in addition to assaying for the presence of a nucleotide sequence corresponding to at least a portion of a rep gene and a nucleotide sequence corresponding to at least a portion of a cap gene. More particularly, the methods of the invention may comprise detecting any compartments and/or determining the number of compartments, within the plurality of compartments, that comprise a nucleotide sequence corresponding to at least a portion of an ITR.

Optionally, step (c) of the methods of the invention may comprise performing assays for indicating the presence of a nucleotide sequence corresponding to at least a portion of a rep gene, the presence of a nucleotide sequence corresponding to at least a portion of a cap gene, and a nucleotide sequence corresponding to at least a portion of an ITR.

Optionally, the assay for indicating the presence of a nucleotide sequence corresponding to at least a portion of an ITR comprises an ITR PCR assay. Optionally, the ITR PCR assay utilises forward and reverse PCR primers which are capable of hybridising to at least a portion of an ITR sequence. Optionally, the forward and/or reverse PCR primers are capable of hybridising to at least a portion of a sequence corresponding to nucleotides 1-145 of the AAV2 genome set forth in SEQ ID NO: 1, or at least a portion of a sequence complementary thereto. Optionally, the forward PCR primer is capable of hybridising to the reverse complement of at least a portion of a sequence corresponding to nucleotides 1-145 of the AAV2 genome set forth in SEQ ID NO: 1, and/or the reverse PCR primer is capable of hybridising to at least a portion of a sequence corresponding to nucleotides 1-145 of the AAV2 genome set forth in SEQ ID NO: 1.

Optionally, the at least a portion of an ITR may be detected in combination with assaying for the presence of the nucleotide sequence corresponding to the at least a portion of a rep gene and the nucleotide sequence corresponding to the at least a portion of a cap gene.

Optionally, step (d) of the methods of the invention may comprise detecting any compartments, within the plurality of compartments, or determining the number of compartments that comprise, as indicated by the assays of step (c):
i) a nucleotide sequence corresponding to the at least a portion of the rep gene but do not comprise a nucleotide sequence corresponding to the at least a portion of the cap gene or a nucleotide sequence corresponding to the at least a portion of the ITR;
ii) a nucleotide sequence corresponding to the at least a portion of the cap gene but do not comprise the nucleotide sequence corresponding to the at least a portion of the rep gene or a nucleotide sequence corresponding to the at least a portion of the ITR;
iii) a nucleotide sequence corresponding to the at least a portion of an ITR but not a nucleotide sequence corresponding to the at least a portion of the cap gene or a nucleotide sequence corresponding to the at least a portion of the rep gene;
iv) a nucleotide sequence corresponding to the at least a portion of the rep gene and a nucleotide sequence corresponding to the at least a portion of the ITR but not a nucleotide sequence corresponding to the at least a portion of the cap gene;
v) a nucleotide sequence corresponding to the at least a portion of the cap gene and a nucleotide sequence corresponding to the at least a portion of the ITR but do not comprise a nucleotide sequence corresponding to the at least a portion of the rep gene;
vi) a nucleotide sequence corresponding to the at least a portion of the rep gene and a nucleotide sequence corresponding to the at least a portion of the cap gene but do not comprise a nucleotide sequence corresponding to the at least a portion of the ITR; and
vii) a nucleotide sequence corresponding to the at least a portion of the rep gene, a nucleotide sequence corresponding to the at least a portion of the cap gene, and a nucleotide sequence corresponding to the at least a portion of the ITR;

The present invention thus provides methods for detecting and/or quantifying rAAV particles in a sample comprising recombinant adeno-associated virus (rAAV) particles which may be pseudo-wild type rcAAV particles. The detection of compartments that comprise a nucleotide sequence corresponding to the at least a portion of the rep gene, a nucleotide sequence corresponding to the at least a portion of the cap gene and a nucleotide sequence corresponding to the at least a portion of the ITR indicates that the sample comprises rAAV particles which may be pseudo-wild type rcAAV particles. Determining the number of compartments that comprise a nucleotide sequence corresponding to the at least a portion of the rep gene, a nucleotide sequence corresponding to the at least a portion of the cap gene, and a nucleotide sequence corresponding to the at least a portion of an ITR indicates the quantity of rAAV in the sample which may be pseudo-wild type rcAAV particles.

The methods of the invention also comprise detecting compartments which comprise a nucleotide sequence corresponding to at least a portion of one or more ITRs, a nucleotide sequence corresponding to at least a portion of a rep gene and a nucleotide sequence corresponding to at least a portion of a cap gene. Such compartments may be considered to represent compartments which comprise an rAAV that may be pseudo-wild type rcAAV particles.

The nucleotide sequences corresponding to at least a portion of an ITR, at least a portion of a rep gene and at least a portion of a cap gene may be encapsidated in a single rAAV particle. The present invention may, therefore, be considered to provide methods for detecting a nucleotide sequence corresponding to at least a portion of an ITR, a nucleotide sequence corresponding to at least a portion of a rep gene and a nucleotide sequence corresponding to at least a portion of a cap gene which may be encapsidated in the same rAAV particle. Put another way, the present invention provides methods for detecting and/or quantifying rAAV particles which may comprise a nucleotide sequence corresponding to at least a portion of an ITR, a nucleotide sequence corresponding to at least a portion of a rep gene and a nucleotide sequence corresponding to at least a portion of a cap gene (which may be linked or which may be co-packaged). As discussed above, such rAAV particles may be pseudo-wild type rcAAV particles. The detection of compartments that comprise a nucleotide sequence corresponding to the at least a portion of the rep gene, a nucleotide sequence corresponding to the at least a portion of the cap gene and a nucleotide sequence corresponding to the at least a portion of the ITR indicates that the sample comprises rAAV particles that may comprise a nucleotide sequence corresponding to at least a portion of an ITR, a nucleotide sequence corresponding to at least a portion of a rep gene and a nucleotide sequence corresponding to at least a portion of a cap gene. Determining the number of compartments that comprise a nucleotide sequence corresponding to the at least a portion of the rep gene, a nucleotide sequence corresponding to the at least a portion of the cap gene, and a nucleotide sequence corresponding to the at least a portion of an ITR indicates the quantity of rAAV particles which comprise a nucleotide sequence corresponding to at least a portion of an ITR, a nucleotide sequence corresponding to at least a portion of a rep gene and a nucleotide sequence corresponding to at least a portion of a cap gene, and which may be pseudo-wild type rcAAV particles.

Detecting all of a nucleotide sequence corresponding to at least a portion of an ITR, a nucleotide sequence corresponding to at least a portion of a rep gene and a nucleotide sequence corresponding to at least a portion of a cap gene within a compartment may indicate that the ITR, rep and cap nucleotide sequences are encapsidated within a single rAAV particle, particularly where partitioning step (b) is carried in such a way as to minimise the number of compartments comprising two or more AAV particles which comprise at least one of a portion of an ITR, the rep gene or the cap gene. Detecting all of a nucleotide sequence corresponding to at least a portion of an ITR, a nucleotide sequence corresponding to at least a portion of a rep gene and a nucleotide sequence corresponding to at least a portion of a cap gene within a compartment may indicate that the compartment (and thus the sample) comprises an rAAV particle which comprises ITR, rep and cap nucleotide sequences (i.e. that ITR, rep and cap nucleotide sequences are encapsidated within a single rAAV particle). Certainly, detecting compartments which comprise a nucleotide sequence corresponding to at least a portion of an ITR, a nucleotide sequence corresponding to at least a portion of a rep gene and a nucleotide sequence corresponding to at least a portion of a cap gene may still be indicative of the presence (or in quantitative methods, the quantity) of rAAV particles in the sample which comprise a nucleotide sequence corresponding to at least a portion of an ITR, a nucleotide sequence corresponding to at least a portion of a rep gene and a nucleotide sequence corresponding to at least a portion of a cap gene, and thus have utility as methods for assessing the quality of a sample comprising rAAV.

A nucleotide sequence corresponding to at least a portion of an ITR, a nucleotide sequence corresponding to at least a portion of a rep gene and a nucleotide sequence corresponding to at least a portion of a cap gene which are present within a compartment may be linked, or may be unlinked. “Linked” nucleotide sequences are nucleotide sequences which are covalently attached, i.e. which are provided as a single nucleic acid molecule. By way of example, the AAV genome (a single-stranded DNA genome) comprises a 5′ ITR, a rep gene, a cap gene and a 3′ ITR which are linked.

The present invention may therefore be considered to provide methods for detecting nucleotide sequences corresponding to at least a portion of an ITR, at least a portion of a rep gene and/or at least a portion of a cap gene which may be linked (i.e. linked ITR-rep-cap species) in a sample comprising rAAV particles. As discussed above, such linked sequences may be encapsidated in a single rAAV particle, and such rAAV particles may be pseudo-wild type rAAV particles and/or may be replication competent (i.e. may be pseudo-wild type rcAAV particles). The detection of compartments that comprise a nucleotide sequence corresponding to the at least a portion of the rep gene, a nucleotide sequence corresponding to the at least a portion of the cap gene and a nucleotide sequence corresponding to the at least a portion of the ITR indicates that the sample comprises a nucleotide sequence corresponding to at least a portion of an ITR, a nucleotide sequence corresponding to at least a portion of a rep gene and a nucleotide sequence corresponding to at least a portion of a cap gene which may be linked. Determining the number of compartments that comprise a nucleotide sequence corresponding to the at least a portion of the rep gene, a nucleotide sequence corresponding to the at least a portion of the cap gene, and a nucleotide sequence corresponding to the at least a portion of an ITR indicates the quantity of nucleotide sequences corresponding to at least a portion of an ITR at least a portion of a rep gene and at least a portion of a cap gene which may be linked.

Optionally, the at least a portion of an ITR may be detected separately from assaying for the presence of the nucleotide sequence corresponding to the at least a portion of a rep gene and the nucleotide sequence corresponding to the at least a portion of a cap gene. However, as rAAV particles for use in therapy (e.g. for gene therapy) typically comprise a vector genome which comprises the desired “payload” flanked by ITR sequences, a significant proportion of the rAAV particles in a sample may comprise ITR sequences. Thus, where the at least a portion of the ITR is detected separately from assaying for the presence of the nucleotide sequence corresponding to at least a portion of a rep gene and the nucleotide sequence corresponding to the at least a portion of a cap gene, the separate step of detecting the at least portion of the ITR typically comprises assaying for the presence of a nucleotide sequence corresponding to at least a portion of the ITR in combination with assaying for the presence of a nucleotide sequence corresponding to at least a portion of a rep gene or a nucleotide sequence corresponding to at least a portion of a cap gene.

Optionally, step (d) of the methods of the invention may comprise:
(d)(1) detecting any compartments, within the plurality of compartments, or determining the number of compartments that comprise, as indicated by the assays of step (c):
   i) a nucleotide sequence corresponding to the at least a portion of the rep gene but do not comprise a nucleotide sequence corresponding to the at least a portion of the cap gene;
   ii) a nucleotide sequence corresponding to the at least a portion of the cap gene but do not comprise the nucleotide sequence corresponding to the at least a portion of the rep gene; and
   iii) a nucleotide sequence corresponding to the at least a portion of the rep gene and a nucleotide sequence corresponding to the at least a portion of the cap gene; and
(d)(2) detecting any compartments, within the plurality of compartments, or determining the number of compartments that comprise, as indicated by the assays of step (c)
   i) a nucleotide sequence corresponding to the at least a portion of an ITR and a nucleotide sequence corresponding to the at least a portion of the rep gene or a nucleotide sequence corresponding to the at least a portion of the cap gene.

Whilst detecting compartments, within the plurality of compartments, which comprise a nucleotide corresponding to at least a portion of a rep gene and a nucleotide corresponding to at least a portion of a cap gene, and detecting compartments, within the plurality of compartments, which comprise at least a portion of an ITR and a nucleotide sequence corresponding to at least a portion of a rep gene or a nucleotide sequence corresponding to at least a portion of a cap gene may not directly detect compartments which comprise all of a nucleotide sequence corresponding to at least a portion of an ITR, a nucleotide sequence corresponding to at least a portion of a rep gene and a nucleotide sequence corresponding to at least a portion of a cap gene, since rAAV particles are distributed amongst the compartments at random, detecting compartments, within the plurality of compartments, which comprise a nucleotide corresponding to at least a portion of a rep gene and a nucleotide corresponding to at least a portion of a cap gene, and detecting compartments, within the plurality of compartments, which comprise at least a portion of an ITR and a nucleotide sequence corresponding to at least a portion of a rep gene or a nucleotide sequence corresponding to at least a portion of a cap gene may still indirectly indicate that the sample comprises rAAV particles which may comprise all of these nucleotide sequences. Similarly, determining the number of compartments, within the plurality of compartments, which comprise a nucleotide corresponding to at least a portion of a rep gene and a nucleotide corresponding to at least a portion of a cap gene, and determining the number of compartments, within the plurality of compartments, which comprise at least a portion of an ITR and a nucleotide sequence corresponding to at least a portion of a rep gene or a nucleotide sequence corresponding to at least a portion of a cap gene may indicate the quantity of compartments which comprise all of these sequences.

Thus, detecting compartments, within the plurality of compartments, which comprise a nucleotide corresponding to at least a portion of a rep gene and a nucleotide corresponding to at least a portion of a cap gene, and detecting compartments, within the plurality of compartments, which comprise at least a portion of an ITR and a nucleotide sequence corresponding to at least a portion of a rep gene or a nucleotide sequence corresponding to at least a portion of a cap gene allows rAAV particles in a sample comprising recombinant adeno-associated virus (rAAV) (i) which may be pseudo-wild type rcAAV particles, (ii) which may comprise a nucleotide sequence corresponding to at least a portion of an ITR, a nucleotide sequence corresponding to at least a portion of a rep gene and a nucleotide sequence corresponding to a cap gene, and/or (iii) which may comprise a nucleotide sequence which comprise to at least a portion of an ITR, a nucleotide sequence which corresponds to at least a portion of a rep gene and a nucleotide sequence which comprise at least a portion of a cap gene which are linked, to be detected and/or quantified.

Optionally, at least a portion of a 5′ ITR and/or at least a portion of a 3′ ITR may be detected and/or quantified in addition to assaying for the presence of a nucleotide sequence corresponding to at least a portion of a rep gene and a nucleotide sequence corresponding to at least a portion of a cap gene.

Thus, optionally, step (c) of the methods of the invention may comprise performing assays for indicating the presence of a nucleotide corresponding to at least a portion of a rep gene, the presence of a nucleotide sequence corresponding to at least a portion of a cap gene, a nucleotide sequence corresponding to at least a portion of a 5′ ITR and/or a nucleotide sequence corresponding to at least a portion of a 3′ ITR.

The at least a portion of a 5′ ITR and/or the at least a portion of a 3′ ITR may be detected in combination with assaying for the presence of the nucleotide sequence corresponding to the at least a portion of a rep gene and the nucleotide sequence corresponding to the at least a portion of a cap gene, and step (d) of the methods of the invention may be adapted to detect any compartments, within the plurality of compartments, or determine the number of compartments that comprise a nucleotide sequence corresponding to the at least a portion of the 5′ ITR and/or a nucleotide sequence corresponding to the at least a portion of the 3′ ITR, for example by adapting steps (dxiv), (v) and (vii) outlined above to detect and/or determine the number of compartments which comprise both a nucleotide sequence corresponding to at least a portion of a 5′ ITR and/or a nucleotide sequence corresponding to at least a portion of a 3′ ITR.

The at least a portion of a 5′ ITR and/or the at least a portion of a 3′ ITR may alternatively be detected separately from assaying for the presence of the nucleotide sequence corresponding to the at least a portion of a rep gene and the nucleotide sequence corresponding to the at least a portion of a cap gene, and step (d) of the methods of the invention may be adapted to detect any compartments, within the plurality of compartments, and/or determine the number of compartments that comprise a nucleotide sequence corresponding to the at least a portion of the 5′ ITR and/or a nucleotide sequence corresponding to the at least a portion of the 3′ ITR, for example by adapting step (d) to comprise parts (d)(1) and (d)(2), and in (d)(2) detecting any compartments, within the plurality of compartments, or determining the number of compartments that comprise a nucleotide sequence corresponding to the at least a portion of a 5′ ITR and a nucleotide sequence corresponding to the at least a portion of the rep gene or a nucleotide sequence corresponding to at least a portion of the cap gene, and detecting any compartments, within the plurality of compartments, or determining the number of compartments that comprise a nucleotide sequence corresponding to the at least a portion of a 3′ ITR and a nucleotide sequence corresponding to the at least a portion of the rep gene or a nucleotide sequence corresponding to at least a portion of the cap gene.

Typically, a sample comprising rAAV may be obtained from any suitable production system, such as an AAV production system in which a given producer cell (such as a mammalian producer cell or an insect cell line) is used to produce rAAV. For example, a mammalian producer cell may comprise wild type Adenovirus 5 helper genes encoding E2A, E4 and VA RNA I and IL, i.e., the adenovirus helper genes. The mammalian producer cells may be HEK 293 cells, HEK 293T cells, HEK 293 SF cells, HEK 293-F cells, HEK293EBNA cells, HEK 293 derived cells, CHO cells, HeLa cells, HeLa S3 cells, Cap cells, CAP-T cells, AGE1.CR cells, Per.C6 cells, Cl 39 cells, EB66 cells, BHK cells, COS cells, Vero cells or A549 cells. Optionally, the sample comprising rAAV may be obtained from a ‘split-plasmid’ AAV production system, wherein the rep and cap functions are provided on separate plasmids. Examples of such systems are provided in WO2020/208379 and WO2022/079429, the contents of which are hereby incorporated by reference in their entirety.

Optionally, a sample comprising rAA V may be obtained from an rcAAV assay described above. In other words, the sample may be obtained from an assay comprising transducing suitable cells, e.g. HEK293 or HeLa cells, with rAAV in the presence of a helper virus such as adenovirus following a number of rounds (e. g. two or three rounds) of viral amplification, i.e. as a read-out for a classical cell-based rcAAV assay. Optionally, samples may be obtained after two or more different rounds of viral amplification (e.g. a first sample may be obtained after two rounds of viral amplification and a second sample may be obtained after three rounds of viral amplification) and one of the methods of the invention may be used to characterise the rAAV particles after a step of viral amplification or after each round of viral amplification, whereby the detection of or an increase in the number of rAAV particles which may be pseudo-wild type rAAV particles, which may comprise a nucleotide sequence corresponding to at least a portion of a rep gene and a nucleotide sequence corresponding to at least a portion of a cap gene, and/or which comprise at least a portion of a rep gene and at least a portion of a cap gene which may be linked between subsequent rounds of viral amplification may indicate that the rAAV produced by the producer system may comprise rcAAV. Advantageously, employing the methods of the invention in this way may enhance the sensitivity of the methods of the invention. Advantageously, employing the methods of the invention in this way may enhance the specificity of cell-based rcAAV assays and reduce the number of false positive signals, because rep only or cap only containing particles can be distinguished from particles comprising both rep and cap.

Thus, according to one embodiment of the invention, the method may comprise:
(i) contacting a transduceable cell with rAAV particles in the presence of a helper virus;
(ii) incubating the cell for a period of time sufficient for AAV replication to occur;
(iii) harvesting rAAV particles from the cell;
(iv) repeating steps (i)-(iii) one or optionally more than one times to provide a sample comprising rAAV particles; and
(v) carrying out any of the methods described herein to detect and/or quantify the rAAV particles which may be pseudo-wild type, comprise a nucleotide sequence corresponding to at least a portion of a rep gene and a nucleotide sequence corresponding to at least a portion of a cap gene or comprise nucleotide sequences corresponding to at least a portion of a rep gene and at least a portion of a cap gene which may be linked.

Steps (i)-(iii) may represent a viral amplification step in such methods.

Notably, these assays generate rAAV particles which are replication competent, and thus which comprise ITRs. These assays may, therefore, be considered to represent means for indicating the presence of a nucleotide sequence corresponding to at least a portion of an ITR. The rAAV particles may be assayed for the presence of a nucleotide sequence corresponding to at least a portion of a rep gene and a nucleotide sequence corresponding to at least a portion of a cap gene by any of the methods of the invention. This provides alternative means e.g. for determining whether a sample comprises rAAV particles which may be pseudo-wild type rcAAV particles (since the rcAAV assay indirectly indicates the presence of the ITRs, since efficient packaging of the vector genome requires the presence of at least one ITR).

The sample may then be mixed with the appropriate means for indicating the presence of the desired nucleotide sequences (e.g. primers such as PCR primers), optionally following a dilution step, and then randomly partitioned into compartments, such that typically some compartments contain no rAAV particles and other compartments contain one or more rAAV particle. Optionally, it is not necessary to extract the nucleic acid from the rAAV particles. Optionally, intact rAAV particles are partitioned into the compartments. Thus, optionally, the nucleic acid molecules comprised in the rAAV particles are not extracted (i.e. from the AAV particles) prior to step (a) and/or (b), and/or the rAAV particles are not disrupted prior to steps (a) and/or (b).

However, in some embodiments, the nucleic acid may be extracted from the rAAV particles. Optionally, free nucleic acid molecules (i.e. nucleic acid molecules that had previously been encapsidated within rAAV particles, but which have been extracted from the rAAV particles) are partitioned into the compartments. Thus, optionally, the nucleic acid molecules comprised in the rAAV particles may be extracted (i.e. from the AAV particles) prior to step (a) and/or (b), and/or the rAAV particles may be disrupted prior to steps (a) and/or (b). Extracting the nucleic acid molecules from the rAAV particles may resolve the issue whether the rep and cap sequences are linked, or whether the sequences are merely co-packaged (e.g. within a single rAAV particle).

The assays for indicating the presence of a nucleotide sequence corresponding to at least a portion of the rep gene and the presence of a nucleotide sequence corresponding to at least a portion of the cap gene may then be performed on each of the plurality of compartments. Each compartment thus supports an assay for indicating the presence of a nucleotide sequence corresponding to at least a portion of the rep gene and the presence of a nucleotide sequence corresponding to at least a portion of the cap gene (e.g. amplification assays, such as PCR assays), and in particular embodiments a nucleotide sequence corresponding to at least a portion of the rep gene and the presence of a nucleotide sequence corresponding to at least a portion of the cap gene encapsidated in an rAAV.

The nucleic acids in the compartments are typically amplified (e.g. to the terminal plateau phase of PCR (or end-point)). Means for producing a detectable signal may then be used, and the compartments are then read to determine the frequency of compartments which comprise
i) a nucleotide sequence corresponding to the at least a portion of the rep gene but do not comprise a nucleotide sequence corresponding to at least a portion of the cap gene;
ii) a nucleotide sequence corresponding to the at least a portion of the cap gene but do not comprise the nucleotide sequence corresponding to at least a portion of the rep gene; and
iii) a nucleotide sequence corresponding to the at least a portion of the rep gene and a nucleotide sequence corresponding to the at least a portion of the cap gene (and optionally any negative compartments).

### Sizing Nucleotides Corresponding to at Least a Portion of a Rep Gene or at Least a Portion of a Cap Gene

Optionally, the methods of the invention comprise detecting the presence of a nucleotide corresponding to at least a portion of a rep gene and/or a nucleotide corresponding to at least a portion of a cap gene of a pre-determined size in a sample. Methods for sizing nucleic acid impurities in a sample comprising rAAV particles are provided in GB 2205557.8, the contents of which are incorporated herein by reference.

Optionally, the methods of the invention may comprise detecting the presence of a nucleotide sequence corresponding to the at least a portion of the rep gene having a size selected from one or more of 10 bp-50 bp, 10 bp-100 bp, 10 bp-250 bp, 10 bp-500 bp, 10 bp-1000 bp, 10 bp-2500 bp, 10 bp-5000 bp, 50 bp-100 bp, 50 bp-250 bp, 50 bp-500 bp, 50 bp-1000 bp, 50 bp-1500 bp, 100 bp-250 bp, 100 bp-500 bp, 100 bp-1000 bp, 100 bp-1500 bp, 250 bp-500 bp, 250 bp-1000 bp, 250 bp-1500 bp, 500 bp-1000 bp, 500 bp-1500 bp, or 1000 bp-1500 bp. Optionally, the methods of the invention may comprise detecting the presence of a nucleotide sequence corresponding to the full length of the rep gene. Optionally, the methods of the invention may comprise detecting the presence of a nucleotide sequence corresponding to the rep gene having a size of 1932 bp.

Optionally, the methods of the invention may comprise detecting the presence of a nucleotide sequence corresponding to the at least a portion of the cap gene having a size selected from one or more of 10 bp-50 bp, 10 bp-100 bp, 10 bp-250 bp, 10 bp-500 bp, 10 bp-1000 bp, 10 bp-1500 bp, 10 bp-2000 bp, 50 bp-100 bp, 50 bp-250 bp, 50 bp-500 bp, 50 bp-1000 bp, 50 bp-1500 bp, 50 bp-2000 bp, 100 bp-250 bp, 100 bp-500 bp, 100 bp-1000 bp, 100 bp-1500 bp, 100 bp-2000 bp, 250 bp-500 bp, 250 bp-1000 bp, 250 bp-1500 bp, 250 bp-2000 bp, 500 bp-1000 bp, 500 bp-1500 bp, 500 bp-2000 bp, 1000 bp-1500 bp, 1000 bp-2000 bp or 1500 bp-2000 bp. Optionally, the methods of the invention may comprise detecting the presence of a nucleotide sequence corresponding to the full length of the cap gene. Optionally, the methods of the invention may comprise detecting the presence of a nucleotide sequence corresponding to the cap gene having a size of 2205 bp.

The frequency of compartments in which a nucleotide sequence corresponding to the at least a portion of the rep or cap gene of a pre-determined size is present indicates whether or not said nucleotide sequence of the pre-determined size is present. For example, if said frequency is substantially zero, the nucleotide sequence of the pre-determined size is not encapsidated in the rAAV particles in the sample and the result is negative. A suitable threshold may be set for a positive result. For example, a positive result may be called when the nucleotide sequence corresponding to the at least a portion of the rep or cap gene is present in at least 0.1%, at least 0.5%, at least 1%, at least 5%, at least 10%, at least 25%, at least 50%, at least 75%, at least 80%, at least 85%, at least 90% or at least 95% of the compartments. Further, as said frequency increases, the quantity of the at least a portion of the nucleotide sequence of the pre-determined size that is being detected in the sample increases. This increase may be linear. As such, where a first sample has a higher said frequency than a second sample, the first sample will have a higher quantity of the nucleotide sequence of the pre-determined size than the second sample. Further, duplexing or multiplexing using the one or more further nucleotide sequence(s) will allow the detection of the at least a portion of the nucleotide sequence of more than one predetermined size, such as portions of the same nucleotide sequences of different sizes (e.g. at least a portion of a rep gene or at least a portion of a cap gene) and/or portions of different nucleotide sequences each having different sizes. The pre-determined size may be set by the user by selecting forward and reverse primers in a primer pair that would result in an amplification product of the pre-determined size when the nucleotide sequence is present.

Optionally, the methods of the invention may comprise providing an indication of the size of a nucleotide sequence corresponding to at least a portion of a rep gene and/or of a nucleotide corresponding to at least a portion of a cap gene.

Optionally, the methods of the invention may comprise providing an indication of the size of a nucleotide sequence corresponding to at least a portion of a rep gene, said method comprising performing an assay for indicating the presence of a first nucleotide sequence corresponding to a first portion of a rep gene, a second nucleotide sequence corresponding to a second portion of a rep gene, and optionally one or more further nucleotide sequence(s) corresponding to one or more further portions of a rep gene within each of the plurality of compartments, wherein each of the nucleotide sequences corresponds to a different portion of a rep gene, and obtaining an indication of the size of the nucleotide sequence corresponding to at least a portion of the rep gene based on the number of compartments, within the plurality of compartments, that comprise: the first nucleotide sequence; the second nucleotide sequence; the first and second nucleotide sequences; optionally the one or more further nucleotide sequence(s); optionally the first and the one or more further nucleotide sequence(s); and optionally the first, the second and the one or more further nucleotide sequences.

Optionally, the methods of the invention may comprise providing an indication of the size of a nucleotide sequence corresponding to at least a portion of a cap gene, said method comprising performing an assay for indicating the presence of a first nucleotide sequence corresponding to a first portion of a cap gene, a second nucleotide sequence corresponding to a second portion of a cap gene, and optionally one or more further nucleotide sequence(s) corresponding to one or more further portions of a cap gene within each of the plurality of compartments, wherein each of the nucleotide sequences corresponds to a different portion of a cap gene, and obtaining an indication of the size of the nucleotide sequence corresponding to at least a portion of the cap gene based on the number of compartments, within the plurality of compartments, that comprise: the first nucleotide sequence; the second nucleotide sequence; the first and second nucleotide sequences; optionally the one or more further nucleotide sequence(s); optionally the first and the one or more further nucleotide sequence(s); and optionally the first, the second and the one or more further nucleotide sequences.

Thus, by counting the number of compartments which comprise the respective first and second nucleotide sequences, an indication of the size and/or size range of the nucleotide sequences may be inferred. As each of the first etc. nucleotide sequences correspond to a different portion of a nucleotide sequence (e.g. a rep gene or a cap gene), an abundance of compartments in which a single nucleotide sequence is detected is likely to indicate the presence of a relative smaller nucleic acid sequence in the sample comprising rAAV. In contrast, a higher frequency of compartments in which first and second (and optionally subsequent) nucleotide sequences are detected for a nucleotide sequence (e.g. a rep gene or a cap gene) are detected is likely to indicate the presence of larger nucleotide sequences in the sample comprising rAAV, because for a first and second nucleotide sequence, a longer fragment of the nucleotide sequence would in practice need to be present. Thus, the frequency distribution of compartments in which zero, a first, a first and second, and a first, second and subsequent separate nucleotide sequences is/are detected will provide an indication of the size and size range of the nucleotide sequences in the sample comprising rAAV.

Further, where the first, second and optionally one or more further nucleotide sequence(s) are further apart when aligned to the nucleotide sequence being detected, detection of a larger nucleic acid impurity is possible, compared to when the first, second and optionally one or more further nucleotide sequence(s) are closer together when aligned to the nucleotide sequence being detected. Thus, optionally, the method may be adapted to detect larger or smaller nucleotide sequences. When adapted to detect larger nucleotide sequences, it is possible that the method will not detect smaller nucleotide sequences. When adapted to detect smaller nucleotide sequences, it is possible that the method will not detect larger nucleotide sequences. Thus, the method may be adapted to exclude the detection of either larger or smaller nucleotide sequences. Further, multiplexing using the one or more further nucleotide sequence(s) will allow the detection of both larger and smaller nucleotide sequences simultaneously.

### PCR Assays

Optionally, the assay for indicating the presence of a nucleotide sequence corresponding to at least a portion of the rep gene comprises a PCR assay (a rep PCR assay). Optionally, the assay for indicating the presence of a nucleotide sequence corresponding to at least a portion of the cap gene comprises a PCR assay (a cap PCR assay). Optionally, the assay for indicating the presence of a nucleotide sequence corresponding to at least a portion of the rep gene comprises a rep PCR assay, and the assay for indicating the presence of a nucleotide sequence corresponding to at least a portion of the cap gene comprises a cap PCR assay.

Optionally, the rep PCR assay enables the detection of a nucleotide sequence corresponding to at least a portion of a rep gene. Optionally, the rep PCR assay enables the detection of a nucleotide sequence corresponding to at least a portion of a rep gene as described elsewhere herein. Optionally, the rep PCR assay utilises forward and reverse PCR primers which are capable of hybridising to at least a portion of an AAV rep gene.

Optionally, the rep PCR assay utilises forward and/or reverse PCR primers capable of hybridising to at least a portion of a sequence encoding a Rep78 and/or a Rep68 protein. Optionally, the forward and/or reverse PCR primers are capable of hybridising to at least a part of a sequence corresponding to at least a portion of nucleotides 321-2252 of the AAV2 genome set forth in SEQ ID NO: 1, or at least a portion of a sequence complementary thereto. Optionally, the forward and/or reverse PCR primers are capable of hybridising to at least a portion of a sequence at least 90%, at least 95%, at least 98%, at least 99% or 100% identical to nucleotides 321-2252 of SEQ ID NO: 1, or at least a portion of a sequence complementary thereto.

Optionally, the rep PCR assay utilises forward and/or reverse PCR primers capable of hybridising to a portion of an AAV rep gene that does not encode a Rep52 and/or a Rep40 protein or the complement thereof. Thus, optionally, the forward and/or reverse PCR primers are capable of hybridising to the Rep78/Rep68 unique portion of the AAV rep gene or the complement thereof. Optionally, the forward and/or reverse PCR primers are capable of hybridising to at least a portion of a sequence corresponding to nucleotides 321-992 of the AAV2 genome set forth in SEQ ID NO: 1, or at least a portion of a sequence complementary thereto. Optionally, the forward and/or reverse PCR primers are capable of hybridising to at least a portion of a sequence at least 90%, at least 95%, at least 98%, at least 99% or 100% identical to nucleotides 321-992 of SEQ ID NO: 1 or at least a portion of a sequence complementary thereto.

Optionally, the rep PCR assay utilises a forward PCR primer capable of hybridising to the reverse complement of at least a portion of a sequence corresponding to nucleotides 321-521 of the AAV2 genome set forth in SEQ ID NO: 1. Optionally, the forward PCR primer is capable of hybridising to the reverse complement of at least a portion of a sequence 90%, at least 95%, at least 98%, at least 99% or 100% identical to nucleotides 321-521 of SEQ ID NO: 1.

Optionally, the rep PCR assay utilises a reverse PCR primer capable of hybridising to at least a portion of a sequence corresponding to nucleotides 581-992 of the AAV2 genome set forth in SEQ ID NO: 1. Optionally, the reverse PCR primer is capable of hybridising to at least a portion of a sequence at least 90%, at least 95%, at least 98%, at least 99% or 100% identical to nucleotides 581-992 of SEQ ID NO: 1.

Optionally, the rep PCR assay utilises a forward primer which:
(i) is capable of hybridising to the reverse complement of at least a portion of a sequence corresponding to nucleotides 503-521 of the AAV2 genome set forth in SEQ ID NO: 1, optionally to the reverse complement of a sequence corresponding to nucleotides 503-521 of the AAV2 genome set forth in SEQ ID NO: 1;
(ii) comprises or consists of the sequence [CGACTTTCTGACGGAATGG];
(iii) comprises or consists of a sequence at least 90%, preferably at least 95% identical to [CGACTTTCTGACGGAATGG]; or
(iv) comprises or consists of a sequence that has between 1 and 3 substitutions compared to [CGACTTTCTGACGGAATGG].

Optionally, the rep PCR assay utilises a reverse PCR primer which:
(i) is capable of hybridising to at least a portion of a sequence corresponding to nucleotides 581-599 of the AAV2 genome set forth in SEQ ID NO: 1, optionally to a sequence corresponding to nucleotides 581-599 of the AAV2 genome set forth in SEQ ID NO: 1;
(ii) comprises or consists of the sequence [CACGTGCATGTGGAAGTAG];
(iii) comprises or consists of a sequence at least 90%, preferably at least 95% identical to [CACGTGCATGTGGAAGTAG]; or
(iv) comprises or consists of a sequence that has between 1 and 3 substitutions compared to [CACGTGCATGTGGAAGTAG].

Optionally, the rep PCR assay utilises forward and/or reverse PCR primers capable of hybridising to at least a portion of a sequence encoding a Rep52 and/or a Rep40 protein. Optionally, the forward and/or reverse PCR primers are capable of hybridising to at least a portion of a sequence corresponding to nucleotides 993-2252 of the AAV2 genome set forth in SEQ ID NO: 1, or at least a portion of a sequence complementary thereto.

Optionally, the forward and/or reverse PCR primers are capable of hybridising to at least a portion of a sequence at least 90%, at least 95%, at least 98%, at least 99% or 100% identical to nucleotides 993-2252 of SEQ ID NO: 1 or at least a portion of a sequence complementary thereto.

Optionally, the rep PCR assay utilises a forward PCR primer capable of hybridising to the reverse complement of at least a portion of a sequence corresponding to nucleotides 993-1158 of the AAV2 genome set forth in SEQ ID NO: 1. Optionally, the forward PCR primer is capable of hybridising to the reverse complement of at least a portion of a sequence at least 90%, at least 95%, at least 98%, at least 99% or 100% identical to nucleotides 993-1158 of SEQ ID NO: 1.

Optionally, the rep PCR assay utilises a reverse PCR primer capable of hybridising to at least a portion of a sequence corresponding to nucleotides 1226-2252 of the AAV2 genome set forth in SEQ ID NO: 1. Optionally, the reverse PCR primer is capable of hybridising to at least a portion of a sequence 90%, at least 95%, at least 98%, at least 99% or 100% identical to nucleotides 1226-2252 of SEQ ID NO: 1.

Optionally, the rep PCR assay utilises a forward PCR primer which:
(i) is capable of hybridising to the reverse complement of at least a portion of a sequence corresponding to nucleotides 1139-1158 of the AAV2 genome set forth in SEQ ID NO: 1, optionally to the reverse complement of a sequence corresponding to nucleotides 1139-1158 of the AAV2 genome set forth in SEQ ID NO: 1;
(ii) comprises or consists of the sequence [TATGAGCCTGACTAAAACCG];
(iii) comprises or consists of a sequence at least 90%, preferably at least 95% identical to [TATGAGCCTGACTAAAACCG]; or
(iv) comprises or consists of a sequence that has between 1 and 3 substitutions compared to [TATGAGCCTGACTAAAACCG].

Optionally, the rep PCR assay utilises a reverse PCR primer which:
(i) is capable of hybridising to at least a portion of a sequence corresponding to nucleotides 1226-1245 of the AAV2 genome set forth in SEQ ID NO: 1, optionally to a sequence corresponding to nucleotides 1226-1245 of the AAV2 genome set forth in SEQ ID NO: 1;
(ii) comprises or consists of the sequence [GATCGTACCCGTTTAGTTCC];
(iii) comprises or consists of a sequence at least 90%, preferably at least 95% identical to [GATCGTACCCGTTTAGTTCC]; or
(iv) comprises or consists of a sequence that has between 1 and 3 substitutions compared to [GATCGTACCCGTTTAGTTCC].

Optionally, the cap PCR assay enables the detection of a nucleotide sequence corresponding to at least a portion of a cap gene. Optionally, the cap PCR assay enables the detection of a nucleotide sequence corresponding to at least a portion of a cap gene as described elsewhere herein. Optionally, the cap PCR assay utilises forward and reverse PCR primers which are capable of hybridising to at least a portion of an AAV cap gene.

Optionally, the cap PCR assay utilises forward and/or reverse PCR primers capable of hybridising to at least a portion of a sequence encoding a VP1 capsid protein of an AAV capsid. Optionally, the forward and/or reverse PCR primers are capable of hybridising to at least a portion of a sequence corresponding to nucleotides 2203-4407 of the AAV2 genome set forth in SEQ ID NO: 1, or at least a part of a sequence complementary thereto. Optionally, the forward and/or reverse PCR primers are capable of hybridising to at least a portion of a sequence at least 90%, at least 95%, at least 98%, at least 99% or 100% identical to nucleotides 2203-4407 of SEQ ID NO: 1, or at least a portion of a sequence complementary thereto.

Optionally, the cap PCR assay utilises forward and/or reverse PCR primers capable of hybridising to at least a portion of a sequence encoding a VP1 capsid protein of an AAV capsid that is unique to the VP1 protein. Optionally, the forward and/or reverse PCR primers are capable of hybridising to at least a portion of a sequence corresponding to nucleotides 2203-2613 of the AAV2 genome set forth in SEQ ID NO:1, or at least a part of a sequence complementary thereto. Optionally, the forward/or reverse PCR primers are capable of hybridising to at least a portion of a sequence at least 90%, at least 95%, at least 98%, at least 99% or 100% identical to nucleotides 2203-2613 of SEQ ID NO: 1, or at least a portion of a sequence complementary thereto.

Optionally, the cap PCR assay utilises forward and/or reverse PCR primers capable of hybridising to at least a portion of a sequence encoding a VP2 capsid protein of an AAV capsid. Optionally, the forward and/or reverse PCR primers are capable of hybridising to at least a portion of a sequence corresponding to nucleotides 2614-4407 of the AAV2 genome set forth in SEQ ID NO: 1, or at least a portion of a sequence complementary thereto. Optionally, the forward and/or reverse PCR primers are capable of hybridising to at least a portion of a sequence at least 90%, at least 95%, at least 98%, at least 99% or 100% identical to nucleotides 2614-4407 of SEQ ID NO: 1 or at least a portion of a sequence complementary thereto.

Optionally, the cap PCR assay utilises forward and/or reverse PCR primers capable of hybridising to at least a portion of a sequence encoding a VP3 capsid protein of an AAV capsid. Optionally, the forward and/or reverse PCR primers are capable of hybridising to at least a portion of a sequence corresponding to nucleotides 2809-4407 of the AAV2 genome set forth in SEQ ID NO: 1, or at least a portion of a sequence complementary thereto. Optionally, the forward and/or reverse PCR primers are capable of hybridising to at least a portion of a sequence at least 90%, at least 95%, at least 98%, at least 99% or 100% identical to nucleotides 2809-4407 of SEQ ID NO: 1 or at least a portion of a sequence complementary thereto.

Optionally, the cap PCR assay utilises a forward PCR primer capable of hybridising to the reverse complement of at least a portion of a sequence corresponding to nucleotides 2809-3247 of the AAV2 genome set forth in SEQ ID NO: 1. Optionally, the forward PCR primer is capable of hybridising to the reverse complement of at least a portion of a sequence 90%, at least 95%, at least 98%, at least 99% or 100% identical to nucleotides 2809-3247 of SEQ ID NO: 1.

Optionally, the cap PCR assay utilises a reverse PCR primer capable of hybridising to at least a portion of a sequence corresponding to nucleotides 3307-4407 of the AAV2 genome set forth in SEQ ID NO: 1. Optionally, the reverse PCR primer is capable of hybridising to at least a portion of a sequence at least 90%, at least 95%, at least 98%, at least 99% or 100% identical to nucleotides 3307-4407 of SEQ ID NO: 1.

Optionally, the cap PCR assay utilises a forward PCR primer which:
(i) is capable of hybridising to the reverse complement of at least a portion of a sequence corresponding to positions 3228-3247 of the AAV2 genome set forth in SEQ ID NO: 1, optionally to the reverse complement of a sequence corresponding to positions 3228-3247 of the AAV2 genome set forth in SEQ ID NO: 1;
(ii) comprises or consists of the sequence [GTTTACGGACTCGGAGTATC];
(iii) comprises or consists of a sequence at least 90%, preferably at least 95% identical to [GTTTACGGACTCGGAGTATC]; or
(iv) comprises or consists of a sequence that has between 1 and 3 substitutions compared to [GTTTACGGACTCGGAGTATC].

Optionally, the cap PCR assay utilises a reverse PCR primer which:
(i) is capable of hybridising to at least a portion of a sequence corresponding to nucleotides 3307-3326 of the AAV2 genome set forth in SEQ ID NO: 1, optionally to a sequence corresponding to nucleotides 3307-3326 of the AAV2 genome set forth in SEQ ID NO: 1;
(ii) comprises or consists of the sequence [TACTGAGGGACCATGAAGAC];
(iii) comprises or consists of a sequence at least 90%, preferably at least 95% identical to [TACTGAGGGACCATGAAGAC]; or
(iv) comprises of consists of a sequence that has between 1 and 3 substitutions compared to [TACTGAGGGACCATGAAGAC].

Optionally, a forward PCR primer binds to a sequence a suitable distance from the reverse complement of the sequence to which a reverse PCR primer binds to allow efficient PCR to take place. Optionally, a forward PCR primer binds to a sequence at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp from the reverse complement of the sequence to which a reverse PCR primer binds. In this context, reference to a forward PCR primer binding to a sequence a particular number of bp from the reverse complement of the sequence to which a reverse PCR primer binds means the number of nucleotides in a target nucleic acid molecule (e.g. a rep or a cap gene) between where the 3′ end of the forward PCR primer binds to the target nucleic acid molecule, and where the 3′ end of the reverse PCR primer binds to the reverse complement of the target nucleic acid molecule. In other words, this term refers to the ‘gap’ between where the respective 3′ ends of the forward and reverse PCR primers bind to the target nucleic acid molecule and its reverse complement.

Thus, optionally, the forward PCR primer for the rep PCR assay binds to a sequence at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp from the reverse complement of the sequence to which the reverse PCR primer for the rep PCR assay binds.

Furthermore, optionally, the forward PCR primer for the cap PCR assay binds to a sequence at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or less than 1.8 kb, less than 1.6 kb, less than 1.5 kb, less than 1.4 kb, less than 1.3 kb, less than 1.2 kb, less than 1.1 kb, less than 1.1 kb, less than 900 bp, less than 800 bp, less than 700 bp, less than 600 bp, less than 500 bp, less than 400 bp or less than 300 bp from the reverse complement of the sequence to which the reverse PCR primer for the cap PCR assay binds.

PCR primers may also be designed to generate a suitable size amplification product to allow the amplification product to be detected efficiently in the methods of the present invention. For example, an amplification product may be of a sufficient length to allow a probe to bind to the amplification product in a sensitive and specific manner. Optionally, the amplification product of a PCR assay is at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or wherein the amplification products produced by the rep PCR assay and/or the cap PCR assay are less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp in length.

Thus, optionally, the amplification product produced by the rep PCR assay is at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or wherein the amplification products produced by the rep PCR assay and/or the cap PCR assay are less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp in length.

Furthermore, optionally, the amplification product produced by the cap PCR assay is at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or wherein the amplification products produced by the rep PCR assay and/or the cap PCR assay are less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp in length.

### Nucleic Acid Probes

Herein, the term “nucleic acid probe” is intended to refer to an oligonucleotide which may provide or produce a detectable signal that indicates the presence of a nucleic acid molecule having a particular sequence in a sample. Thus, for example, in the context of the present invention, nucleic acid probes may provide means for producing a detectable signal, and thus may be used to indicate the presence of at least a portion of a rep gene and/or at least a portion of a cap gene in the methods of the invention, thereby indicating which compartments may comprise nucleotide sequences corresponding to at least a portion of a rep gene and/or at least a portion of a cap gene in order to detect rAAV particles which may be replication competent.

Optionally, nucleotide sequences may be detected using nucleic acid probes in the methods of the present invention. In other words, assays for indicating the presence of particular nucleotide sequences may involve the use of nucleic acid probes. Optionally, a nucleotide sequence corresponding to at least a portion of a rep gene may be detected using a nucleic acid probe. Optionally, the assay for indicating the presence of a nucleotide sequence corresponding to at least a portion of the rep gene comprises the use of a nucleic acid probe. Optionally, a nucleotide sequence corresponding to at least a portion of a cap gene may be detected using a nucleic acid probe. Optionally, the assay for indicating the presence of a nucleotide sequence corresponding to at least a portion of the cap gene comprises the use of a nucleic acid probe. Optionally, one or more additional nucleotide sequences (e.g. a nucleotide sequence corresponding to at least a portion of an ITR), may be detected using a nucleic acid probe. Optionally, the assay for indicating the presence of one or more additional nucleotide sequences (e.g. a nucleotide sequence corresponding to at least a portion of an ITR) comprises the use of a nucleic acid probe.

Optionally, the assay for indicating the presence of a nucleotide sequence corresponding to at least a portion of the rep gene comprises the use of a nucleic acid probe for detecting an AAV rep gene. Said nucleic acid probe for detecting an AAV rep gene is capable of hybridising to at least a portion of the AAV rep gene or the complement thereof.

Optionally, the assay for indicating the presence of a nucleotide sequence corresponding to at least a portion of the cap gene comprises the use of a nucleic acid probe for detecting an AAV cap gene. Said nucleic acid probe for detecting an AAV cap gene is capable of hybridising to at least a portion of the AAV cap gene or the complement thereof.

Thus, optionally the assay for indicating the presence of a nucleotide sequence corresponding to at least a portion of the rep gene comprises the use of a nucleic acid probe capable of hybridising to the at least a portion of the AAV rep gene or the complement thereof for detecting an AAV rep gene, and/or the assay for indicating the presence of a nucleotide sequence corresponding to at least a portion of the cap gene comprises the use of a nucleic acid probe capable of hybridising to the at least a portion of the AAV cap gene or the complement thereof for detecting an AAV cap gene.

More particularly, the amplification product(s) of a rep PCR assay and/or a cap PCR assay may optionally be detected using a nucleic acid probe for detecting an AAV rep gene and/or a nucleic acid probe(s) for detecting an AAV cap gene.

Thus, optionally an amplification product of the rep PCR assay is detected using a nucleic acid probe capable of hybridising to at least a portion of the AAV rep gene or the complement thereof for detecting an AAV rep gene, and/or an amplification product of the cap PCR assay is detected using a nucleic acid probe capable of hybridising to at least a portion of the AAV cap gene or the complement thereof for detecting an AAV cap gene.

Optionally, the nucleic acid probe for detecting the AAV rep gene is a nucleic acid probe capable of hybridising to at least a portion of an AAV rep gene or the complement thereof. Optionally, the nucleic acid probe for detecting the AAV rep gene is a nucleic acid probe capable of hybridising to at least a portion of a sequence encoding a Rep protein or the complement thereof.

Optionally, the nucleic acid probe for detecting the AAV rep gene is a nucleic acid probe capable of hybridising to at least a portion of a sequence encoding a Rep78 and/or a Rep68 protein or the complement thereof.

Optionally, the nucleic acid probe for detecting the AAV rep gene is capable of hybridising to at least a portion of sequence corresponding to nucleotides 321-2252 of the AAV2 genome set forth in SEQ ID NO: 1 or the complement thereof. Optionally, the nucleic acid probe for detecting the AAV rep gene is a nucleic acid probe capable of hybridising to at least a portion of a sequence at least 90%, at least 95%, at least 98%, at least 99% or 100% identical to nucleotides 321-2252 of SEQ ID NO: 1, or the complement thereof.

Optionally, the nucleic acid probe for detecting the AAV rep gene is a nucleic acid probe capable of hybridising to a portion of an AAV rep gene that does not encode a Rep52 and/or a Rep40 protein or the complement thereof. Thus, optionally, the nucleic acid probe for detecting the AAV rep gene is a nucleic acid probe capable of hybridising to at least a portion of the Rep78/Rep68 unique portion of the AAV rep gene or the complement thereof. Optionally, the nucleic acid probe for detecting the AAV rep gene is a capable of hybridising to at least a portion of a sequence corresponding to positions 321-992 of the AAV2 genome set forth in SEQ ID NO: 1, or the complement thereof. Optionally, the nucleic acid probe for detecting the AAV rep gene is capable of hybridising to at least a portion of a sequence at least 90%, at least 95%, at least 98%, at least 99% or 100% identical to positions 321-992 of SEQ ID NO: 1 or the complement thereof.

Optionally, the nucleic acid probe for detecting the AAV rep gene:
(i) is capable of hybridising to at least a portion of a sequence corresponding to nucleotides 522-580 of the AAV2 genome set forth in SEQ ID NO: 1, optionally to at least a portion of a sequence corresponding to nucleotides 525-544 of the AAV2 genome set forth in SEQ ID NO: 1, optionally to a sequence corresponding to nucleotides 525-544 of the AAV2 genome set forth in SEQ ID NO: 1;
(ii) comprises or consists of the sequence [TCCGGGGCCTTACTCACACG];
(iii) comprises or consists of a sequence at least 90%, preferably at least 95% identical to [TCCGGGGCCTTACTCACACG];
(iv) comprises or consists of a sequence that has between 1 and 3 substitutions compared to [TCCGGGGCCTTACTCACACG]; or
(v) comprises or consists of a sequence complementary to (i), (ii), (iii) or (iv).

Optionally, the nucleic acid probe for detecting the AAV rep gene is a nucleic acid probe capable of hybridising to at least a portion of a sequence encoding a Rep52 and/or a Rep40 protein or the complement thereof. Optionally, the nucleic acid probe for detecting the AAV rep gene is a nucleic acid probe capable of hybridising to at least a portion of a sequence corresponding to positions 993-2252 of the AAV2 genome set forth in SEQ ID NO: 1, or the complement thereof. Optionally, the nucleic acid probe for detecting the AAV rep gene is a nucleic acid probe capable of hybridising to at least a portion of a sequence at least 90%, at least 95%, at least 98%, at least 99% or 100% identical to positions 993-2252 of SEQ ID NO: 1 or the complement thereof.

Optionally, the nucleic acid probe for detecting the AAV rep gene:
(i) is capable of hybridising to at least a portion of a sequence corresponding to nucleotides 1159-1225 of the AAV2 genome set forth in SEQ ID NO: 1, optionally to at least a portion of a sequence corresponding to nucleotides 1189-1210 of the AAV2 genome set forth in SEQ ID NO: 1, optionally to a sequence corresponding to nucleotides 1189-1210 of the AAV2 genome set forth in SEQ ID NO: 1;
(ii) comprises or consists of the sequence [TGGAGGACATTTCCAGCAATCG];
(iii) comprises or consists of a sequence at least 90%, preferably at least 95% identical to [TGGAGGACATTTCCAGCAATCG];
(iv) comprises or consists of a sequence that has between 1 and 3 substitutions compared to [TGGAGGACATTTCCAGCAATCG]; or
(v) comprises or consists of a sequence complementary to (i), (ii), (iii) or (iv).

Optionally, the nucleic acid probe for detecting the AAV cap gene is a nucleic acid probe capable of hybridising to at least a portion of an AAV cap gene or the complement thereof. Optionally, the nucleic acid probe for detecting the AAV cap gene is a nucleic acid probe capable of hybridising to at least a portion of a sequence encoding a Cap protein or the complement thereof.

Optionally, the nucleic acid probe for detecting the AAV cap gene is a nucleic acid probe capable of hybridising to at least a portion of a sequence encoding a VP1 protein, or the complement thereof. Optionally the nucleic acid probe for detecting the AAV cap gene is capable of hybridising to at least a portion of a sequence corresponding to nucleotides 2203-4407 of the AAV2 genome set forth in SEQ ID NO: 1, or the complement thereof. Optionally, the nucleic acid probe for detecting the AAV cap gene is a nucleic acid probe capable of hybridising to at least a portion of a sequence at least 80%, at least 85%, at least 90%, at least 95%, at least 98%, at least 99% or 100% identical to nucleotides 2203-4407 of SEQ ID NO: 1, or the complement thereof.

Optionally, the nucleic acid probe for detecting the AAV cap gene is a nucleic acid probe capable of hybridising to at least a portion of a sequence encoding a VP3 protein, or the complement thereof. Optionally, the nucleic acid probe for detecting the AAV cap gene is a nucleic acid probe capable of hybridising to at least a portion of a sequence corresponding to positions 2809-4407 of the AAV2 genome set forth in SEQ ID NO: 1, or the complement thereof. Optionally, the nucleic acid probe for detecting the AAV cap gene is a nucleic acid probe capable of hybridising to at least a portion of a sequence at least 80%, at least 85%, at least 90%, at least 95%, at least 98%, at least 99% or 100% identical to nucleotides 2809-4407 of SEQ ID NO: 1, or the complement thereof.

Optionally, the nucleic acid probe for detecting the AAV cap gene:
(i) is capable of hybridising to at least a portion of a sequence corresponding to nucleotides 3248-3306 of the AAV2 genome set forth in SEQ ID NO: 1, optionally to at least a portion of a sequence corresponding to nucleotides 3282-3300 of the AAV2 genome set forth in SEQ ID NO: 1, optionally to a sequence corresponding to nucleotides 3282-3300 of the AAV2 genome set forth in SEQ ID NO: 1;
(ii) comprises or consists of the sequence [TGGAAACGGCGGGAGACAG];
(iii) comprises or consists of a sequence at least 90%, preferably at least 95% identical to [TGGAAACGGCGGGAGACAG];
(iv) comprises or consists of a sequence that has between 1 and 3 substitutions compared to [TGGAAACGGCGGGAGACAG]; or
(v) comprises or consists of a sequence complementary to (i), (ii), (iii) or (iv).

Typically, the nucleic acid probes for detecting the rep and cap gene (and optionally for detecting one or more additional nucleotide sequences, e.g. a nucleotide sequence corresponding to at least a portion of an ITR) may comprise detectable labels. That is to say, the nucleic acid probes may be labelled with a detectable label. The detectable label may be present at the 5′ end or the 3′ end of the probe. Optionally, the detectable label is present at the 5′ end of the probe.

The detectable label may be a fluorescent reporter dye, or “fluorophore”. A “fluorophore” refers to a molecule that emits light of a certain wavelength after having first absorbed light of a specific, but shorter, wavelength, wherein the emission wavelength is always longer than the absorption wavelength.

In order to identify compartments which comprise a sequence corresponding to at least a portion of the rep gene and compartments which comprise a sequence corresponding to at least a portion of the cap gene, the presence of a nucleotide sequence corresponding to at least a portion of a rep gene is indicated by a different signal to the presence of a nucleotide sequence corresponding to at least a portion of a cap gene in the assay that is performed in the methods of the invention. Thus, compartments which comprise a nucleotide sequence corresponding to the at least a portion of the rep gene are detectably distinct from compartments which do not comprise a nucleotide sequence corresponding to the at least a portion of the rep gene but comprise a nucleotide sequence corresponding to the at least a portion of the cap gene and vice-versa. Similarly, compartments which comprise a nucleotide sequence corresponding to the at least a portion of the rep gene and a nucleotide sequence corresponding to the at least a portion of a cap gene are detectably distinct from compartments which do not comprise either nucleotide sequence. Optionally compartments which comprise one or more additional nucleotide sequences, e.g. a nucleotide sequence corresponding to at least a portion of an ITR are also detectably distinct from compartments which do not comprise the additional nucleotide sequence.

Optionally, where more than one nucleic acid probe is used (e.g. where a first nucleic acid probe is used for detecting the rep gene and a second nucleic acid probe is used for detecting a cap gene), at least one of the nucleic acid probes comprises a detectable label (for example, a fluorophore). Optionally, both the first and second nucleic acid probes comprise a detectable label. Optionally where a third or further nucleic acid probe is used for detecting one or more additional nucleotide sequences, e.g. a nucleotide sequence corresponding to at least a portion of an ITR), the third or further nucleic acid probe comprise a detectable label.

Optionally each nucleic acid probe comprises a different detectable label. Optionally, each nucleic acid probe comprises a different fluorophore. Optionally, when more than one fluorophore is used, each fluorophore has a non-overlapping excitation wavelength, and each fluorophore has a non-overlapping emission wavelength. The fluorophore(s) may have an emission wavelength of around 570 nm to around 530 nm, around 560 nm to around 540 nm, around 560 nm, around 556 nm, around 530 nm to around 500 nm, around 520 nm to around 510 nm or around 520 nm. For example, one fluorophore may have an emission wavelength of around 530 nm to around 500 nm, around 520 nm to around 510 nm, or around 520 nm. If present, the other (for example second) fluorophore may have an emission wavelength of around 570 nm to around 530 nm, around 560 nm to around 540 nm, around 560 nm or around 556 nm. The fluorophore may be 6-Carboxyfluorescein (FAM) or hexachloro-fluorescein (HEX). For example when more than one fluorophore is used, the first fluorophore may be FAM and the second fluorophore may be HEX. Alternatively, the first fluorophore may be HEX and the second fluorophore may be FAM.

Optionally, and alternatively, first and second nucleic acid probes may comprise the same detectable label, but a first nucleic acid probe may be labelled with the detectable label at a lower concentration that a second nucleic acid probe. In other words, the first nucleic acid probe may optionally be labelled with the detectable label at a lower stoichiometric ratio than the second nucleic acid probe.

The at least one nucleic acid probe optionally comprises a quenching agent. A “quenching agent” refers to a molecule that accepts energy from a fluorophore in the form of light at a particular wavelength and dissipates this energy either in the form of heat (e.g. proximal quenching) or light of a longer wavelength than emitted from the fluorophore (e.g. FRET quenching). Quenchers generally have a quenching capacity throughout their absorption spectrum, but they perform best close to their absorption maximum. For example, Deep Dark Quencher II absorbs over a large range of the visible spectrum and, consequently, efficiently quenches most of the commonly used fluorophores, especially those emitting at higher wavelengths (like the Cy® dyes). Similarly, the Black Hole Quencher (BHQ) family, such as BHQ1, covers a large range of wavelengths (over the entire visible spectrum and into the near-IR). The BHQ1 quencher is disclosed, for example, in WO 2001/086001. In contrast, Deep Dark Quencher I and Eclipse® Dark Quencher effectively quench the lower wavelength dyes, such as FAM, but do not quench very effectively those dyes that emit at high wavelengths. The at least one nucleic acid probe may comprise a quenching agent at either the 5′ or 3′ end. Preferably, the quenching agent is present at opposite end of the probe to the detectable label. For example, if the detectable label is present at the 5′ end of the probe, the quenching agent is preferably present at the 3′ end of the probe. The quenching agent may be selected from any suitable agent, such as those set out above. Suitably, the quenching agent is BHQ1. Optionally, upon extension of a primer by a polymerase, a probe comprising a fluorophore at its 5′ end and quencher at its 3′ end is cleaved and displaced by the polymerase and the fluorophore is detached from the probe and thus the quenching agent, resulting in the generation of a detectable signal. Optionally the nucleic acid probes for detecting the rep and the cap genes are TaqMan® probes.

### Double-Stranded DNA Intercalating Dyes

Whilst nucleic acid probes may be used to indicate e.g. the presence of a nucleotide sequence corresponding to at least a portion of a rep gene and/or the presence of a nucleotide sequence corresponding to at least a portion of a cap gene, e.g. as a means of producing a detectable signal for the detection of amplification products (e.g. of a rep PCR assay and/or a cap PCR assay), detectable signals (i.e. for the detection of such amplification products) may be produced by other means.

Optionally, the means or producing a detectable signal may be a double-stranded DNA intercalating dye. Thus, optionally, the amplification products produced in assays for indicating the presence of nucleotide sequences corresponding to at least portions of a rep gene and/or a cap gene (and optionally for detecting one or more additional nucleotide sequences, e.g. a nucleotide sequence corresponding to at least a portion of an ITR) may be detected using a double-stranded DNA intercalating dye. Optionally, the amplification products of a rep PCR assay and/or a cap PCR assay may be detected using a double-stranded DNA intercalating dye. Double-stranded DNA intercalating dyes may insert themselves into a DNA double-stranded helix by packing between adjacent pairs of planar bases within the DNA double helix. Upon insertion of the dye into the double helix structure, the dye typically produces a detectable signal that is not produced in the absence of double-stranded DNA, thereby indicating the presence of double-stranded DNA. Thus, detection of the detectable signal allows the detection of double-stranded DNA.

The signal produced by the double-stranded DNA intercalating dye is proportional to the amount of double-stranded DNA that is present. Thus, a greater signal will be generated in a sample comprising more double-stranded DNA than in a sample comprising less double-stranded DNA. Thus, in the methods of the present invention, a compartment that comprises more double-stranded DNA will produce a greater detectable signal than a compartment that comprises less double-stranded DNA. A double-stranded DNA intercalating dye may therefore be of utility in the methods of the present invention if compartments comprising i) a nucleotide sequence corresponding to the at least a portion of the rep gene but do not comprise a nucleotide sequence corresponding to at least a portion of the cap gene; ii) a nucleotide sequence corresponding to the at least a portion of the cap gene but do not comprise the nucleotide sequence corresponding to at least a portion of the rep gene; and/or iii) a nucleotide sequence corresponding to at least a portion of the rep gene and a nucleotide sequence corresponding to the at least a portion of the cap gene may be distinguished on the basis of the amount of double-stranded DNA present in the respective compartments.

Optionally, an assay for indicating the presence of a nucleotide sequence corresponding to at least a portion of a rep gene (e.g. a rep PCR assay) produces an amount of double-stranded DNA (e.g. an amplification product) that is different to the amount of double-stranded DNA (e.g. an amplification product) produced in an assay for indicating the presence of a nucleotide sequence corresponding to at least a portion of a cap gene (e.g. a cap PCR assay). By way of illustrative example, an assay for indicating the presence of a nucleotide sequence corresponding to at least a portion of a rep gene may produce a low amount of double-stranded DNA, whereas an assay for indicating the presence of a nucleotide sequence corresponding to at least a portion of a cap gene may produce an intermediate amount of double-stranded DNA. In such a scenario, use of a double-stranded DNA intercalating dye will produce a low signal for compartments which comprise a nucleotide sequence corresponding to at least a portion of a rep gene, and an intermediate signal for compartments which comprise a nucleotide sequence corresponding to at least a portion of a cap gene. Furthermore, in such a scenario, compartments which comprise both a nucleotide sequence corresponding to at least a portion of a rep gene and a nucleotide sequence corresponding to at least a portion of a cap gene will comprise a high amount of double-stranded DNA (the sum of the amount of double-stranded DNA produced by the assay for indicating the presence of a nucleotide sequence corresponding to at least a portion of a rep gene and the amount of double-stranded DNA produced by the assay for indicating the presence of a nucleotide sequence corresponding to at least a portion of a cap gene), and thus use of a double-stranded DNA dye will produce a high signal for such compartments. Compartments which comprise a nucleotide sequence corresponding to the at least a portion of the rep gene but do not comprise a nucleotide sequence corresponding to at least a portion of the cap gene; a nucleotide sequence corresponding to the at least a portion of the cap gene but do not comprise the nucleotide sequence corresponding to at least a portion of the rep gene; and a nucleotide sequence corresponding to the at least a portion of the rep gene and a nucleotide sequence corresponding to the at least a portion of the cap gene may therefore be detected and distinguished from one-another using a double-stranded DNA intercalating dye.

The assays for indicating the presence of a nucleotide sequence corresponding to at least a portion of a rep gene and the presence of a nucleotide sequence corresponding to at least a portion of a cap gene within each of the plurality of compartments may optionally be configured to produce different amounts of double-stranded DNA.

Optionally, the efficiency of the rep PCR assay may be configured to be different to the efficiency of the cap PCR assay. The “efficiency” of a PCR reaction is defined as the fraction of target molecules that are copied in one PCR cycle. Thus, a PCR reaction with a lower efficiency will produce fewer copies of a target molecule than a PCR reaction with a higher efficiency, for a given number of cycles. Optionally, the rep PCR assay may be configured to be less efficient than the cap PCR assay. Optionally, the cap PCR assay may be configured to be less efficient than the rep PCR assay.

The Tₘ value of PCR primers can affect the efficiency of a PCR reaction. The Tₘ of a primer can be calculated using the following formula:

$${T}_{m} = [ 4 \times ( G + C ) ] + [ 2 \times ( A + T ) ]$$

The Tₘ value of a primer may depend on the length of the primer. Typically, a longer primer may have a higher Tₘ value than a shorter primer. The Tₘ value of a primer may also depend on the G/C content of the primer. Typically, a primer having a high G/C content (i.e. which is complementary to a nucleotide sequence with a high G/C content) may have a higher Tₘ value than a primer having a high A/T primer. It is therefore within the abilities of the skilled person to design primers having desired Tₘ values by adjusting the length and/or G/C content of the primers.

Without wishing to be bound by theory, if the Tₘ PCR primers is lower than the annealing temperature used in a PCR, the PCR primers may anneal poorly to the target nucleotide, and thus the efficiency of the PCR reaction may be reduced. Optionally, the PCR primers capable of hybridising to the rep gene and the PCR primers capable of hybridising to the cap gene have different Tₘ values. Optionally, the PCR primers capable of hybridising to the rep gene have a higher Tₘ value than the PCR primers capable of hybridising to the cap gene. Optionally, the PCR primers capable of hybridising to the cap gene have a higher Tₘ value than the PCR primers capable of hybridising to the rep gene.

As mentioned above, the signal produced by the double-stranded DNA intercalating dye is proportional to the amount of double-stranded DNA that is present. The signal is proportional to the total amount of double-stranded DNA, rather than the copy number of the double-stranded DNA molecules. Thus, for a given copy number, a sample comprising longer double-stranded DNA molecules will produce a greater signal than a sample comprising shorter double-stranded DNA molecules. The assays for indicating the presence of a nucleotide sequence corresponding to at least a portion of a rep gene and the presence of a nucleotide sequence corresponding to at least a portion of a cap gene within each of the plurality of compartments may optionally produce different amounts of double-stranded DNA by producing double-stranded DNA molecules of different lengths. Optionally, the amplification products of the PCR assays to indicate the presence of the rep and the cap gene are of different lengths.

Optionally, the double-stranded DNA intercalating dye is a fluorescent dye. Optionally, the double-stranded DNA intercalating dye is not significantly fluorescent when not bound to (intercalated with) double-stranded DNA. Optionally, the double-stranded DNA intercalating dye becomes fluorescent upon binding to (intercalation with) double-stranded DNA. Optionally, the double-stranded DNA intercalating dye is an EvaGreen® dye.

Partitioning the Sample into a Plurality of Compartments

Optionally, the compartments are oil droplets. Optionally, partitioning the sample into a plurality of compartments comprises emulsifying the sample with droplet generator oil. Optionally, the method of methods of the invention may be carried out using droplet digital PCR.

The methods of the present invention may, therefore, be carried out by “droplet digital PCR” (ddPCR). ddPCR refers to a digital PCR assay that measures absolute quantities by counting nucleic acid molecules encapsulated in a plurality of compartments. These compartments are typically discrete, water-in-oil droplet compartments that support PCR amplification and may be volumetrically defined. Advantageously, ddPCR is not subject to the amplification bias of other PCR techniques, and does not require a standard curve to be generated. Rather, ddPCR provides an end-point measurement that provides the ability to quantify nucleic acids, such as nucleotide sequences corresponding to at least a portion of a rep gene and/or at least a portion of a cap gene in a sample comprising rAAV.

Droplet digital PCR may be performed using any platform that performs a digital PCR assay that measures absolute quantities by counting nucleic acid molecules encapsulated in the plurality of compartments. The strategy for droplet digital PCR may be summarised as follows: a sample, such as a sample comprising rAAV, is obtained. The sample is then optionally diluted before a step of partitioning into compartments (z.e. separate reaction compartments that are typically water-in-oil compartments), and typically into thousands to millions of compartments. Typically, each compartment contains one or no copies of the nucleic acid molecule(s) to be detected. A ddPCR assay can then be performed on each of the plurality of compartments. Positive compartments, which contain an amplification product or amplification products of interest are then detected or counted (i.e. the number of positive compartments may be detected). Optionally, negative compartments, which do not contain an amplification product, may also detected or counted.

Alternatively, the sample may be partitioned into a plurality of compartments by partitioning the sample into wells of a multi-well plate (optionally following a dilution step). Optionally, the sample may be partitioned into a plurality of compartments by partitioning the sample into wells of a microfluidic chip (optionally following a dilution step).

### Kits

The present invention also provides kits for the detection of nucleotide impurities in rAAV particles which are derived from AAV rep and/or cap genes. Accordingly, the present invention provides kits for use in a method of the invention.

A kit of the invention comprises means for indicating the presence of a nucleotide sequence corresponding to at least a portion of a rep gene and means for indicating the presence of a nucleotide sequence corresponding to at least a portion of a cap gene. Optionally, the kit comprises means for partitioning the sample into a plurality of compartments.

Optionally, the means for indicating the presence of a nucleotide sequence corresponding to at least a portion of a rep gene comprises means for producing an amplification product. Optionally, the means for indicating the presence of a nucleotide sequence corresponding to at least a portion of a cap gene comprises means for producing an amplification product.

Optionally the means for producing an amplification product is one or more nucleic acid primers, for example a forward PCR primer and a reverse PCR primer. Optionally, the means for indicating the presence of a nucleotide sequence corresponding to at least a portion of a rep gene is a forward and reverse primer capable of hybridising to an AAV rep gene. Optionally, the means for indicating the presence of a nucleotide sequence corresponding to at least a portion of a cap gene is a forward and reverse primer capable of hybridising to an AAV cap gene.

Optionally, the means for indicating the presence of a nucleotide sequence corresponding to at least a portion of a rep gene comprises means for producing a detectable signal. Optionally, the means for indicating the presence of a nucleotide sequence corresponding to at least a portion of a cap gene comprises means for producing a detectable signal. Detecting the respective detectable signals allows compartments that comprise i) a nucleotide sequence corresponding to the at least a portion of the rep gene but do not comprise a nucleotide sequence corresponding to at least a portion of the cap gene; ii) a nucleotide sequence corresponding to the at least a portion of the cap gene but do not comprise the nucleotide sequence corresponding to at least a portion of the rep gene; and iii) a nucleotide sequence corresponding to the at least a portion of the rep gene and a nucleotide sequence corresponding to the at least a portion of the cap gene to be detected.

In one embodiment, the present invention provides a kit comprising:
(a) forward and reverse PCR primers capable of hybridising to an AAV rep gene; and
(b) forward and reverse PCR primers capable of hybridising to an AAV cap gene.

Optionally, the forward and/or reverse PCR primers capable of hybridising to the AAV rep gene are primers as defined elsewhere herein. Optionally, the forward and/or reverse PCR primers capable of hybridising to the AAV cap gene are primers as defined elsewhere herein.

Optionally, the means for producing a detectable signal may be a nucleic acid probe. Thus, optionally, the means for indicating the presence of a nucleotide sequence corresponding to at least a portion of a rep gene comprises a nucleic acid probe for detecting an AAV rep gene capable of hybridising to a portion of the AAV rep gene. Optionally, the means for indicating the presence of a nucleotide sequence corresponding to at least a portion of a cap gene comprises a nucleic acid probe for detecting an AAV cap gene capable of hybridising to a portion of the AAV cap gene.

Thus, optionally, a kit of the invention may comprise nucleic acid probes for indicating the presence of at least a portion of a rep gene and/or at least a portion of a cap gene. Optionally, the kit may comprise a nucleic acid probe for detecting an AAV rep gene capable of hybridising to a portion of the AAV rep gene. Optionally, the kit may comprise a nucleic acid probe for detecting an AAV cap gene capable of hybridising to a portion of the AAV cap gene. Optionally, the nucleic acid probe for detecting the AAV rep gene is a nucleic acid probe as defined elsewhere herein. Optionally, the nucleic acid probe for detecting the AAV cap gene is a nucleic acid probe as defined elsewhere herein.

In one embodiment, a kit of the invention may comprise:
(a) forward and reverse PCR primers capable of hybridising to an AAV rep gene;
(b) forward and reverse PCR primers capable of hybridising to an AAV cap gene;
(c) a nucleic acid probe for detecting an AAV rep gene capable of hybridising to a portion of the AAV rep gene; and
(d) a nucleic acid probe for detecting an AAV cap gene capable of hybridising to a portion of the AAV cap gene.

Optionally, the means for indicating the presence of a nucleotide sequence corresponding to at least a portion of a rep gene and/or the means for indicating the presence of a nucleotide sequence corresponding to at least a portion of a cap gene may comprise a double-stranded DNA intercalating dye.

Thus, optionally, a kit of the invention may comprise a double-stranded DNA intercalating dye. Optionally, the double-stranded DNA intercalating dye is a fluorescent dye. Optionally, the double-stranded DNA intercalating dye is not significantly fluorescent when not bound to (intercalated with) double-stranded DNA. Optionally, the double-stranded DNA intercalating dye becomes fluorescent upon binding to (intercalation with) double-stranded DNA. Optionally, the double-stranded DNA intercalating dye is an EvaGreen® dye.

Optionally, a kit of the invention may comprise means for partitioning the sample into a plurality of compartments. Optionally, the kit may comprise droplet generator oil. Optionally, the kit may comprise a multi-well plate. Optionally, the kit may comprise a micro fluidic chip.

Optionally, a kit of the invention may comprise instructions for carrying out the method of the invention. These instructions may be present in the subject kits in a variety of forms, one or more of which may be present in the kit. One form in which these instructions may be present is as printed information on a suitable medium or substrate, e.g. a piece or pieces of paper on which the information is printed, in the packaging of the kit, in a package insert, etc. Yet another means would be a computer readable medium, e.g. diskette, CD, etc., on which the information has been recorded. Yet another means that may be present is a website address which may be used via the internet to access the information at a removed site. Any convenient means may be present in the kits.

### Pharmaceutical Compositions

A pharmaceutical composition comprising rAAV particles may be provided according to one aspect of the invention. The pharmaceutical composition may comprise a pharmaceutically acceptable excipient. The pharmaceutically acceptable excipients may comprise carriers, diluents and/or other medicinal agents, pharmaceutical agents or adjuvants, etc. Optionally, the pharmaceutically acceptable excipients comprise saline solution. Optionally, the pharmaceutically acceptable excipients comprise human serum albumin.

The pharmaceutical composition may be selected to comprise rAAV particles having a maximum desired level of rAAV particles which may be replication competent, rAAV particles comprising rep and cap sequences encapsidated in the same particle and/or comprising linked rep-cap sequences. Typically, pharmaceutical compositions comprising fewer, rather than more, such rAAV particles are preferred because it is highly desirable to minimise the number of AAV particles that may be pseudo-wild type upon administration of the rAAV particles for gene therapy in a subject.

The level of rAAV particles which may be pseudo-wild type, comprise rep and cap sequences encapsidated in the same particle and/or comprise linked rep-cap sequences may be determined by a method of the invention. For example, the pharmaceutical composition may comprise fewer than 1%, fewer than 0.1%, fewer than 0.01%, fewer than 0.001% or fewer than 0.0001% such rAAV, as determined by the methods of the invention.

### Purifying the Recombinant AAV

The methods may comprise a step of purifying the rAAV. In general, a step of purifying the rAAV will involve increasing the concentration of the rAAV compared to other components of a preparation. Optionally, the step of purifying the rAAV results in a concentrated rAAV preparation. Optionally, the step of purifying the rAAV results in an isolated rAAV.

Any suitable purification method may be used. Optionally, the step of purifying the rAAV is carried out using a technique selected from the group consisting of gradient density centrifugation (such as CsCl or lodixanol gradient density centrifugation), filtration, ion exchange chromatography, size exclusion chromatography, affinity chromatography and hydrophobic interaction chromatography.

Optionally, the method comprises further concentrating the rAAV using ultracentrifugation, tangential flow filtration, or gel filtration.

The method may further comprise a step of purifying the rAAV. Optionally, the step of purification occurs before a method of the invention. Optionally, the step of purification occurs after a method of the invention. Optionally, a method of the invention may be performed on a sample prior to a step of purification, and after a step of purification, and the level of rAAV particles which may be pseudo-wild type, comprise rep and cap sequences encapsidated in the same particle and/or comprise linked rep-cap sequences may be set by a method of the invention before and after the purification step may be compared.

The following numbered aspects represent particular embodiments of the invention.

1. A method of detecting adeno-associated virus (AAV) in a sample comprising recombinant adeno-associated virus (rAAV) particles which may be pseudo-wild type rAAV particles, said method comprising: a. obtaining the sample comprising rAAV particles; b. partitioning the sample into a plurality of compartments; c. performing assays for indicating the presence of a nucleotide sequence corresponding to at least a portion of a rep gene and the presence of a nucleotide sequence corresponding to at least a portion of a cap gene within each of the plurality of compartments; and d. detecting any compartments, within the plurality of compartments, that comprise, as indicated by the assays of step (c): i) a nucleotide sequence corresponding to the at least a portion of the rep gene but do not comprise a nucleotide sequence corresponding to the at least a portion of the cap gene; ii) a nucleotide sequence corresponding to the at least a portion of the cap gene but do not comprise the nucleotide sequence corresponding to the at least a portion of the rep gene; and iii) a nucleotide sequence corresponding to the at least a portion of the rep gene and a nucleotide sequence corresponding to the at least a portion of the cap gene; wherein the detection of compartments that comprise both a nucleotide sequence corresponding to the at least a portion of the rep gene and a nucleotide sequence corresponding to the at least a portion of the cap gene indicates that the sample comprises rAAV particles which may be pseudo-wild type rAAV particles.

2. The method of aspect 1, wherein step (d) comprises determining the number of compartments that comprise a nucleotide sequence corresponding to the at least a portion of the rep gene and a nucleotide sequence corresponding to the at least a portion of the cap gene, wherein the number of compartments that comprise both a nucleotide sequence corresponding to the at least a portion of the rep gene and a nucleotide sequence corresponding to the at least a portion of the cap gene indicates the quantity of rAAV in the sample which may be pseudo-wild type rAAV particles.

3. A method of quantifying adeno-associated virus (AAV) in a sample comprising recombinant adeno-associated virus (rAAV) particles which may be pseudo-wild type rAAV particles, said method comprising: a. obtaining the sample comprising rAAV particles; b. partitioning the sample into a plurality of compartments; c. performing assays for indicating the presence of a nucleotide sequence corresponding to at least a portion of a rep gene and the presence of a nucleotide sequence corresponding to at least a portion of a cap gene within each of the plurality of compartments; and d. determining the number of compartments, within the plurality of compartments, that comprise, as indicated by the assays of step (c): i) a nucleotide sequence corresponding to the at least a portion of the rep gene but not a nucleotide sequence corresponding to the at least a portion of the cap gene; ii) a nucleotide sequence corresponding to the at least a portion of the cap gene but not a nucleotide sequence corresponding to the at least a portion of the rep gene; and iii) a nucleotide sequence corresponding to the at least a portion of the rep gene and a nucleotide sequence corresponding to the at least a portion of the cap gene; wherein the number of compartments that comprise both a nucleotide sequence corresponding to the at least a portion of the rep gene and a nucleotide sequence corresponding to the at least a portion of the cap gene indicates the quantity of rAAV in the sample which may be pseudo-wild type rAAV particles.

4. The method of any one of aspects 1 to 3, wherein step (d) comprises detecting any compartments or determining the number of compartments, within the plurality of compartments, that do not comprise, as indicated by the assays of step (c), either a nucleotide sequence corresponding to a portion of the rep gene or a nucleotide sequence corresponding to a portion of the cap gene.

5. The method of any one of aspects 1 to 4, wherein the ratio of the number of compartments comprising a nucleotide sequence corresponding to the at least a portion of the rep gene and/or a nucleotide sequence corresponding to the at least a portion of the cap gene to the total number of compartments in the plurality of compartments detected is 1:4 or less, 1:5 or less, 1:6 or less, 1:7 or less, 1:8 or less, 1:9 or less, 1:10 or less, 1:15 or less, 1:20 or less, or 1:25 or less.

6. The method of any one of aspects 1 to 5, wherein the modal number of rAAV particles comprising a nucleotide sequence corresponding to the at least a portion of the rep gene and/or a nucleotide sequence corresponding to the at least a portion of the cap gene in each of the plurality of compartments is 0, preferably wherein at least 75%, at least 80%, at least 85%, at least 90%, or at least 95% of the plurality of compartments do not comprise an AAV particle comprising a nucleotide sequence corresponding to the at least a portion of the rep gene and/or a nucleotide sequence corresponding to the at least a portion of the cap gene.

7. The method of any one of aspects 1 to 6, wherein the modal non-zero number of rAAV particles comprising a nucleotide sequence corresponding to the at least a portion of the rep gene and/or a nucleotide sequence corresponding to the at least a portion of the cap gene in each of the plurality of compartments is 1.

8. The method of any one of aspects 1 to 7, wherein less than 5%, less than 2.5%, less than 2%, less than 1.5%, less than 1%, less than 0.5%, less than 0.4%, less than 0.3%, less than 0.2%, or less than 0.1% of the plurality of compartments comprise two or more AAV particles comprising a nucleotide sequence corresponding to the at least a portion of the rep gene and/or a nucleotide sequence corresponding to the at least a portion of the cap gene.

9. The method of any one of aspects 1 to 8, wherein the sample is diluted prior to step (b), and wherein steps (b)-(d) are performed on the diluted sample.

10. The method of any one of aspects 1 to 9, wherein the sample is diluted by two or more different dilution factors prior to step (b) to provide multiple diluted samples having different dilutions, and wherein steps (b)-(d) are performed on each of the multiple diluted samples.

11. The method of aspect 9 or 10, wherein if, following performing steps (b)-(d) on the diluted sample or each of the multiple diluted samples, the ratio of the number of compartments comprising a nucleotide sequence corresponding to the at least a portion of the rep gene and/or a nucleotide sequence corresponding to the at least a portion of the cap gene to the total number of compartments in the plurality of compartments detected is greater than 1:4, greater than 1:5, greater than 1:6, greater than 1:7, greater than 1:8, greater than 1:9, greater than 1:10, greater than 1:15, greater than 1:20, or greater than 1:25 the user identifies that the dilution factor is too low and the sample is diluted by a further greater dilution factor and steps (b)-(d) are performed on the further diluted sample, optionally wherein the step of diluting the sample by a further dilution factor is repeated one or more times if, following performing steps (b)-(d) on a further diluted sample, the ratio of the number of compartments comprising a nucleotide sequence corresponding to the at least a portion of the rep gene and/or a nucleotide sequence corresponding to the at least a portion of the cap gene to the total number of compartments in the plurality of compartments detected is greater than 1:4, greater than 1:5, greater than 1:6, greater than 1:7, greater than 1:8, greater than 1:9, greater than 1:10, greater than 1:15, greater than 1:20, or greater than 1:25.

12. The method of any one of aspects 1 to 11, wherein the number of compartments detected in step (d) comprises at least 5000, at least 6000, at least 7000, at least 8000, at least 9000, at least 10,000, at least 12,000, at least 14,000, at least 16,000, at least 18,000, or at least 20,000 compartments.

13. The method of any one of aspects 1 to 12, wherein the compartments are oil droplets.

14. The method of any one of aspects 1 to 13, wherein step (b) comprises emulsifying the sample with droplet generator oil.

15. The method of any one of aspects 1 to 14, wherein the assay for indicating the presence of a nucleotide sequence corresponding to at least a portion of the rep gene comprises a rep PCR assay, and/or wherein the assay for indicating the presence of a nucleotide sequence corresponding to at least a portion of the cap gene comprises a cap PCR assay.

16. The method of aspect 15, wherein the rep PCR assay utilises forward and reverse PCR primers which are capable of hybridising to at least a portion of an AAV rep gene.

17. The method of aspect 16, wherein the forward and/or reverse PCR primers are capable of hybridising to at least a portion of a sequence encoding a Rep78 and/or a Rep68 protein.

18. The method of aspect 16 or 17, wherein the forward and/or reverse PCR primers are capable of hybridising to at least a portion of a sequence corresponding to nucleotides 321-992 of the AAV2 genome set forth in SEQ ID NO: 1, or at least a portion of a sequence complementary thereto.

19. The method of any one of aspects 16 to 18 wherein the forward PCR primer is capable of hybridising to the reverse complement of at least a portion of a sequence corresponding to nucleotides 321-521 of the AAV2 genome set forth in SEQ ID NO: 1, and/or wherein the reverse PCR primer is capable of hybridising to at least a portion of a sequence corresponding to nucleotides 581-992 of the AAV2 genome set forth in SEQ ID NO: 1.

20. The method of any one of aspects 16 to 19, wherein the forward PCR primer: (i) is capable of hybridising to the reverse complement of at least a portion of a sequence corresponding to nucleotides 503-521 of the AAV2 genome set forth in SEQ ID NO: 1, optionally to the reverse complement of a sequence corresponding to nucleotides 503-521 of the AAV2 genome set forth in SEQ ID NO: 1; (ii) comprises or consists of the sequence [CGACTTTCTGACGGAATGG]; (iii) comprises or consists of a sequence at least 90%, preferably at least 95% identical to [CGACTTTCTGACGGAATGG]; or (iv) comprises or consists of a sequence that has between 1 and 3 substitutions compared to [CGACTTTCTGACGGAATGG]; and/or wherein the reverse PCR primer: (i) is capable of hybridising to at least a portion of a sequence corresponding to nucleotides 581-599 of the AAV2 genome set forth in SEQ ID NO: 1, optionally to a sequence corresponding to nucleotides 581-599 of the AAV2 genome set forth in SEQ ID NO: 1; (ii) comprises or consists of the sequence [CACGTGCATGTGGAAGTAG]; (iii) comprises or consists of a sequence at least 90%, preferably at least 95% identical to [CACGTGCATGTGGAAGTAG]; or (iv) comprises or consists of a sequence that has between 1 and 3 substitutions compared to [CACGTGCATGTGGAAGTAG].

21. The method of aspect 16 or 17, wherein the forward and/or reverse PCR primers are capable of hybridising to at least a portion of a sequence corresponding a gene encoding a Rep52, and/or Rep40 protein.

22. The method of any one of aspects 16, 17 or 21, wherein the forward and/or reverse PCR primers are capable of hybridising to at least a portion of a sequence corresponding to nucleotides 993-2252 of the AAV2 genome set forth in SEQ ID NO: 1, or at least a portion of a sequence complementary thereto.

23. The method of any one of aspects 16, 17, 21 or 22, wherein the forward PCR primer is capable of hybridising to the reverse complement of at least a portion of a sequence corresponding to nucleotides 993-1158 of the AAV2 genome set forth in SEQ ID NO: 1, and/or wherein the reverse PCR primer is capable of hybridising to at least a portion of a sequence corresponding to nucleotides 1226-2252 of the AAV2 genome set forth in SEQ ID NO: 1.

24. The method of any one of aspects 16, 17 or 21 to 23, wherein the forward PCR primer: (i) is capable of hybridising to the reverse complement of at least a portion of a sequence corresponding to nucleotides 1139-1158 of the AAV2 genome set forth in SEQ ID NO: 1, optionally to the reverse complement of a sequence corresponding to nucleotides 1139-1158 of the AAV2 genome set forth in SEQ ID NO: 1; (ii) comprises or consists of the sequence [TATGAGCCTGACTAAAACCG]; (iii) comprises or consists of a sequence at least 90%, preferably at least 95% identical to [TATGAGCCTGACTAAAACCG]; or (iv) comprises or consists of a sequence that has between 1 and 3 substitutions compared to [TATGAGCCTGACTAAAACCG]; and/or wherein the reverse PCR primer: (i) is capable of hybridising to at least a portion of a sequence corresponding to nucleotides 1226-1245 of the AAV2 genome set forth in SEQ ID NO: 1 optionally to a sequence corresponding to nucleotides 1226-1245 of the AAV2 genome set forth in SEQ ID NO: 1; (ii) comprises or consists of the sequence [GATCGTACCCGTTTAGTTCC]; (iii) comprises or consists of a sequence at least 90%, preferably at least 95% identical to [GATCGTACCCGTTTAGTTCC]; or (iv) comprises or consists of a sequence that has between 1 and 3 substitutions compared to [GATCGTACCCGTTTAGTTCC].

25. The method of any one of aspects 15-24, wherein the cap PCR assay utilises forward and reverse PCR primers which are capable of hybridising to at least a portion of an AAV cap gene.

26. The method of aspect 25, wherein the forward and/or reverse PCR primers are capable of hybridising to at least a portion a sequence encoding a VP1 capsid protein of an AAV capsid.

27. The method of aspect 25 or 26, wherein the forward and/or reverse PCR primers are capable of hybridising to at least a portion of a sequence encoding a VP3 capsid protein of an AAV capsid.

28. The method of any one of aspects 25 to 27, wherein the forward and/or reverse PCR primers are capable of hybridising to at least a portion of a sequence corresponding to nucleotides 2809-4407 of the AAV2 genome set forth in SEQ ID NO: 1, or at least a portion of a sequence complementary thereto.

29. The method of any one of aspects 25 to 28, wherein the forward PCR primer is capable of hybridising to the reverse complement of at least a portion of a sequence corresponding to nucleotides 2809-3247 of the AAV2 genome set forth in SEQ ID NO: 1, and/or wherein the reverse PCR primer is capable of hybridising to at least a portion of a sequence corresponding to nucleotides 3307-4407 of the AAV2 genome set forth in SEQ ID NO: 1.

30. The method of any one of aspects 25 to 29, wherein the forward PCR primer: (i) is capable of hybridising to the reverse complement of at least a portion of a sequence corresponding to nucleotides 3228-3247 of the AAV2 genome set forth in SEQ ID NO: 1, optionally to the reverse complement of a sequence corresponding to nucleotides 3228-3247 of the AAV2 genome set forth in SEQ ID NO: 1; (ii) comprises or consists of the sequence [GTTTACGGACTCGGAGTATC]; (iii) comprises or consists of a sequence at least 90%, preferably at least 95% identical to [GTTTACGGACTCGGAGTATC]; or (iv) comprises of consists of a sequence that has between 1 and 3 substitutions compared to [GTTTACGGACTCGGAGTATC]; and/or wherein the reverse PCR primer: (i) is capable of hybridising to at least a portion of a sequence corresponding to nucleotides 3307-3326 of the AAV2 genome set forth in SEQ ID NO: 1, optionally to a sequence corresponding to nucleotides 3307-3326 3228-3247 of the AAV2 genome set forth in SEQ ID NO: 1; (ii) comprises or consists of the sequence [TACTGAGGGACCATGAAGAC]; (iii) comprises or consists of a sequence at least 90%, preferably at least 95% identical to [TACTGAGGGACCATGAAGAC]; or (iv) comprises or consists of a sequence that has between 1 and 3 substitutions compared to [TACTGAGGGACCATGAAGAC], 31. The method of any one of aspects 15 to 30, wherein: (i) the forward PCR primer for the rep PCR assay binds to a sequence at least 50 bp, at least 55 bp, at least 60 bp, at least 65 bp, at least 70 bp, at least 75 bp, at least 80 bp, at least 85 bp, at least 90 bp, at least 100 bp, at least 100 bp, at least 120 bp, at least 130 bp, at least 140 bp, at least 150 bp, at least 200 bp or at least 250 bp in length, and/or less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp from the reverse complement of the sequence to which the reverse PCR primer for the rep PCR assay binds; and/or (ii) the forward PCR primer for the cap PCR assay binds to a sequence at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp from the reverse complement of the sequence to which the reverse PCR primer for the cap PCR assay binds.

32. The method of any one of aspects 15 to 31, wherein the amplification product produced by the rep PCR assay and/or cap PCR assay are at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or wherein the amplification products produced by the rep PCR assay and/or the cap PCR assay are less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp in length.

33. The method of any one of aspects 15 to 32, wherein an amplification product of the rep PCR assay is detected using a nucleic acid probe for detecting an AAV rep gene capable of hybridising to at least a portion of the AAV rep gene or the complement thereof, and/or wherein an amplification product of the cap PCR assay is detected using a nucleic acid probe for detecting a cap gene capable of hybridising to at least a portion of the cap gene or the complement thereof.

34. The method of aspect 33, wherein the nucleic acid probe for detecting a rep gene is a nucleic acid probe capable of hybridising to at least a portion of an AAV rep gene.

35. The method of aspect 33 or 34, wherein the nucleic acid probe for detecting a rep gene is a nucleic acid probe capable of hybridising to at least a portion of a sequence encoding a Rep78 and/or a Rep68 protein.

36. The method of any one of aspects 33 to 35, wherein the nucleic acid probe for detecting the rep gene is capable of hybridising to at least a portion of a sequence corresponding to positions 321-992 of the AAV2 genome set forth in SEQ ID NO: 1, or a sequence complementary thereto.

37. The method of any one of aspects 33 to 36, wherein the nucleic acid probe for detecting the rep gene: (i) is capable of hybridising to at least a portion of a sequence corresponding to nucleotides 522-580 of the AAV2 genome set forth in SEQ ID NO: 1, optionally to at least a portion of a sequence corresponding to nucleotides 525-544 of the AAV2 genome set forth in SEQ ID NO: 1, optionally to a sequence corresponding to nucleotides 525-544 of the AAV2 genome set forth in SEQ ID NO: 1; (ii) comprises or consists of the sequence [TCCGGGGCCTTACTCACACG]; (iii) comprises or consists of a sequence at least 90%, preferably at least 95% identical to [TCCGGGGCCTTACTCACACG]; (iv) comprises or consists of a sequence that has between 1 and 3 substitutions compared to [TCCGGGGCCTTACTCACACG]; or (v) comprises or consists of a sequence complementary to (i), (ii), (iii) or (iv).

38. The method of any one of aspects 33 to 35, wherein the nucleic acid probe for detecting a rep gene is a nucleic acid probe capable of hybridising to at least a portion of a sequence encoding a Rep52 and/or a Rep40 protein.

39. The method of any one of aspects 33 to 35 or 38, wherein the nucleic acid probe for detecting the rep gene is capable of hybridising to at least a portion of a sequence corresponding to positions 993-2252 of the AAV2 genome set forth in SEQ ID NO: 1, or a sequence complementary thereto.

40. The method of any one of aspects 33 to 35, 38 or 38, wherein the nucleic acid probe for detecting the rep gene: (i) is capable of hybridising to at least a portion of a sequence corresponding to nucleotides 1159-1225 of the AAV2 genome set forth in SEQ ID NO: 1, optionally to at least a portion of a sequence corresponding to nucleotides 1189-1210 of the AAV2 genome set forth in SEQ ID NO: 1, optionally to a sequence corresponding to nucleotides 1189-1210 of the AAV2 genome set forth in SEQ ID NO: 1; (ii) comprises or consists of the sequence [TGGAGGACATTTCCAGCAATCG]; (iii) comprises or consists of a sequence at least 90%, preferably at least 95% identical to [TGGAGGACATTTCCAGCAATCG]; (iv) comprises or consists of a sequence that has between 1 and 3 substitutions compared to [TGGAGGACATTTCCAGCAATCG]; or (v) comprises or consists of a sequence complementary to (i), (ii), (iii) or (iv).

41. The method of any one of aspects 33 to 40, wherein the nucleic acid probe for detecting a cap gene is a nucleic acid probe capable of hybridising to at least a portion of an AAV cap gene.

42. The method of any one of aspects 33 to 41, wherein the nucleic acid probe for detecting a cap gene is a nucleic acid probe capable of hybridising to at least a portion of a sequence encoding a VP3 capsid protein of an AAV capsid.

43. The method of any one of aspects 33 to 42, wherein the nucleic acid probe for detecting the cap gene is capable of hybridising to at least a portion of a sequence corresponding to positions 2809-4407 of the AAV2 genome set forth in SEQ ID NO: 1, or a sequence complementary thereto.

44. The method of any one of aspects 33 to 43, wherein the nucleic acid probe for detecting a cap gene: (i) is capable of hybridising to at least a portion of a sequence corresponding to nucleotides 3248-3306 of the AAV2 genome set forth in SEQ ID NO: 1, optionally to at least a portion of a sequence corresponding to nucleotides 3282-3300 of the AAV2 genome set forth in SEQ ID NO: 1, optionally to a sequence corresponding to nucleotides 3282-3300 of the AAV2 genome set forth in SEQ ID NO: 1; (ii) comprises or consists of the sequence [TGGAAACGGCGGGAGACAG]; (iii) comprises or consists of a sequence at least 90%, preferably at least 95% identical to [TGGAAACGGCGGGAGACAG]; (iv) comprises or consists of a sequence that has between 1 and 3 substitutions compared to [TGGAAACGGCGGGAGACAG]; or (v) comprises or consists of a sequence complementary to (i), (ii), (iii) or (iv).

45. The method of any one of aspects 33 to 44, wherein the nucleic acid probe for detecting the rep gene and/or the nucleic acid probe for detecting the cap gene comprise a detectable label.

46. The method of aspect 45, wherein the nucleic acid probe comprises the detectable labels at the 5′ end.

47. The method of aspect 45 or 46, wherein the detectable label is a fluorophore.

48. The method of aspect 47, wherein the nucleic acid probe for detecting the rep gene comprises a first fluorophore and/or the nucleic acid probe for detecting the cap gene comprises a second fluorophore. 49. The method of aspect 48, wherein one or both of the first or second fluorophores has an emission wavelength of around 530 nm to around 500 nm, around 520 nm to around 510 nm, around 515 nm or 517 nm and optionally is FAM, and/or wherein one of the first or second fluorophores has an emission wavelength of around 570 nm to around 530 nm, around 560 nm to around 540 nm, around 560 nm or 559 nm, and optionally is HEX.

50. The method of any one of aspects 45 to 49, wherein the nucleic acid probe comprises a quenching agent.

51. The method of aspect 50, wherein the nucleic acid probe comprises the quenching agent at the 3′ ends.

52. The method of aspect 50 or aspect 51, wherein the quenching agent is BHQ1.

53. The method of any one of aspects 46 to 51, wherein the nucleic acid probe comprises a fluorophore at its 5′ end and a quenching agent at its 3′ end, wherein upon binding of the probe to the amplification product of the PCR assay, the fluorophore is detached from the probe.

54. The method of aspect 52, wherein the nucleic acid probe is a TaqMan probe.

55. The method of any one of aspects 15 to 32, wherein the amplification product of the rep PCR assay and/or the amplification product of the cap PCR assay is/are detected using a double-stranded DNA intercalating dye.

56. The method of aspect 55, wherein the double-stranded DNA intercalating dye is a fluorescent dye.

57. The method of aspect 55 or 56, wherein the double-stranded DNA intercalating dye is an EvaGreen dye.

58. The method of any one of aspects 55 to 57, wherein the PCR primers capable of hybridising to the rep gene and the primers capable of hybridising to the cap gene have different Tₘ values.

59. The method of any one of aspects 55 to 58, wherein the amplification products of the PCR assays to indicate the presence of the rep and the cap gene are of different lengths.

60. The method of any one of aspects 1 to 59, wherein the rAAV particles are not, or the genomes of the rAAV particles are not, amplified prior to step (a).

61. The method of any one of aspects 1 to 60, wherein nucleic acid molecules comprised in the rAAV particles are not extracted prior to step (a) and/or (b), and/or wherein the rAAV particles are not disrupted prior to step (a) and/or (b).

62. The method of any one of aspects 1 to 61, wherein the sample comprising rAAV is obtained from an rcAAV assay.

63. The method of aspect 62, wherein the sample comprising rAAV is obtained from an rcAAV assay, wherein the method is a method for detecting AAV which may be replication competent pseudo-wild type rAAV particles, wherein the detection of compartments that comprise both a nucleotide sequence corresponding to the at least a portion of the rep gene and a nucleotide sequence corresponding to the at least a portion of the cap gene indicates that the sample comprises rAAV particles which may be pseudowild type rcAAV particles, optionally wherein step (d) comprises determining the number of compartments that comprise a nucleotide sequence corresponding to the at least a portion of the rep gene and a nucleotide sequence corresponding to the at least a portion of the cap gene, wherein the number of compartments that comprise both a nucleotide sequence corresponding to the at least a portion of the rep gene and a nucleotide sequence corresponding to the at least a portion of the cap gene indicates the quantity of rAAV in the sample which may be pseudo-wild type rcAAV particles.

64. The method of any one of aspects 1 to 61, further comprising performing an assay for indicating the presence of a nucleotide sequence corresponding to at least a portion of an inverted terminal repeat (ITR) within each of the plurality of compartments, optionally further comprising detecting any compartments, within the plurality of compartments, which comprise a nucleotide sequence corresponding to at least a portion of an ITR, wherein step (d) comprises determining the number of compartments, within the plurality of compartments, that comprise a nucleotide sequence corresponding to at least a portion of an ITR, optionally wherein step (d) comprises determining the number of compartments that comprise a nucleotide sequence corresponding to at least a portion of an ITR.

65. A kit comprising: (a) forward and reverse PCR primers capable of hybridising to an AAV rep gene; and (b) forward and reverse PCR primers capable of hybridising to an AAV cap gene.

66. The kit of aspect 64, wherein the forward and/or reverse PCR primers capable of hybridising to the AAV rep gene are forward and/or reverse PCR primers as defined in any one of aspects 17 to 23, and/or wherein the forward and/or reverse PCR primers capable of hybridising to the AAV cap gene are forward and/or reverse PCR primers as defined in any one of aspects 24 to 29.

67. The kit of aspect 65 or 66, further comprising: (c) a nucleic acid probe capable of hybridising to a portion of the AAV rep gene for detecting an AAV rep gene; and/or (d) a nucleic acid probe capable of hybridising to a portion of the AAV cap gene for detecting an AAV cap gene.

68. The kit of aspect 67, wherein the nucleic acid probe for detecting the AAV rep gene and/or the nucleic acid probe for detecting the AAV cap gene are nucleic acid probes as defined in any one of aspects 34 to 54.

69. The kit of aspect 65 or 66, further comprising a double-stranded DNA intercalating dye, optionally wherein the double-stranded DNA intercalating dye is a fluorescent dye, optionally wherein the fluorescent due is an EvaGreen dye.

70. The kit of any one of aspects 65 to 69, further comprising droplet generator oil.

71. The kit of any one of aspects 65 to 70 further comprising instructions for carrying out the method of any one of aspects 1 to 64.

72. A pharmaceutical composition comprising rAAV particles, wherein the proportion of AAV particles in the pharmaceutical composition which may be pseudo-wild type was determined by the method of any one of aspects 2 to 64.

73. The pharmaceutical composition of aspect 72, wherein the pharmaceutical composition comprises less than 1 in 10⁷, more preferably less than 1 in 10⁸ AAV which may be pseudo-wild type rAAV.

### EXAMPLES

The following Examples are presented in order to more fully illustrate the preferred embodiments of the disclosure. One of ordinary skill in the art will recognize the numerous modifications and variations that may be performed without altering the spirit or scope of the disclosure. Such modifications and variations are encompassed within the scope of the disclosure. These examples should in no way be construed as limiting the scope of the disclosed matter, as defined by the appended claims.

### Materials & Methods

### rAAV Samples

### Rep78Neg Helper Plasmid

To make the Rep78neg helper plasmid, nucleotides 200-4497 of wild type AAV2 (Genbank accession number AF043303; SEQ ID NO: 1) containing the rep and cap genes were cloned into the pUC19 vector (Yanisch-Perron et al (1985), Gene, 33:103-119). AAV2 nucleotides 4461-4497 were deleted to minimise sequence homology with the vector plasmid. To prevent Rep 78 expression while maintaining Rep 68 expression, the intron within the cloned rep gene was deleted. In order to provide for expression of Rep 52 following deletion of the intron, AAV2 nucleotides corresponding to rep 52 including the pl9 promoter were cloned immediately 3′ of the intron-less rep 68 gene. The majority of cap gene sequences were then deleted. Furthermore, the two p40 promoters (one in each of the Rep 68- and Rep 52-encoding rep gene duplications) were rendered non-functional by ablation of the TATA boxes (mutation of the T corresponding to AAV2 position 1823 and AAG corresponding to AAV2 positions 1826-1828 to C and CTC respectively). The resulting ‘rep cassette’ was then cloned into a plasmid comprising an 8342-nucleotide stretch comprising functional VA RNA I and IL, E2A and E4 genes from adenovirus (i.e. helper virus) serotype 5 (see SEQ ID NO: 4 of WO 2022/079429). The plasmid backbone, containing kanamycin resistance gene and bacterial origin of replication, was about 2.2 kb in length, resulting in a helper plasmid of 14021 nucleotides.

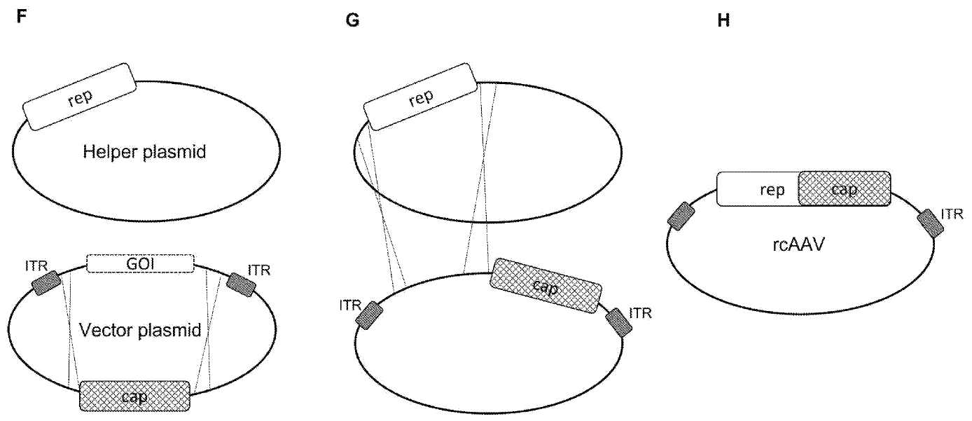

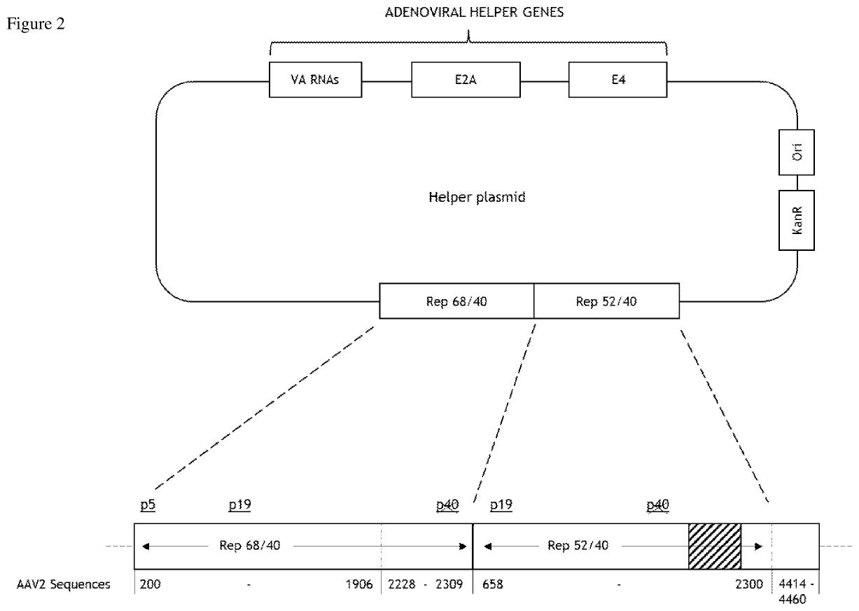

FIG. 2 is a schematic of the Rep78neg helper plasmid showing the main features. For the ‘rep cassette’ the portions of AAV2 sequence, by reference to the nucleotide positions of SEQ ID NO: 1, are indicated.

### Rep78Pos Helper Plasmid (Non-Optimised)

To make the Rep78pos helper plasmid (non-optimised), nucleotides 200-4460 of wild type AAV2 (Genbank accession number AF043303; SEQ ID NO: 1) containing the rep and cap genes were cloned into the pUC19 vector. The nucleotide sequence inadvertently contained a T in the place of a C at the position corresponding to position 1625 of AAV2, and a G in the place of an A at the position corresponding to position 1628 of AAV2. The encoded amino acid sequence was not changed by these differences. Next, AAV2 nucleotides 2330-4170 were deleted to remove a significant portion of the cap gene sequences. The ATG start codon in the rep gene driving Rep78/Rep68 expression was replaced with an ACG codon. The p40 promoter (contained within the rep gene) was rendered non-functional by ablation of the TATA box (mutation of AAG corresponding to AAV2 positions 1826-1828 to CTC). The resulting ‘rep cassette’ was then cloned into a plasmid comprising an 8342-nucleotide stretch comprising functional VA RNA I and II, E2A and E4 genes from adenovirus (i.e. helper virus) serotype 5 (see SEQ ID NO: 4 of WO 2022/079429). The plasmid backbone, containing kanamycin resistance gene and bacterial origin of replication, was about 2.2 kb in length, resulting in a helper plasmid of 12941 nucleotides.

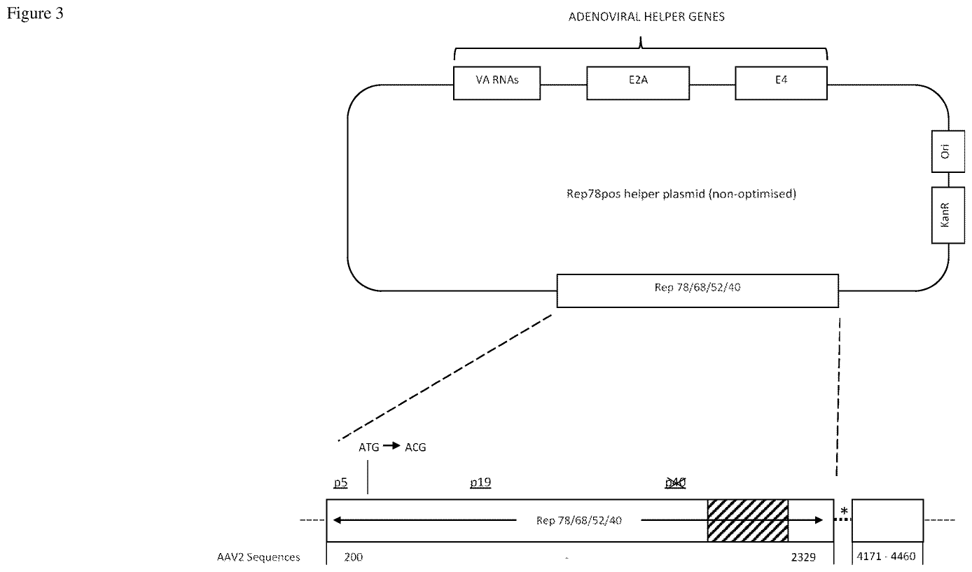

FIG. 3 is a schematic of the Rep78pos helper plasmid (non-optimised) showing the main features. For the ‘rep cassette’ the portions of AAV2 sequence, by reference to the nucleotide positions of SEQ ID NO: 1, are indicated.

### Rep78Pos Helper Plasmid (Optimised)

To make the Rep78pos helper plasmid (optimised), nucleotides 200-4460 of wild type AAV2 (Genbank accession number AF043303; SEQ ID NO: 1) containing the rep and cap genes were cloned into the pUC19 vector. Next, AAV2 nucleotides 2301-4410 were deleted to remove the majority of the cap gene sequences. The ATG start codon in the rep gene driving Rep78/Rep68 expression was replaced with an ACG codon. The p40 promoter (contained within the rep gene) was rendered non-functional by ablation of the TATA box (mutation of the T corresponding to AAV2 position 1823 and AAG corresponding to AAV2 positions 1826-1828 to C and CTC respectively). The resulting ‘rep cassette’ was then cloned into a plasmid comprising an 8342-nucleotide stretch comprising functional VA RNA I and II, E2A and E4 genes from adenovirus (i.e. helper virus) serotype 5 (see SEQ ID NO: 4 of WO 2022/079429). The plasmid backbone, containing kanamycin resistance gene and bacterial origin of replication, was about 2.2 kb in length, resulting in a helper plasmid of 12668 nucleotides.

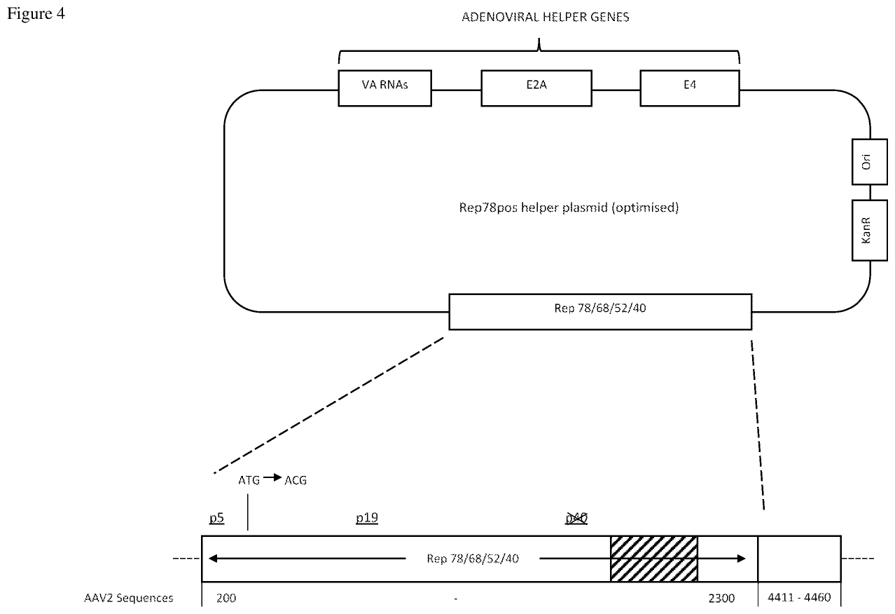

FIG. 4 is a schematic of the helper plasmid showing the main features. For the ‘rep cassette’ the portions of AAV2 sequence, by reference to the nucleotide positions of SEQ ID NO: 1, are indicated.

### Vector Plasmid (Split System)

To make the vector plasmid, nucleotides 200-4497 of wild type AAV2 (Genbank accession number AF043303; SEQ ID NO: 1) containing the rep and cap genes were cloned into the pUC19 vector. Two portions of rep gene sequence, between the p5 and pl9 promoters and between the pl9 and p40 promoters respectively, were then deleted to prevent Rep protein expression whilst maintaining the downstream cap gene under the regulation of the three native promoters. As for the helper plasmid described above, AAV2 nucleotides 4461-4497 were deleted to minimise sequence homology with the helper plasmid. To minimise or prevent translation of undesired products from potential initiation codons within the remaining promoter region, four ATG codons and one GTG codon were removed: ATGs at positions corresponding to AAV2 nucleotides 321-323, 955-957 and 993-995, and a GTG corresponding to AAV2 nucleotides 1014-1016, were deleted, whilst ATG at AAV2 nucleotides 766-768 was mutated to ATT. The AAV2 cap gene encoding the VPs 1, 2 and 3 immediately 3′ of the above promoter region was replaced with the corresponding sequence of an engineered cap gene, which cap gene is 3 nucleotides (equating to one additional encoded amino acid) longer than the AAV2 cap gene. The resulting ‘promoter-cap’ cassette was cloned into a plasmid backbone containing kanamycin resistance gene and bacterial origin of replication. Into this backbone was inserted an expression cassette, containing the transgene sequence linked to promoter and polyA transcription regulatory elements, flanked by AAV2 sequence comprising the native AAV2 ITRs (AAV2 nucleotides 1-145 and 4535-4679).

In the case of a vector plasmid comprising a 2672-nucleotide (ITR-to-ITR) Factor IX expression cassette, as used in Example 1, 2 and 4, the plasmid backbone was approximately 2.3 kb in length, resulting in an 8268-nucleotide vector plasmid. In the case of a vector plasmid comprising a 4713-nucleotide (ITR-to-ITR) Factor VIII expression cassette, as used in Example 3, the plasmid backbone was approximately 2.3 kb in length, resulting in a 10309-nucleotide vector plasmid.

In the case of a vector plasmid comprising a 2985-nucleotide (ITR-to-ITR) secreted embryonic alkaline phosphatase (SEAP) expression cassette, as used in Example 5, the engineered cap was replaced by the cap sequence of AAV9 resulting in a 8393-nucleotide vector plasmid P-468.

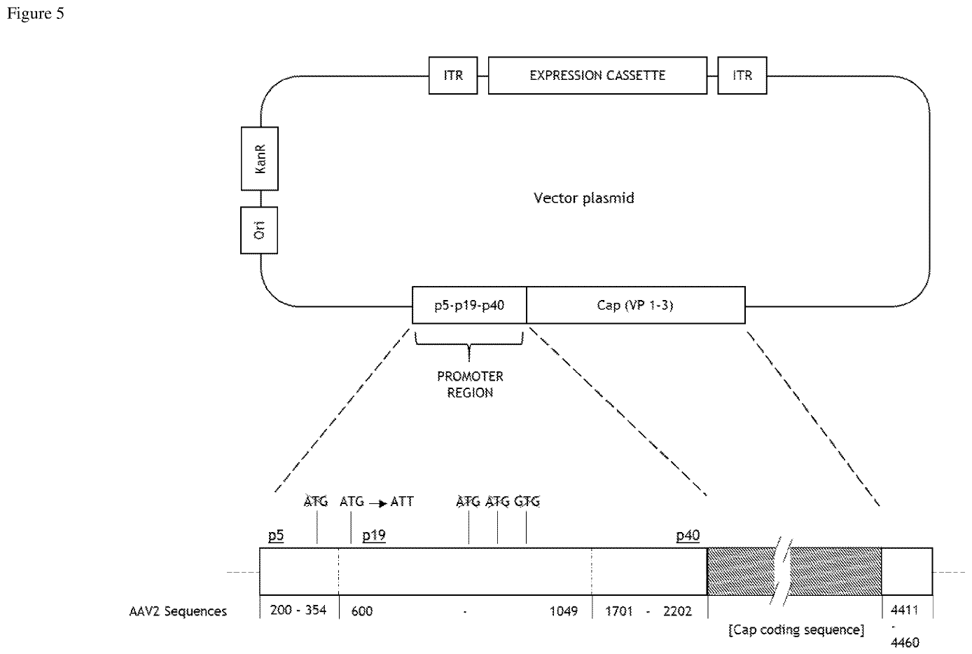

FIG. 5 is a schematic of the vector plasmid showing the main features. For the ‘promoter-cap’ cassette, the portions of AAV2 sequence, by reference to the nucleotide positions of SEQ ID NO: 1, are indicated.

### Non-Split Vector and Helper Plasmid

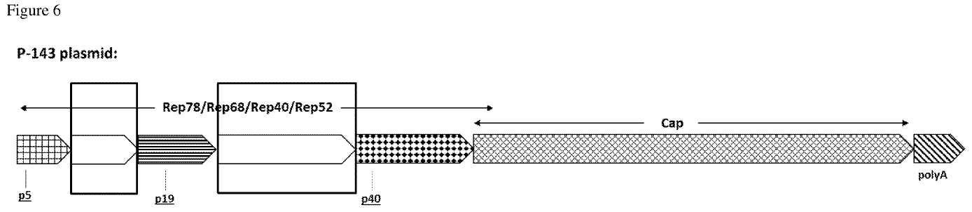

Two plasmids in a conventional “non-split” configuration (i.e. wherein the Rep and Cap functions are not split between two plasmids but retain their native AAV genomic arrangement on a single plasmid) were used for comparison. One plasmid (referred to herein as “P-144”) contained the same AdV helper and vector genome (Factor IX expression cassette) sequences as used in the trans-split two-plasmid system. The other plasmid (referred to herein as “P-143”) contained the same cap gene sequence as used in the trans-split two-plasmid system and an AAV2 rep cassette containing all four rep genes (FIG. 6). The ATG start codon in the rep gene driving Rep78/Rep68 expression was replaced with an ACG codon.

### Cell Cultivation, Transfection of HEK293T Cells and Preparation of Cell Lysates

HEK293T cells were maintained in adherent culture under standard conditions at 37° C., 95% relative humidity, and 5% v/v CO2 in Dulbecco's Modified Eagle's Medium (DMEM) supplemented with 10% Fetal Bovine Serum (FBS) and 1% GlutaMax™ (L-alanine-L-glutamine dipeptide). Cellular confluence during passaging ranged from 40-95%.

In example 1, HEK293T cells were transfected with two plasmids using different molar plasmid ratios for the trans-split two-plasmid system and for non-split system, while maintaining the total plasmid DNA amount: The helper:vector plasmid ratio for the transsplit two-plasmid system using the Rep78neg helper plasmid and the vector plasmid (comprising FIX expression cassette and engineered cap gene) was 1:3 and the AdV helper-vector genome expression cassette (P-144):cap-rep (P-143) plasmid ratio for nonsplit system was 1.8:1.

In examples 2 and 4, HEK293T cells were transfected with the vector plasmid comprising a FIX expression cassette and an engineered cap gene and the Rep78neg helper plasmid at a ratio of helper:vector plasmid of 1:3, the Rep78pos helper plasmid (non-optimised) at a ratio of helper:vector plasmid of 4:3, or the Rep78pos helper plasmid (optimised) at a ratio of helper:vector plasmid of 4:3. Per transfection, the total plasmid amount was the same for the Rep78neg two plasmid system, the Rep78pos (non-optimised) and the Rep78pos (optimised) two plasmid system.

In example 3, HEK293T cells were transfected with the vector plasmid comprising a FVIII expression cassette and an engineered cap gene and the Rep78neg helper plasmid at a ratio of helper:vector plasmid of 1:3, the Rep78pos helper plasmid (non-optimised) at a ratio of helper:vector plasmid of 4:3, or the Rep78pos helper plasmid (optimised) at a ratio of helper:vector plasmid of 4:3. Per transfection, the total plasmid amount was the same for the Rep78neg two plasmid system, the Rep78pos (non-optimised) and the Rep78pos (optimised) two plasmid system.

0.5×105 viable cells per cm2 culture area were seeded in 15-cm dishes in a volume of 22 ml DMEM, 10% FBS, 1% GlutaMax™ the day before transfection resulting in an average confluency of 60-80% on the day of transfection. PEI-DNA complexes were prepared in DMEM without supplements using the linear polyethylenimine transfection reagent PEIpro™ (Polyplus) according to the manufacturer's manual. An amount of 42 pg total plasmid DNA and a PEI-to-DNA ratio of 2:1 was maintained independent of the applied plasmid combination ratios. Cells were cultured until day 3 post-transfection, harvested in the medium and lysed by three freeze-thaw cycles (−80° C. and 37° C.). Cell debris was removed by centrifugation at 3,700×g for 30 min at 4° C. Lysates were treated with Denarase (Sartorius Stedim Biotech) to remove residual plasmid DNA and purified by affinity chromatography.

### Cell Cultivation, Transfection of HEK293 Cells in Ambrl5 and Preparation of Cell Lysates

HEK293 cells were maintained in suspension culture in shaker flasks at 120 rpm under standard conditions at 37° C., 70% relative humidity, and 5% v/v CO2 in BalanCD HEK293 media (FUJIFILM Irvine Scientific) supplemented with 4 mM L-glutamine (Thermo Fisher Scientific) with a splitting modus of 3 and 4 days.

In example 5, HEK293 cells were transfected with the vector plasmid comprising a SEAP expression cassette and an AAV9 cap gene and the Rep78neg helper plasmid at a ratio of helpervector plasmid of 1:3.

rAAV was produced in an Ambr® 15 Generation 2 Cell Culture 24 Bioreactor System (Sartorius Stedim Biotech GmbH). Ambr® 15 cc bioreactors with sparge were equilibrated with 12 mL of BalanCD HEK293 media (FUJIFILM Irvine Scientific) supplemented with 4 mM L-glutamine (Thermo Fisher Scientific) at 37° C., 40% DO and pH set-point 7.4. 20 pl EX-CELL® Antifoam (Merck) was added to each bioreactor every 12 hours starting directly post media fill. 1 molar sodium bicarbonate solution was used to adjust pH levels. HEK293 suspension cells were seeded into Ambr® 15 cc bioreactors at a final concentration of 2.5×106 viable cells per mL. Cells were transfected using the linear polyethylenimine transfection reagent PEIpro™ (Polyplus) according to the manufacturer's manual with a PEI-to-DNA ratio of 2:1. Per viable cell, a total plasmid DNA amount of 0.52 pg was applied. The PEI-DNA complexes were prepared in BalanCD HEK293 media supplemented with 4 mM L-glutamine. Cells were cultured until day 3 post-transfection, harvested in the medium and lysed by three freeze-thaw cycles (−80° C. and 37° C.). Cell debris was removed by centrifugation at 3,700×g for 30 min at 4° C. Lysates were treated with Denarase (c-LEcta GmbH) to remove residual plasmid DNA and purified by affinity chromatography.

ddPCR

Primers and probes were purchased from Metabion, Planegg (Germany). Probes were dual labelled with the fluorophore 6-carbofluorescein (6-FAM or “FAM” with an excitation maximum at 495 nm and an emission maximum at 520 nm) or hexachloro-fluorescein (“HEX” with an excitation maximum at 535 nm and an emission maximum at 556 nm) positioned at the 5′ end of the probe and the quencher (Black Hole Quencher 1 (BHQ-1 or “BHQ”) with an absorption range at 480-580 nm) positioned at the 3′ end.

TABLE 1
Primer and probe sequences

| SEQ ID | | |
|---|---|---|
| NO: | Name | Sequence |
| 2 | Q-008_rep1_fw | CGACTTTCTGACGGAA |
| | | TGG |
| 3 | Q-007_rep1_rv | CACGTGCATGTGGAAG |
| | | TAG |
| 4 | QP-048_rep1_FAM | FAM-TCCGGGGCCTTA |
| | | CTCACACG-BHQ1 |
| 5 | Q-010_cap_fw | GTTTACGGACTCGGAG |
| | | TATC |
| 6 | Q-009_cap_rv | TACTGAGGGACCATGA |
| | | AGAC |
| 7 | QP-049_cap_HEX | HEX-TGGAAACGGCGG |
| | | GAGACAG-BHQ1 |
| 8 | Q-150_rep2_fw | TATGAGCCTGACTAAA |
| | | ACCG |
| 9 | Q-151_rep2_rv | GATCGTACCCGTTTAG |
| | | TTCC |
| 10 | QP-050_rep2_FAM | FAM-TGGAGGACATTT |
| | | CCAGCAATCG-BHQ1 |
| 12 | Q-195_AAV9cap_fw | TCTTCACGGACTCAGA |
| | | CTATC |
| 13 | Q-196_AAV9cap_rev | TACTGAGGAATCATGA |
| | | AAACG |
| 14 | QP-077_AAV9cap_HEX | HEX-CAGCCCTCGTGA |
| | | GCCGAC-BHQ1 |

Forward primer Q-008 and reverse primer Q-007 are used to amplify a sequence in the rep68 gene, which is then detected with the FAM-labelled probe QP-048. This sequence is present in the helper plasmids of the trans-split two-plasmid system. Alternatively, primers Q-150 and Q-151 together with probe QP-050 can be used for detection of the rep gene sequence. Primer/probe set Q-010/Q-009/QP-049 binds in the cap sequence, such as an engineered cap sequence, which is present in the vector plasmid of the split two-plasmid system. Primer/probe set Q-195/Q-196/QP-077 binds in the AAV9 cap sequence which is present in the vector plasmid carrying the SEAP transgene of the split two-plasmid system.

Because the rep and cap sequences are initially on separate plasmids, observing a droplet positive for both FAM (detecting rep68 gene sequence) and HEX (detecting cap gene sequence) indicates that both rep and cap sequences are present in the same rAAV particle, either through co-linkage resulting from recombination between plasmids, or because rep and cap sequences have been co-packaged in the same rAAV particle, independent of the orientation of the arrangement of rep and cap sequences (see FIG. 7). Because a high dilution factor is applied to the rAAV particles for droplet generation, the likelihood of separate rAAV particles individually carrying rep and cap sequences being detected in the same droplet is low.

2-D (duplex) droplet digital PCR was carried out on the rAAV vector samples diluted in EB (10 mM Tris-Cl, pH 8.5) so that the sum of positive signals (signal for FAM, signal for HEX and dual signal for both FAM and HEX) was in the range of 500 to 4000 per 6 pL. The reaction mixtures were prepared using 10 pL ddPCR Supermix for Probes (no dUTP) and primers and probes at final concentrations of 0.9 pM and 0.25 pM, respectively. 6 pL sample dilution was added to the reaction mix, resulting in a final volume of 20 pL according to manufacturer's instructions. Primer and probe sequences are shown in Table 1. Samples were partitioned with droplet generator oil for probes (Bio-Rad) using a QX-200 droplet generator (Bio-Rad) according to manufacturer's instructions.

PCR amplification in the droplets was performed using a thermal cycler with the following parameters: 95° C. for 10 min, followed by 39 cycles of 94° C. for 30 s and 57° C. for 1 min followed by a final 98° C. heat treatment for 10 min. Droplet read-out of the PCR plate wells was carried out on the QX200 droplet reader (BIO-RAD) and data were analyzed with QuantaSoft or QX Manager software (BIO-RAD). Dilutions were adjusted so that the desired total number of all positive signals (signal for FAM, signal for HEX and dual signal for both FAM and HEX) were observed in around 20,000 droplets. A manual threshold was set over the negative droplets in both channels. For calculation of linked repcap molecules, the software-derived linkage concentration was used as described by Regan et al (PLoS ONE 10(3): eOl 18270). As a positive control, plasmid P-143, carrying both rep and cap sequences, was linearised with restriction enzyme Ndel and run in a parallel reaction to check primer performance and linkage. For all experiments described herein the linkage for the control plasmid was between 98.1%-100%. In example 5, plasmid P-496, carrying also the rep of AAV2 and a cap sequence from serotype AAV9, was linearised with restriction enzyme Xba1 to check primer performance and linkage.

### Example 1—Comparison of Split and Non-Split Samples

Detection of rep-cap sequences in the rAAV samples obtained from the non-split (P-143/P-144) or the trans-split (Rep78 negative helper plasmid together with a vector plasmid encoding a FIX transgene and engineered capsid) two-plasmid systems was carried out according to the method described above using primer/probe combinations Q-008/Q-007/F AM-labelled QP-048 for detection of the rep68 gene and Q-010/Q-009/HEX-labelled QP-049 for detection of the cap gene Specifically, one dilution was evaluated for the non-split and split sample. Samples were diluted to obtain less than 4000 positive droplets per reaction. Duplicate samples were measured.

### Results

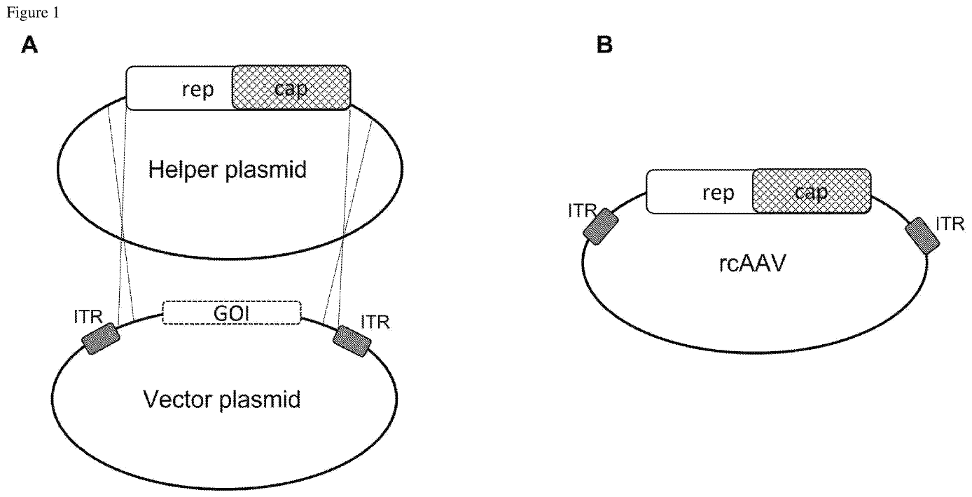

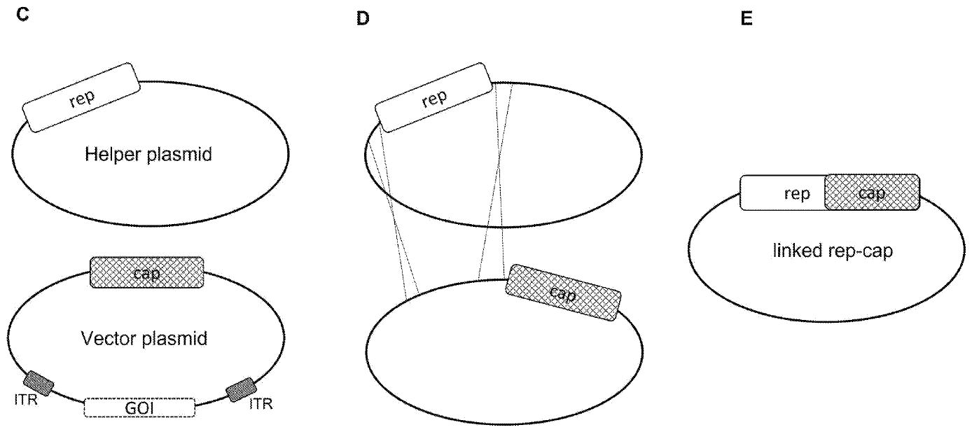

In the trans-split plasmid system, the rep-detecting primer/probe combination binds in the rep68 gene sequence which is only present in the helper plasmid whereas the cap-detecting primer/probe combination binds in the cap gene sequence which is present in the vector plasmid only. Contrary to that, in the non-split plasmid system rep and cap are present on the same helper plasmid (e.g. see P-143 plasmid). Detection of rep-cap species (i.e. rAAV which may be pseudo-wild type) is therefore expected to be higher in a non-split plasmid system, either through mis-packaging of rep-cap sequences present on the helper plasmid or arising from a single homologous recombination event, resulting in linked rep-cap species having recombined with the ITR sequences on the vector plasmid. Inverted terminal repeat (ITR) sequences provide the signal for packaging of the AAV vector genome into the capsid, and linked rep-cap sequences flanked by ITRs may form rAAV which may be replication competent (see FIGS. 1 A-B). On the other hand, in a split plasmid system, a recombination event is required to form linked rep-cap species (see FIG. 1C-E) and two recombination events are believed to be required to form linked repcap sequences flanked by ITRs (see FIG. 1F-H).

2-D (duplex) ddPCR analysis was performed to detect rAAV particles carrying both rep and cap genes. Observing a positive signal for both rep (FAM probe) and cap (HEX probe) indicates either the presence of AAV species with linked rep-cap sequences, or from rep and cap sequences independently (mis-)packaged in the same rAAV particle.

As shown in Table 2, rAAV samples generated using a trans-split plasmid system comprising a Rep78neg helper plasmid comprise significantly less rep-cap species than samples generated using a non-split plasmid system. Only packaging events leading to the configuration depicted in FIGS. 7 A to C are positive for both rep (FAM signal) and cap (HEX signal). ITRs may be present, but there is also the possibility of ITR independent packaging due to the presence of rep-binding site in the p5 promoter. Packaging of rep-cap sequences from the non-split plasmid system may at least partly be driven by this phenomenon. As shown in Table 2, in the non-split samples rep and cap are present in a 1:1 ratio. Because rep and cap are linked on the helper plasmid, it is most likely that rep and cap detected in the rAAV samples are also linked. In the trans-split samples, there is a significant excess of cap over rep. This indicates that at most the lower amount between the quantities of rep and cap sequences is an indicator of the maximum possible number of AAV species which contain rep and cap nucleic acid sequences, either co-packaged in the same rAAV particle or linked on the same DNA molecule. Therefore, the lower amount between the quantities of rep and cap sequences is an indicator of the maximum possible number of AAV which may be pseudo-wild type, because pseudo-wildtype AAV contains both rep and cap. Using duplex ddPCR for detection of rep and cap as described shows that the method has high sensitivity and that it can detect differences between different plasmid systems.

TABLE 2
rep-cap titers for rAA V samples obtained using split (Rep78neg
helper plasmid) and non-split expression systems

| | rel. cap | rel. rep | | % rep-cap | | |
|---|---|---|---|---|---|---|
| | [%] | [%] | ratio | of total | average | ratio |
| non-split | 0.34 | 0.34 | 1.0 | 0.0518 | 0.0523 | 34.45 |
| | 0.33 | 0.33 | 1.0 | 0.0528 | | |
| trans-split | 0.52 | 0.01179 | 44.2 | 0.0013 | 0.0015 | |
| | 0.50 | 0.01016 | 49.2 | 0.0018 | | |

rel.=relative

### Example 2—Comparison of Two Plasmid Systems Using Different Helper Plasmids and a FIX Containing Vector Plasmid

Detection of rep-cap sequences in the rAAV samples obtained from using Rep78neg helper plasmid, Rep78pos helper plasmid (non-optimised) and Rep78pos helper plasmid (optimised) together with a FIX transgene and capsid encoding vector plasmid in a split two-plasmid system was carried out according to the method described above using primer-probe combinations Q-008/Q-007/QP-048 for detection of the rep68 gene and Q-010/Q-009/QP-049 for detection of the cap gene.

Specifically, for the rAAV sample obtained with the Rep78neg helper plasmid, one dilution was evaluated in two replicates, whereby the number of all positives per reaction was around 800. For the rAAV sample obtained with the Rep78pos helper plasmid (nonoptimised), measurements were carried out in two dilutions in one or two replicates, keeping the number of all positives per reaction at around 1800 and around 900, respectively. For the rAAV sample obtained with the Rep78pos helper plasmid (nonoptimised), measurements were carried out in two dilutions in one or two replicates, keeping the number of all positives per reaction at around 1600 and around 800, respectively.

In a second step, the dilutions were optimised: for the rAAV sample obtained with the Rep78neg helper plasmid, two dilutions were evaluated in four replicates each, whereby the number of all positives per reaction were around 1700 and around 850, respectively. For rAAV samples obtained with the Rep78pos helper plasmid (non-optimised), measurements were carried out in two dilutions (three replicates each), keeping the number of all positives per reaction at around 1900 and around 950, respectively. For the rAAV samples obtained with the Rep78pos helper plasmid (non-optimised), measurements were carried out in two dilutions (three or four replicates each), keeping the number of all positives per reaction at around 1700 and around 850, respectively.

### Results

As shown in Table 3, rAAV samples generated using a trans-split two-plasmid system comprising a vector plasmid encoding FIX transgene and engineered cap gene together with a Rep78pos helper plasmid (optimised) showed about a five-fold reduction of rep-cap species over rAAV samples generated using a Rep78neg helper plasmid or a non-optimised Rep78pos helper plasmid. Both Rep78neg and non-optimised Rep78pos helper plasmid contain a fragment of the capsid gene. While these capsid gene fragments are small and do not encode any functional capsid, the sequences on these helper plasmids contain sequences which have homology to the engineered cap gene on the vector plasmid—without wishing to be bound by theory, this may be sufficient to drive homologous recombination to a small degree. While the number of rep-cap positive droplets for rep78neg helper plasmid samples is already very low compared to the non-split sample (see Example 1 and Table 2), optimising the helper plasmid can further decrease the number of rep-cap positive droplets. The disclosed method is therefore highly sensitive and can be used to detect rare events.

TABLE 3
rep-cap titers for r AAV samples obtained with vector plasmid
encoding engineered cap gene and FIX transgene and Rep78neg,
Rep78pos (non-optimised) or Rep78pos (optimised) helper plasmids
in a two-plasmid trans-split expression system

| | | | | rep-cap normalised |
|---|---|---|---|---|
| | rel. cap | rel. rep | % rep-cap | to Rep78neg helper |
| Sample | (%) | (%) | of total | plasmid sample |
| Rep78neg | 0.61 | 0.03 | 0.0072 | 100.00% |
| Rep78pos | 0.24 | 0.02 | 0.0055 | 76.67% |
| (non-optimised) | | | | |
| Rep78pos | 0.1 | 0.01 | 0.0012 | 16.42% |
| (optimised) | | | | |

Rel. = relative

### Example 3—Comparison of Two Plasmid Systems Using Different Helper Plasmids in the Context of a 4.71 kb AAV Vector Genome

Detection of rep-cap sequences in the rAAV samples obtained from using Rep78neg helper plasmid, Rep78pos helper plasmid (non-optimised) and Rep78pos helper plasmid (optimised) together with vector plasmid encoding a FVIII transgene with a total size of 4713 bp and engineered capsid in a trans-split two-plasmid system was carried out according to the method described above using primer probe combinations Q-008/Q-007/QP-048 for detection of the rep68 gene and Q-010/Q-009/QP-049 for detection of the cap gene.

Specifically, for the rAAV samples obtained with the Rep78neg helper plasmid, two dilutions were evaluated in two replicates each, whereby the number of all positives per reaction were around 1500 and around 750, respectively. For the rAAV samples obtained with the Rep78pos helper plasmid (non-optimised), measurements were carried out in two replicates each in two dilutions, keeping the number of all positives per reaction at around 1800 and around 900, respectively. For the rAAV samples obtained with the Rep78pos helper plasmid (non-optimised), measurements were carried out in two replicates each in two dilutions, keeping the number of all positives per reaction at around 1200 and around 600, respectively.

As shown in Table 4, rAAV samples comprising a FVIII transgene generated using the Rep78pos helper plasmid (optimised) in a trans-split two-plasmid system shows about a six-fold reduction of rep-cap species over samples generated using a Rep78neg helper plasmid or non-optimised Rep78pos helper plasmid. The disclosed method is therefore highly sensitive and can be used to detect rare events, irrespective of the transgene.

TABLE 4
rep-cap titers for rAAV samples obtained using Rep78neg, Rep78pos
(nonoptimised) or Rep78pos (optimised) helper plasmids and
vector plasmid encoding FVIII transgene with 4.71 kb and
engineered capsid in a trans-split two-plasmid system

| | | | | rep-cap normalised |
|---|---|---|---|---|
| | rel. cap | rel. rep | % rep-cap | to Rep78neg helper |
| Sample | (%) | (%) | of total | plasmid sample |
| Rep78neg | 1.02% | 0.05% | 0.0051 | 100.00% |
| Rep78pos | 0.23% | 0.04% | 0.0032 | 63.70% |
| (non-optimised) | | | | |
| Rep78pos | 0.10% | 0.02% | 0.0010 | 14.43% |
| (optimised) | | | | |

Rel. = relative

### Example 4—Alternative Primer/Probe Sets

In order to demonstrate the robustness of the method, alternative primers and probes for detection of the rep gene sequence were also tested.

Detection of rep-cap sequences in the rAAV samples obtained with the Rep78neg helper plasmid, Rep78pos helper plasmid (non-optimised) or Rep78pos helper plasmid (optimised) and a FIX transgene and capsid encoding vector plasmid in a trans-split two-plasmid system was performed according to the method described above using alternative primer-probe combinations Q-150/Q-151/QP-050 for detection of the rep gene and Q-010/Q-009/QP-049 for detection of the cap gene. Specifically, for the rAAV samples obtained with the Rep78neg helper plasmid, two dilutions were evaluated in two replicates each, whereby the number of all positives per reaction were around 1500 and around 750, respectively. For rAAV samples obtained with the Rep78pos helper plasmid (nonoptimised), measurements were carried out in one dilution (two replicates each), keeping the number of all positives per reaction at around 1000. For rAAV samples obtained with the Rep78pos helper plasmid (optimised), measurements were carried out in two dilutions (one or two replicates), keeping the number of all positives per reaction at around 1600 and around 800, respectively.

### Results

The duplex ddPCR performed with the alternative primer/probe pair (Q-150/Q-151/QP-050) for detection of the rep gene sequence led to similar results as the duplex ddPCR performed with primer/probe pair Q-008/Q-007/QP-048 and detecting a different rep gene sequence did not change the observed frequencies of rep-cap molecules detected in Example 2 (see Table 3), indicating that the disclosed method is robust and can detect rare events, regardless of the primer-probe combination used for detection.

TABLE 5
rep-cap titers for rAAV samples obtained using Rep78neg, Rep78pos
(nonoptimised) or Rep78pos (optimised) helper plasmids and
vector plasmid encoding FIX transgene and engineered capsid
in a trans-split two-plasmid expression system detected using
an alternative set of 5 ′primer/probe combination

| | | | | rep-cap |
|---|---|---|---|---|
| | | | | normalised to |
| | rel. cap | rel. rep | % rep-cap of | Rep78neg helper |
| Sample | (%) | (%) | total | plasmid sample |
| Rep78neg | 0.60 | 0.03 | 0.0036 | 100.00% |
| Rep78pos | 0.26 | 0.02 | 0.0028 | 77.17% |
| (non-optimised) | | | | |
| Rep78pos | 0.11 | 0.01 | 0.0006 | 17.73% |
| (optimised) | | | | |

### Example 5—Alternative rAAV Serotype

In order to demonstrate the adaptability of the method, alternative primers and probes for detection of the cap gene sequence for serotype 9 (AAV9) were also tested.

Detection of rep-cap sequences in the rAAV samples obtained with the Rep78neg helper plasmid and a SEAP transgene and capsid for serotype 9 encoding vector plasmid in a trans-split two-plasmid system was performed according to the method described above using alternative primer-probe combinations Q-008/Q-007/QP-048 for detection of the rep gene and Q-195/Q-196/QP-077 for detection of the cap gene. As control for primer/probe performance, detection of rep-cap sequences in a linearised rep-cap plasmid carrying sequences for rep serotype 2 and cap serotype 9 was also tested with the same primer/probe sets. Specifically, for the rAAV sample obtained with the Rep78neg helper plasmid (Rep78neg-AAV9), two experiments were performed. In the first experiment, three dilutions were evaluated in two replicates each, whereby the number of all positives per reaction were around 3600, around 1800 and around 900, respectively. In the second experiment, one dilution was evaluated in six replicates, whereby the number of all positive droplets per reaction were around 4000.

For the linearised rep-cap plasmid measurements were carried out in three dilution (two replicates each), keeping the number of all positives per reaction around 4000, around 2000 and around 1000, respectively.

### Results

In Table 6, the duplex ddPCR performed with the alternative primer/probe set for AAV9 serotype Q-008/Q-007/QP-048 for detection of the rep gene and Q-195/Q-196/QP-077 for detection of the cap gene for plasmid control (P-496_Rep2-cap9) show relative rep-cap titers of 98.5% confirming proper primer-probe performance and consistency of the method. Duplex ddPCR for rAAV sample Rep78neg-AAV9 carrying a SEAP transgene and a cap of serotype 9 detects linked relative rep-cap titers of 0.000125%.

The disclosed method is therefore adaptable and can be used to detect rare events, irrespective of the rAAV serotype.

TABLE 6
rep-cap titers for a linearised plasmid encoding rep of AA
V2 and cap of serotype 9 and a rAA V sample obtained using
Rep78neg helper plasmids and vector plasmid encoding SEAP
transgene andAAV9 capsid in a trans-split two-plasmid system

| Sample | rel. cap (%) | rel. rep (%) | % rep-cap of total |
|---|---|---|---|
| P-496 | 99.05 | 98.59 | 96.8477 |
| Rep78neg-AAV9 | 0.66 | 0.06 | 0.0025 |

1-24. (canceled)

25. A method of detecting adeno-associated virus (AAV) in a sample comprising recombinant adeno-associated virus (rAAV) particles which may be pseudo-wild type rAAV particles, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. detecting any compartments, within the plurality of compartments, that comprise, as indicated by the assays of step (c):
i) a nucleotide sequence corresponding to the at least a portion of the rep gene but do not comprise a nucleotide sequence corresponding to the at least a portion of the cap gene;
ii) a nucleotide sequence corresponding to the at least a portion of the cap gene but do not comprise the nucleotide sequence corresponding to the at least a portion of the rep gene; and
iii) a nucleotide sequence corresponding to the at least a portion of the rep gene and a nucleotide sequence corresponding to the at least a portion of the cap gene;
wherein the detection of compartments that comprise both a nucleotide sequence corresponding to the at least a portion of the rep gene and a nucleotide sequence corresponding to the at least a portion of the cap gene indicates that the sample comprises rAAV particles which may be pseudo-wild type rAAV particles, and wherein step (d) comprises determining the number of compartments that comprise a nucleotide sequence corresponding to the at least a portion of the rep gene and a nucleotide sequence corresponding to the at least a portion of the cap gene, wherein the number of compartments that comprise both a nucleotide sequence corresponding to the at least a portion of the rep gene and a nucleotide sequence corresponding to the at least a portion of the cap gene indicates the quantity of rAAV in the sample which may be pseudo-wild type rAAV particles.

26. A method of quantifying adeno-associated virus (AAV) in a sample comprising recombinant adeno-associated virus (rAAV) particles which may be pseudo-wild type rAAV particles, said method comprising:
a. obtaining the sample comprising rAAV particles;
b. partitioning the sample into a plurality of compartments;
c. performing assays for indicating the presence of a nucleotide sequence corresponding to at least a portion of a rep gene and the presence of a nucleotide sequence corresponding to at least a portion of a cap gene within each of the plurality of compartments; and
d. determining the number of compartments, within the plurality of compartments, that comprise, as indicated by the assays of step (c):
i) a nucleotide sequence corresponding to the at least a portion of the rep gene but not a nucleotide sequence corresponding to the at least a portion of the cap gene;
ii) a nucleotide sequence corresponding to the at least a portion of the cap gene but not a nucleotide sequence corresponding to the at least a portion of the rep gene; and
iii) a nucleotide sequence corresponding to the at least a portion of the rep gene and a nucleotide sequence corresponding to the at least a portion of the cap gene;
wherein the number of compartments that comprise both a nucleotide sequence corresponding to the at least a portion of the rep gene and a nucleotide sequence corresponding to the at least a portion of the cap gene indicates the quantity of rAAV in the sample which may be pseudo-wild type rAAV particles.
wherein step (d) comprises detecting any compartments or determining the number of compartments, within the plurality of compartments, that do not comprise, as indicated by the assays of step (c), either a nucleotide sequence corresponding to a portion of the rep gene or a nucleotide sequence corresponding to a portion of the cap gene.

27. The method of claim 26, wherein:
(i) the ratio of the number of compartments comprising a nucleotide sequence corresponding to the at least a portion of the rep gene and/or a nucleotide sequence corresponding to the at least a portion of the cap gene to the total number of compartments in the plurality of compartments detected is 1:4 or less, 1:5 or less, 1:6 or less, 1:7 or less, 1:8 or less, 1:9 or less, 1:10 or less, 1:15 or less, 1:20 or less, or 1:25 or less;
(ii) a modal number of rAAV particles comprising a nucleotide sequence corresponding to the at least a portion of the rep gene and/or a nucleotide sequence corresponding to the at least a portion of the cap gene in each of the plurality of compartments is 0, preferably wherein at least 75%, at least 80%, at least 85%, at least 90%, or at least 95% of the plurality of compartments do not comprise an AAV particle comprising a nucleotide sequence corresponding to the at least a portion of the rep gene and/or a nucleotide sequence corresponding to the at least a portion of the cap gene;
(iii) a modal non-zero number of rAAV particles comprising a nucleotide sequence corresponding to the at least a portion of the rep gene and/or a nucleotide sequence corresponding to the at least a portion of the cap gene in each of the plurality of compartments is 1; and/or
(iv) less than 5%, less than 2.5%, less than 2%, less than 1.5%, less than 1%, less than 0.5%, less than 0.4%, less than 0.3%, less than 0.2%, or less than 0.1% of the plurality of compartments comprise two or more AAV particles comprising a nucleotide sequence corresponding to the at least a portion of the rep gene and/or a nucleotide sequence corresponding to the at least a portion of the cap gene.

28. The method of claim 25, wherein:
(i) the sample is diluted prior to step (b), and wherein steps (b)-(d) are performed on the diluted sample; and/or
(ii) the sample is diluted by two or more different dilution factors prior to step (b) to provide multiple diluted samples having different dilutions, and wherein steps (b)-(d) are performed on each of the multiple diluted samples;
optionally wherein if, following performing steps (b)-(d) on the diluted sample or each of the multiple diluted samples, the ratio of the number of compartments comprising a nucleotide sequence corresponding to the at least a portion of the rep gene and/or a nucleotide sequence corresponding to the at least a portion of the cap gene to the total number of compartments in the plurality of compartments detected is greater than 1:4, greater than 1:5, greater than 1:6, greater than 1:7, greater than 1:8, greater than 1:9, greater than 1:10, greater than 1:15, greater than 1:20, or greater than 1:25 the user identifies that the dilution factor is too low and the sample is diluted by a further greater dilution factor and steps (b)-(d) are performed on the further diluted sample, optionally wherein the step of diluting the sample by a further dilution factor is repeated one or more times if, following performing steps (b)-(d) on a further diluted sample, the ratio of the number of compartments comprising a nucleotide sequence corresponding to the at least a portion of the rep gene and/or a nucleotide sequence corresponding to the at least a portion of the cap gene to the total number of compartments in the plurality of compartments detected is greater than 1:4, greater than 1:5, greater than 1:6, greater than 1:7, greater than 1:8, greater than 1:9, greater than 1:10, greater than 1:15, greater than 1:20, or greater than 1:25, wherein the number of compartments detected in step (d) comprises at least 5000, at least 6000, at least 7000, at least 8000, at least 9000, at least 10,000, at least 12,000, at least 14,000, at least 16,000, at least 18,000, or at least 20,000 compartments and wherein the compartments are oil droplets, and/or wherein step (b) comprises emulsifying the sample with droplet generator oil.

29. The method of claim 25, wherein the assay for indicating the presence of a nucleotide sequence corresponding to at least a portion of the rep gene comprises a rep PCR assay, and/or wherein the assay for indicating the presence of a nucleotide sequence corresponding to at least a portion of the cap gene comprises a cap PCR assay, optionally wherein:
(A) the rep PCR assay utilises forward and reverse PCR primers which are capable of hybridising to at least a portion of an AAV rep gene, optionally wherein:
(1) the forward and/or reverse PCR primers are capable of hybridising to at least a portion of a sequence encoding a Rep78 and/or a Rep68 protein; optionally wherein:
(i) the forward and/or reverse PCR primers are capable of hybridising to at least a portion of a sequence corresponding to nucleotides 321-992 of the AAV2 genome set forth in SEQ ID NO: 1, or at least a portion of a sequence complementary thereto; and/or
(ii) the forward PCR primer is capable of hybridising to the reverse complement of at least a portion of a sequence corresponding to nucleotides 321-521 of the AAV2 genome set forth in SEQ ID NO: 1, and/or wherein the reverse PCR primer is capable of hybridising to at least a portion of a sequence corresponding to nucleotides 581-992 of the AAV2 genome set forth in SEQ ID NO: 1; and/or
(iii) the forward PCR primer:
(i) is capable of hybridising to the reverse complement of at least a portion of a sequence corresponding to nucleotides 503-521 of the AAV2 genome set forth in SEQ ID NO: 1, optionally to the reverse complement of a sequence corresponding to nucleotides 503-521 of the AAV2 genome set forth in SEQ ID NO: 1;
(ii) comprises or consists of the sequence [CGACTTTCTGACGGAATGG];
(iii) comprises or consists of a sequence at least 90%, preferably at least 95% identical to [CGACTTTCTGACGGAATGG]; or
(iv) comprises or consists of a sequence that has between 1 and 3 substitutions compared to [CGACTTTCTGACGGAATGG];
and/or wherein the reverse PCR primer:
(i) is capable of hybridising to at least a portion of a sequence corresponding to nucleotides 581-599 of the AAV2 genome set forth in SEQ ID NO: 1, optionally to a sequence corresponding to nucleotides 581-599 of the AAV2 genome set forth in SEQ ID NO: 1;
(ii) comprises or consists of the sequence [CACGTGCATGTGGAAGTAG];
(iii) comprises or consists of a sequence at least 90%, preferably at least 95% identical to [CACGTGCATGTGGAAGTAG]; or
(iv) comprises or consists of a sequence that has between 1 and 3 substitutions compared to [CACGTGCATGTGGAAGTAG].
and/or
(2) the forward and/or reverse PCR primers are capable of hybridising to at least a portion of a sequence corresponding a gene encoding a Rep52, and/or Rep40 protein; optionally wherein
(i) the forward and/or reverse PCR primers are capable of hybridising to at least a portion of a sequence corresponding to nucleotides 993-2252 of the AAV2 genome set forth in SEQ ID NO: 1, or at least a portion of a sequence complementary thereto; and/or
(ii) the forward PCR primer is capable of hybridising to the reverse complement of at least a portion of a sequence corresponding to nucleotides 993-1158 of the AAV2 genome set forth in SEQ ID NO: 1, and/or wherein the reverse PCR primer is capable of hybridising to at least a portion of a sequence corresponding to nucleotides 1226-2252 of the AAV2 genome set forth in SEQ ID NO: 1; and/or
(iii) the forward PCR primer:
(i) is capable of hybridising to the reverse complement of at least a portion of a sequence corresponding to nucleotides 1139-1158 of the AAV2 genome set forth in SEQ ID NO: 1, optionally to the reverse complement of a sequence corresponding to nucleotides 1139-1158 of the AAV2 genome set forth in SEQ ID NO: 1;
(ii) comprises or consists of the sequence [TATGAGCCTGACTAAAACCG];
(iii) comprises or consists of a sequence at least 90%, preferably at least 95% identical to [TATGAGCCTGACTAAAACCG]; or
(iv) comprises or consists of a sequence that has between 1 and 3 substitutions compared to [TATGAGCCTGACTAAAACCG];
and/or wherein the reverse PCR primer:
(i) is capable of hybridising to at least a portion of a sequence corresponding to nucleotides 1226-1245 of the AAV2 genome set forth in SEQ ID NO: 1 optionally to a sequence corresponding to nucleotides 1226-1245 of the AAV2 genome set forth in SEQ ID NO: 1;
(ii) comprises or consists of the sequence [GATCGTACCCGTTTAGTTCC];
(iii) comprises or consists of a sequence at least 90%, preferably at least 95% identical to [GATCGTACCCGTTTAGTTCC]; or
(iv) comprises or consists of a sequence that has between 1 and 3 substitutions compared to [GATCGTACCCGTTTAGTTCC]
and/or
(B) the cap PCR assay utilises forward and reverse PCR primers which are capable of hybridising to at least a portion of an AAV cap gene, optionally wherein:
(1) the forward and/or reverse PCR primers are capable of hybridising to at least a portion a sequence encoding a VP1 capsid protein of an AAV capsid; and/or
(2) the forward and/or reverse PCR primers are capable of hybridising to at least a portion of a sequence encoding a VP3 capsid protein of an AAV capsid, optionally wherein
(i) the forward and/or reverse PCR primers are capable of hybridising to at least a portion of a sequence corresponding to nucleotides 2809-4407 of the AAV2 genome set forth in SEQ ID NO: 1, or at least a portion of a sequence complementary thereto; and/or
(ii) the forward PCR primer is capable of hybridising to the reverse complement of at least a portion of a sequence corresponding to nucleotides 2809-3247 of the AAV2 genome set forth in SEQ ID NO: 1, and/or wherein the reverse PCR primer is capable of hybridising to at least a portion of a sequence corresponding to nucleotides 3307-4407 of the AAV2 genome set forth in SEQ ID NO: 1; and/or
(iii) the forward PCR primer:
(i) is capable of hybridising to the reverse complement of at least a portion of a sequence corresponding to nucleotides 3228-3247 of the AAV2 genome set forth in SEQ ID NO: 1, optionally to the reverse complement of a sequence corresponding to nucleotides 3228-3247 of the AAV2 genome set forth in SEQ ID NO: 1;
(ii) comprises or consists of the sequence [GTTTACGGACTCGGAGTATC];
(iii) comprises or consists of a sequence at least 90%, preferably at least 95% identical to [GTTTACGGACTCGGAGTATC]; or
(iv) comprises of consists of a sequence that has between 1 and 3 substitutions compared to [GTTTACGGACTCGGAGTATC];
and/or wherein the reverse PCR primer:
(i) is capable of hybridising to at least a portion of a sequence corresponding to nucleotides 3307-3326 of the AAV2 genome set forth in SEQ ID NO: 1, optionally to a sequence corresponding to nucleotides 3307-3326 3228-3247 of the AAV2 genome set forth in SEQ ID NO: 1;
(ii) comprises or consists of the sequence [TACTGAGGGACCATGAAGAC];
(iii) comprises or consists of a sequence at least 90%, preferably at least 95% identical to [TACTGAGGGACCATGAAGAC]; or
(iv) comprises or consists of a sequence that has between 1 and 3 substitutions compared to [TACTGAGGGACCATGAAGAC].

30. The method of claim 29, wherein:
(i) the forward PCR primer for the rep PCR assay binds to a sequence at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp from the reverse complement of the sequence to which the reverse PCR primer for the rep PCR assay binds; and/or
(ii) the forward PCR primer for the cap PCR assay binds to a sequence at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp from the reverse complement of the sequence to which the reverse PCR primer for the cap PCR assay binds; and/or
(iii) the amplification product produced by the rep PCR assay and/or cap PCR assay are at least 50 bp, at least 55 bp, at least 60 bp, at least 65 bp, at least 70 bp, at least 75 bp, at least 80 bp, at least 85 bp, at least 90 bp, at least 100 bp, at least 110 bp, at least 120 bp, at least 130 bp, at least 140 bp, at least 150 bp, at least 200 bp or at least 250 bp in length, and/or wherein the amplification products produced by the rep PCR assay and/or the cap PCR assay are less than 1.8 kb, less than 1.6 kb, less than 1.5 kb, less than 1.4 kb, less than 1.3 kb, less than 1.2 kb, less than 1.1 kb, less than 1.0 kb, less than 900 bp, less than 800 bp, less than 700 bp, less than 600 bp, less than 500 bp, less than 400 bp or less than 300 bp in length.

31. The method of claim 25, wherein an amplification product of the rep PCR assay is detected using a nucleic acid probe for detecting an AAV rep gene capable of hybridising to at least a portion of the AAV rep gene or the complement thereof, and/or wherein an amplification product of the cap PCR assay is detected using a nucleic acid probe for detecting a cap gene capable of hybridising to at least a portion of the cap gene or the complement thereof, optionally wherein:
(A) the nucleic acid probe for detecting a rep gene is a nucleic acid probe capable of hybridising to at least a portion of an AAV rep gene, optionally wherein:
(a) the nucleic acid probe for detecting a rep gene is a nucleic acid probe capable of hybridising to at least a portion of a sequence encoding a Rep78 and/or a Rep68 protein; optionally wherein:
(i) the nucleic acid probe for detecting the rep gene is capable of hybridising to at least a portion of a sequence corresponding to positions 321-992 of the AAV2 genome set forth in SEQ ID NO: 1, or a sequence complementary thereto; and/or
(ii) the nucleic acid probe for detecting the rep gene:
(i) is capable of hybridising to at least a portion of a sequence corresponding to nucleotides 522-580 of the AAV2 genome set forth in SEQ ID NO: 1, optionally to at least a portion of a sequence corresponding to nucleotides 525-544 of the AAV2 genome set forth in SEQ ID NO: 1, optionally to a sequence corresponding to nucleotides 525-544 of the AAV2 genome set forth in SEQ ID NO: 1;
(ii) comprises or consists of the sequence [TCCGGGGCCTTACTCACACG];
(iii) comprises or consists of a sequence at least 90%, preferably at least 95% identical to [TCCGGGGCCTTACTCACACG];
(iv) comprises or consists of a sequence that has between 1 and 3 substitutions compared to [TCCGGGGCCTTACTCACACG];
or
(v) comprises or consists of a sequence complementary to (i), (ii), (iii) or (iv);
and/or
(b) the nucleic acid probe for detecting a rep gene is a nucleic acid probe capable of hybridising to at least a portion of a sequence encoding a Rep52 and/or a Rep40 protein; optionally wherein:
(i) the nucleic acid probe for detecting the rep gene is capable of hybridising to at least a portion of a sequence corresponding to positions 993-2252 of the AAV2 genome set forth in SEQ ID NO: 1, or a sequence complementary thereto; and/or
(ii) the nucleic acid probe for detecting the rep gene:
(i) is capable of hybridising to at least a portion of a sequence corresponding to nucleotides 1159-1225 of the AAV2 genome set forth in SEQ ID NO: 1, optionally to at least a portion of a sequence corresponding to nucleotides 1189-1210 of the AAV2 genome set forth in SEQ ID NO: 1, optionally to a sequence corresponding to nucleotides 1189-1210 of the AAV2 genome set forth in SEQ ID NO: 1;
(ii) comprises or consists of the sequence [TGGAGGACATTTCCAGCAATCG];
(iii) comprises or consists of a sequence at least 90%, preferably at least 95% identical to [TGGAGGACATTTCCAGCAATCG];
(iv) comprises or consists of a sequence that has between 1 and 3 substitutions compared to [TGGAGGACATTTCCAGCAATCG]; or
(v) comprises or consists of a sequence complementary to (i), (ii), (iii) or (iv); and/or
(B) the nucleic acid probe for detecting a cap gene is a nucleic acid probe capable of hybridising to at least a portion of an AAV cap gene; optionally wherein:
(a) the nucleic acid probe for detecting a cap gene is a nucleic acid probe capable of hybridising to at least a portion of a sequence encoding a VP3 capsid protein of an AAV capsid; optionally wherein:
(i) the nucleic acid probe for detecting the cap gene is capable of hybridising to at least a portion of a sequence corresponding to positions 2809-4407 of the AAV2 genome set forth in SEQ ID NO: 1, or a sequence complementary thereto; and/or
(ii) the nucleic acid probe for detecting a cap gene:
(i) is capable of hybridising to at least a portion of a sequence corresponding to nucleotides 3248-3306 of the AAV2 genome set forth in SEQ ID NO: 1, optionally to at least a portion of a sequence corresponding to nucleotides 3282-3300 of the AAV2 genome set forth in SEQ ID NO: 1, optionally to a sequence corresponding to nucleotides 3282-3300 of the AAV2 genome set forth in SEQ ID NO: 1;
(ii) comprises or consists of the sequence [TGGAAACGGCGGGAGACAG];
(iii) comprises or consists of a sequence at least 90%, preferably at least 95% identical to [TGGAAACGGCGGGAGACAG];
(iv) comprises or consists of a sequence that has between 1 and 3 substitutions compared to [TGGAAACGGCGGGAGACAG];
or
(v) comprises or consists of a sequence complementary to (i), (ii), (iii) or (iv).

32. The method of claim 25, wherein the nucleic acid probe for detecting the rep gene and/or the nucleic acid probe for detecting the cap gene comprise a detectable label, optionally wherein:
(i) the nucleic acid probe comprises the detectable labels at the 5′ end, optionally wherein the detectable label is a fluorophore; and/or
(ii) the nucleic acid probe for detecting the rep gene comprises a first fluorophore and/or the nucleic acid probe for detecting the cap gene comprises a second fluorophore, optionally wherein one or both of the first or second fluorophores has an emission wavelength of around 530 nm to around 500 nm, around 520 nm to around 510 nm, around 515 nm or 517 nm and optionally is FAM, and/or wherein one of the first or second fluorophores has an emission wavelength of around 570 nm to around 530 nm, around 560 nm to around 540 nm, around 560 nm or 559 nm, and optionally is HEX.

33. The method of claim 25, wherein the nucleic acid probe comprises a quenching agent, optionally wherein the nucleic acid probe comprises the quenching agent at the 3′ ends, and/or wherein the quenching agent is BHQ1.

34. The method of claim 25, wherein the nucleic acid probe comprises a fluorophore at its 5′ end and a quenching agent at its 3′ end, wherein upon binding of the probe to the amplification product of the PCR assay, the fluorophore is detached from the probe, optionally wherein the nucleic acid probe is a TaqMan probe.

35. The method of claim 25, wherein the amplification product of the rep PCR assay and/or the amplification product of the cap PCR assay is/are detected using a double-stranded DNA intercalating dye, optionally wherein the double-stranded DNA intercalating dye is a fluorescent dye, optionally wherein the double-stranded DNA intercalating dye is an EvaGreen dye.

36. The method of claim 35, wherein the PCR primers capable of hybridising to the rep gene and the primers capable of hybridising to the cap gene have different Tₘ values; and/or wherein the amplification products of the PCR assays to indicate the presence of the rep and the cap gene are of different lengths.

37. The method of claim 25, wherein the rAAV particles are not, or the genomes of the rAAV particles are not, amplified prior to step (a).

38. The method of claim 25, wherein nucleic acid molecules comprised in the rAAV particles are not extracted prior to step (a) and/or (b), and/or wherein the rAAV particles are not disrupted prior to step (a) and/or (b).

39. The method of claim 25, wherein the sample comprising rAAV is obtained from an rcAAV assay, optionally, wherein the sample comprising rAAV is obtained from an rcAAV assay, wherein the method is a method for detecting AAV which may be replication competent pseudo-wild type rAAV particles, wherein the detection of compartments that comprise both a nucleotide sequence corresponding to the at least a portion of the rep gene and a nucleotide sequence corresponding to the at least a portion of the cap gene indicates that the sample comprises rAAV particles which may be pseudo-wild type rcAAV particles, optionally wherein step (d) comprises determining the number of compartments that comprise a nucleotide sequence corresponding to the at least a portion of the rep gene and a nucleotide sequence corresponding to the at least a portion of the cap gene, wherein the number of compartments that comprise both a nucleotide sequence corresponding to the at least a portion of the rep gene and a nucleotide sequence corresponding to the at least a portion of the cap gene indicates the quantity of rAAV in the sample which may be pseudo-wild type rcAAV particles.

40. The method of claim 26, further comprising performing an assay for indicating the presence of a nucleotide sequence corresponding to at least a portion of an inverted terminal repeat (ITR) within each of the plurality of compartments, optionally further comprising detecting any compartments, within the plurality of compartments, which comprise a nucleotide sequence corresponding to at least a portion of an ITR, wherein step (d) comprises determining the number of compartments, within the plurality of compartments, that comprise a nucleotide sequence corresponding to at least a portion of an ITR, optionally wherein step (d) comprises determining the number of compartments that comprise a nucleotide sequence corresponding to at least a portion of an ITR.

41. A kit comprising:
(a) forward and reverse PCR primers capable of hybridising to an AAV rep gene; and
(b) forward and reverse PCR primers capable of hybridising to an AAV cap gene;
optionally wherein the forward and/or reverse PCR primers capable of hybridising to the AAV rep gene are forward and/or reverse PCR primers, and/or wherein the forward and/or reverse PCR primers capable of hybridising to the AAV cap gene are forward and/or reverse PCR primers.

42. The kit of claim 41, further comprising:
(A)
(i) a nucleic acid probe capable of hybridising to a portion of the AAV rep gene for detecting an AAV rep gene; and/or
(ii) a nucleic acid probe capable of hybridising to a portion of the AAV cap gene for detecting an AAV cap gene;
optionally wherein the nucleic acid probe for detecting the AAV rep gene and/or the nucleic acid probe for detecting the AAV cap gene are nucleic acid probes;
or
(B) a double-stranded DNA intercalating dye, optionally wherein the double-stranded DNA intercalating dye is a fluorescent dye, optionally wherein the fluorescent due is an EvaGreen dye.

43. The kit of claim 41, further comprising droplet generator oil; and/or instructions for carrying out: (i) a method for detecting adeno-associated virus (AAV) in a sample comprising recombinant adeno-associated virus (rAAV) particles which may be pseudo-wild type rAAV particles; and (ii) a method of quantifying adeno-associated virus (AAV) in a sample comprising recombinant adeno-associated virus (rAAV) particles which may be pseudo-wild type rAAV particles.


SEQUENCE LISTING (17 sequences)

SEQ ID NO: 1 - DNA, 4679 bp, synthetic construct
  source 1..4679: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 19 bp, synthetic construct
  source 1..19: mol_type = other DNA
cgactttctgacggaatgg

SEQ ID NO: 3 - DNA, 19 bp, synthetic construct
  source 1..19: mol_type = other DNA
cacgtgcatgtggaagtag

SEQ ID NO: 4 - DNA, 20 bp, synthetic construct
  source 1..20: mol_type = other DNA
  misc_feature 1: note = FAM
  misc_feature 20: note = BHQ1
tccggggccttactcacacg

SEQ ID NO: 5 - DNA, 20 bp, synthetic construct
  source 1..20: mol_type = other DNA
gtttacggactcggagtatc

SEQ ID NO: 6 - DNA, 20 bp, synthetic construct
  source 1..20: mol_type = other DNA
tactgagggaccatgaagac

SEQ ID NO: 7 - DNA, 19 bp, synthetic construct
  source 1..19: mol_type = other DNA
  misc_feature 1: note = HEX
  misc_feature 19: note = BHQ1
tggaaacggcgggagacag

SEQ ID NO: 8 - DNA, 20 bp, synthetic construct
  source 1..20: mol_type = other DNA
tatgagcctgactaaaaccg

SEQ ID NO: 9 - DNA, 20 bp, synthetic construct
  source 1..20: mol_type = other DNA
gatcgtacccgtttagttcc

SEQ ID NO: 10 - DNA, 22 bp, synthetic construct
  source 1..22: mol_type = other DNA
  misc_feature 1: note = FAM
  misc_feature 22: note = BHQ1
tggaggacatttccagcaatcg

SEQ ID NO: 11 - DNA, 2211 bp, synthetic construct
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccccattggcacccgttaccttacccgtaacctgtaa

SEQ ID NO: 12 - DNA, 21 bp, synthetic construct
  source 1..21: mol_type = other DNA
tcttcacggactcagactatc

SEQ ID NO: 13 - DNA, 21 bp, synthetic construct
  source 1..21: mol_type = other DNA
tactgaggaatcatgaaaacg

SEQ ID NO: 14 - DNA, 18 bp, synthetic construct
  source 1..18: mol_type = other DNA
  misc_feature 1: note = HEX
  misc_feature 18: note = BHQ1
cagccctcgtgagccgac

SEQ ID NO: 15 - DNA, 19 bp, synthetic construct
  source 1..19: mol_type = other DNA
tggaaacggcgggagacag

SEQ ID NO: 16 - DNA, 22 bp, synthetic construct
  source 1..22: mol_type = other DNA
tggaggacatttccagcaatcg

SEQ ID NO: 17 - DNA, 20 bp, synthetic construct
  source 1..20: mol_type = other DNA
tccggggccttactcacacg